(19) United States
(12) Patent Application Publication
(10) Pub. No.: US 2026/0327684 A1
(43) Pub. Date: Oct. 22, 2026

(54) METHODS FOR NUCLEIC ACID LABELING

(71) Applicant: The General Hospital Corporation, Boston, MA (US)

(72) Inventors: Benjamin Kleinstiver, Boston, MA (US); Connor J. Tou, Boston, MA (US); Benno Orr, Somerville, MA (US)

(21) Appl. No.: 19/474,100

(22) PCT Filed: Apr. 11, 2024
(86) PCT No.: PCT/US2024/024078

§ 371 Date: Oct. 9, 2025

Related U.S. Application Data
(60) Provisional application No. 63/458,519, filed on Apr. 11, 2023.
(60) Provisional application No. 63/590,283, filed on Oct. 13, 2023.

(51) Int. Cl. C12Q 1/6806; C12P 19/34; C12Q 1/6869

(52) U.S. Cl. CPC C12Q 1/6806; C12P 19/34; C12Q 1/6869

(57) ABSTRACT

Described are methods for polymerizing nucleic acid overhangs onto target DNA or RNA molecules via DNA-dependent or RNA-dependent polymerization.

### CLAIM OF PRIORITY

This application is the U.S. National Phase Application under 35 U.S.C. § 371 of International Patent Application No. PCT/US2024/024078, filed on Apr. 11, 2024, which claims the benefit of U.S. Provisional Patent Application Serial Nos. 63/458,519, filed on Apr. 11, 2023, and 63/590,283, filed on Oct. 13, 2023. The entire contents of the foregoing are hereby incorporated by reference.

### FEDERALLY SPONSORED RESEARCH OR DEVELOPMENT

This invention was made with Government support under Grant Nos. HL142494 and CA281401 awarded by the National Institutes of Health. The Government has certain rights in the invention.

### SEQUENCE LISTING

This application contains a Sequence Listing that has been submitted electronically as an XML file named “29539-0737WO1 SL.xml”. The XML file, created on May 27, 2024, is 105,327 bytes in size. The material in the XML file is hereby incorporated by reference in its entirety.

### TECHNICAL FIELD

Described are methods for polymerizing nucleic acid overhangs onto target DNA or RNA molecules via DNA-dependent or RNA-dependent polymerization.

### BACKGROUND

The continued development of nucleic acid sequencing technologies has advanced various fields including genetics, functional genomics, genome editing, transcriptomics, and a range of other basic and medical science disciplines. Despite a dramatic reduction in the per-base cost of sequencing, some individual processes related to the use of these technologies remain inefficient and expensive. For instance, to prepare libraries of nucleic acid samples for sequencing, many approaches require the selective enrichment of target regions of interest via PCR-based amplification (to label the molecules of interest to interact with the sequencing platform and reduce background sequencing; FIG. 1A)¹⁻⁴, or without enrichment they rely on ultra-deep whole-genome or -transcriptome sequencing of the entire sample to achieve sufficient coverage of the region of interest (FIG. 1B). Both methods have caveats, as they can obscure important aspects of the sample (due to PCR bias, PCR error, and the elimination of native base modifications) or add orders-of-magnitude greater cost (due to sequencing the entirety of the sample), respectively.

### SUMMARY

Methods that enable amplification-free enrichment of target DNA sequences in vitro would be transformative for DNA sequencing applications. Although PCR-based amplification of specific molecules can lead to high levels of enrichment, this process creates bias in the eventual sequencing library, eliminates native base modifications, can be prone to intra-molecular template switching and read decomposition, that together result in sequencing data that does not accurately reflect the molecules of interest. Many amplification-free sequencing approaches have sought to solve this challenge, but they suffer from inadequate enrichment for the target molecules, leading to high levels of background (95%+) that reduce sequencing efficiency and throughput. Innovations that improve nucleic acid enrichment would streamline sequencing applications and dramatically reduce cost by avoiding sequencing unwanted off-target nucleic acid molecules. Here we describe methods for nucleic acid labeling or enrichment without amplification by leveraging RNA-programmable nucleases and polymerases. The principles of this approach are (1) user-specifiable sequence-specific nucleic acid cleavage, (2) direct writing of customizable adaptor-complementary ‘overhang’ sequences onto the target substrate nucleic acids of interest to create a modified nucleic acid, and (3) ligation of custom sequencing adaptors onto the nascent custom overhang to create a product nucleic acid (FIG. 1d). The Click-Assisted Genome Enrichment (CAGE) method is applicable to DNA substrates (FIGS. 10A and 10B), whereas the Click-Assisted Precise Targeting of Unaltered RNA for Enrichment (CAPTURE) method is applicable to RNA substrates (FIGS. 10C and 10D). We demonstrate the efficacy of these approaches to write and ligate DNA adaptor-complementary overhangs onto targeted substrate nucleic acids in various one- or two-step molecular reactions with minimal user intervention. This approach is amenable to various modifications and extensible to other nucleic acid modalities or templates (e.g., RNA). The selective polymerization of the overhangs onto only the intended targeted substrate nucleic acids should lead to near perfect enrichment of the desired nucleic acid sequences, substantially reducing background sequencing of unwanted off-target molecules that are highly problematic for current amplification-free approaches. When combined with current short and long-read sequencing platforms, the amplification-free CAGE and CAPTURE methods facilitate a variety of applications including more accurate understanding of DNA and RNA editing outcomes, and more scalable and unbiased interrogation of genomic, epigenomic, transcriptomic, and epitranscriptomic biology.

The present methods use a nuclease with a DNA or RNA template (e.g., either click editor or prime editor) and a DNA- or RNA-dependent polymerase to install a DNA or RNA 3′ overhang onto a target DNA or RNA substrate.

Provided herein are methods comprising: preparing a reaction mixture comprising: (i) a DNA binding domain nuclease (DBDn), optionally Cas9, optionally linked or fused to a clkDNA tethering domain; (ii) one, two, or more guide RNAs (gRNAs) that bind to the DBDn and comprise a spacer comprising a sequence complementary to a selected target sequence on a substrate nucleic acid; (iii) one, two, or more clkDNA oligonucleotide templates (ssDNA, hybrid ssDNA/ssRNA, DNA, hybrid DNA/RNA, or modified DNA or RNA) comprising a localization moiety that binds to the clkDNA tethering domain, a sequence complementary to an adaptor-complementary overhang (ACO) sequence (cACO), and a flap binding region (FBR), and optionally (iv) a DNA-dependent DNA polymerase, optionally wherein the polymerase is linked or fused to the DBDn in the reaction mixture; wherein (i)-(iii) or (i)-(iv) are added to the mixture in any order, and

incubating the reaction mixture under conditions for the gRNAs to bind to the DBDn and the clkDNA oligonucleotide templates to bind to the clkDNA tethering domain on the DBDn.

Additionally, provided herein are methods for generating a modified nucleic acid of DNA comprising 3′ overhangs (e.g., 1-100 bp overhangs) comprising an adaptor-complementary overhang (ACO) sequence. The methods comprise: (A) providing a reaction mixture prepared by a method described herein, comprising: (i) a DNA binding domain nuclease (DBDn), optionally Cas9, optionally linked or fused to a clkDNA tethering domain; (ii) one, two, or more guide RNAs (gRNAs) that bind to the DBDn and comprise a spacer comprising a sequence complementary to a selected target sequence on a substrate nucleic acid; and (iii) one, two, or more clkDNA oligonucleotide templates (ssDNA, hybrid ssDNA/ssRNA, DNA, hybrid DNA/RNA, modified DNA, or modified DNA/RNA bases) comprising a localization moiety that binds to the clkDNA tethering domain, a template complementary to the adaptor-complementary overhang (ACO) sequence (cACO), and a flap binding region (FBR), wherein (i)-(iii) are added to the mixture in any order, and incubating the reaction mixture under conditions for the gRNAs to bind to the DBDn and the clkDNA oligonucleotide templates to bind to the clkDNA tethering domain on the DBDn; (B) providing a sample comprising a substrate nucleic acid DNA, preferably genomic DNA isolated from a cell; and contacting the sample with the reaction mixture of (A) and a DNA-dependent DNA polymerase if the polymerase is absent from the reaction mixture, and incubating the sample under conditions wherein: the nuclease induces a pair of double-stranded breaks with 3′ single stranded flaps; the clkDNA oligonucleotide templates bind to the 3′ single stranded flaps; and the polymerase extends the 3′ single stranded flaps using the ACO complementary region as a template, thereby producing a modified nucleic acid of DNA comprising 3′ overhangs comprising an adaptor-complementary overhang (ACO) sequence. In some embodiments, the substrate nucleic acid are at least 20, 25, 30, or 50 bp, and up to 1, 2, 3, 4, 5, or 6 Mbp.

In some embodiments, the methods for generating a product nucleic acid of DNA having ends comprised of defined adaptor or overhang sequences comprise: providing a sample comprising a modified nucleic acid of DNA comprising 3′ overhangs (e.g., 1-100 bp overhangs) comprising an ACO sequence produced by a method described herein; optionally treating the sample to inactivate any active enzymes, e.g., by heating the sample to above 60° C., e.g., to about 72° C.; contacting the modified nucleic acid of DNA with a partially double stranded DNA oligonucleotide comprising defined adaptor sequences and a 3′ overhang comprising a sequence complementary to the ACO sequence (dsDNA cACO oligo) and a ligase, under conditions sufficient for the ligase to ligate the dsDNA oligo to the modified nucleic acid of DNA (e.g., 20-27° C. or room temperature), thereby producing a product nucleic acid of DNA having ends comprised of defined adaptor sequences.

In some embodiments, the methods for generating a product nucleic acid of DNA having ends comprised of defined adaptor sequences comprise: providing a modified nucleic acid of DNA comprising 3′ overhangs at comprising ACO sequence produced by a method described herein; contacting the modified nucleic acid of DNA with a ligase and a single stranded oligonucleotide modified with a click-compatible moiety at the 5′ end (ssDNA click oligo, hybrid ssDNA/ssRNA, DNA, hybrid DNA/RNA, modified DNA, or modified DNA/RNA bases) comprising a sequence complementary to the ACO sequence under conditions sufficient for the ssDNA click oligo to anneal to the 3′ overhangs and be ligated to the modified nucleic acid of DNA, thereby producing a further modified nucleic acid of DNA having 5′ ends comprising click-compatible moieties; and contacting the further modified nucleic acid of DNA having 5′ ends comprising click-compatible moieties with dsDNA oligos comprising defined adaptor sequences and a 3′ click compatible moiety that reacts with the click-compatible moieties on the further modified nucleic acid of DNA (dsDNA click oligo) to attach the dsDNA click oligos to the ends of the further modified nucleic acid of DNA, thereby producing a product nucleic acid of DNA having ends comprised of defined adaptor sequences.

Also provided herein are methods for generating a product nucleic acid of DNA having ends comprised of defined adaptor sequences comprise: (A) providing a reaction mixture prepared by a method described herein, comprising: (i) a DNA binding domain nuclease (DBDn), optionally Cas9, optionally linked or fused to a clkDNA tethering domain; (ii) one, two, or more guide RNAs (gRNAs) that bind to the DBDn and comprise a spacer comprising a sequence complementary to a selected target sequence; and (iii) one, two, or more clkDNA oligonucleotide templates (i.e., single stranded templates) comprising a localization moiety that binds to the clkDNA tethering domain, a template encoding the adaptor-complementary overhang (ACO) sequence, and a flap binding region (FBR), wherein (i)-(iii) are added to the mixture in any order, and incubating the reaction mixture under conditions for the gRNAs to bind to the DBDn and the clkDNA oligonucleotide templates to bind to the clkDNA tethering domain on the DBDn; (B) providing a sample comprising a substrate nucleic acid DNA, preferably genomic DNA isolated from a cell; and contacting the sample with: the reaction mixture of (A), a DNA-dependent DNA polymerase if the polymerase is absent from the reaction mixture, a double stranded DNA oligonucleotide comprising defined adaptor sequences and a 3′ overhang comprising a sequence complementary to the ACO sequence (dsDNA cACO oligo), and a ligase, optionally a thermostable ligase, incubating the sample under conditions (e.g., 37° C.) wherein: the nuclease induces a pair of double-stranded breaks with 3′ single stranded flaps; the clkDNA oligonucleotide templates bind to the 3′ single stranded flaps; the polymerase extends the 3′ single stranded flaps using the ACO as a template, thereby producing a modified nucleic acid of DNA comprising 3′ overhangs comprising an adaptor-complementary overhang (ACO) sequence, and incubating the sample under conditions wherein the thermostable ligase ligates the dsDNA oligo to the modified nucleic acid of DNA (e.g., 65° C. when a thermostable ligase is used), thereby producing a product nucleic acid of DNA having ends comprised of defined adaptor sequences. In some embodiments, the substrate nucleic acid of DNA is at least 20, 25, 30, or 50 bp, and up to 1, 2, 3, 4, 5, or 6 Mbp.

Also provided herein are methods for generating a product nucleic acid of DNA having ends comprised of defined adaptor sequences. The methods comprise: (A) providing a reaction mixture prepared by a method described herein, comprising: (i) a DNA binding domain nuclease (DBDn), optionally cas9, optionally linked or fused to a clkDNA tethering domain; (ii) one, two, or more guide RNAs (gRNAs) that bind to the DBDn and comprise a spacer comprising a sequence complementary to a selected target sequence; and (iii) one, two, or more clkDNA oligonucleotide templates comprising a localization moiety that binds to the clkDNA tethering domain, a template for the adaptor-complementary overhang (ACO) sequence, and a flap binding region (FBR),

wherein (i)-(iii) are added to the mixture in any order, and incubating the reaction mixture under conditions for the gRNAs to bind to the DBDn and the clkDNA oligonucleotide templates to bind to the clkDNA tethering domain on the DBDn; (B) providing a sample comprising a substrate nucleic acid DNA, preferably genomic DNA isolated from a cell; and contacting the sample with: the reaction mixture of (A), a DNA-dependent DNA polymerase if the polymerase is absent from the reaction mixture, a single stranded DNA oligonucleotide modified with a click-compatible moiety at the 5′ end (ssDNA click oligo) comprising a sequence complementary to the ACO sequence, and a thermostable ligase, incubating the sample under conditions (e.g., 37° C.) wherein: the nuclease induces a pair of double-stranded breaks with 3′ single stranded flaps; the clkDNA oligonucleotide templates bind to the 3′ single stranded flaps; the polymerase extends the 3′ single stranded flaps using the ACO complementary region as a template, thereby producing a modified nucleic acid of DNA comprising 3′ overhangs comprising an adaptor-complementary overhang (ACO) sequence, and incubating the sample under conditions wherein the thermostable ligase is active (e.g., 65° C. for a thermostable ligase, 20-27° C. or room temperature for a non-thermostable ligase), for the ssDNA click oligo to anneal to the 3′ overhangs and be ligated to the modified nucleic acid of DNA, thereby producing a further modified nucleic acid of DNA having 5′ ends comprising click-compatible moieties; and contacting the further modified nucleic acid of DNA having 5′ ends comprising click-compatible moieties with dsDNA oligos comprising defined adaptor sequences and a 3′ click compatible moiety that reacts with the click-compatible moieties on the further modified nucleic acid of DNA (dsDNA click oligo) to attach the dsDNA click oligos to the ends of the further modified nucleic acid of DNA, thereby producing a product nucleic acid of DNA having ends comprised of defined adaptor sequences. In some embodiments, the substrate nucleic acid of DNA is at least 20, 25, 30, or 50 bp, and up to 1, 2, 3, 4, 5, or 6 Mbp.

Additionally, provided herein are methods comprising: preparing a reaction mixture comprising: (i) a DNA binding domain nuclease (DBDn), optionally Cas9; (ii) one, two, or more pegRNAs that bind to the DBDn and comprise a 3′ extension including a primer binding site (PBS) and a reverse transcriptase template (RTT) encoding the complement of the ACO, a scaffold and spacer comprising a sequence complementary to a selected targeted nucleic acid substrate; and optionally (iii) an RNA-dependent polymerase, optionally wherein the polymerase is linked or fused to the DBDn in the reaction mixture; wherein (i)-(ii) or (i)-(iii) are added to the mixture in any order, and incubating the reaction mixture under conditions for the gRNAs to bind to the DBDn.

Additionally, provided herein are methods for generating a modified nucleic acid of DNA comprising 3′ overhangs (e.g., 1-100 bp overhangs) comprising an adaptor-complementary overhang (ACO) sequence. The methods comprise (A) providing a reaction mixture prepared according to a method described herein, comprising (i) a DNA binding domain nuclease (DBDn), optionally Cas9; (ii) one, two, or more pegRNAs that bind to the DBDn and comprise a 3′ extension including a primer binding site (PBS) and a reverse transcriptase template (RTT) encoding the complement of the ACO, a scaffold and spacer comprising a sequence complementary to a selected target sequence; and optionally (iii) an RNA-dependent polymerase, optionally wherein the polymerase is linked or fused to the DBDn in the reaction mixture; wherein (i)-(ii) or (i)-(iii) are added to the mixture in any order, and incubating the reaction mixture under conditions for the gRNAs to bind to the DBDn; (B) providing a sample comprising a substrate nucleic acid DNA, preferably genomic DNA isolated from a cell; and contacting the sample with the reaction mixture of (A) and an RNA-dependent DNA polymerase if the polymerase is absent from the reaction mixture, incubating the sample under conditions wherein: the nuclease induces a pair of double-stranded breaks with 3′ single stranded flaps; the clkDNA oligonucleotide templates bind to the 3′ single stranded flaps; and the polymerase extends the 3′ single stranded flaps using the ACO complementary region as a template, thereby producing a modified nucleic acid of DNA comprising 3′ overhangs comprising an adaptor-complementary overhang (ACO) sequence. In some embodiments, the substrate nucleic acid of DNA is at least 20, 25, 30, or 50 bp, and up to 1, 2, 3, 4, 5, or 6 Mbp.

Further, provided herein are methods of generating a product nucleic acid of DNA having ends comprised of defined adaptor sequences. The methods comprise: providing a sample comprising a modified nucleic acid of DNA comprising 3′ overhangs (e.g., 1-100 bp overhangs) comprising an ACO sequence produced by a method described herein; optionally treating the sample to inactivate any active enzymes, e.g., by heating the sample to about 72° C.; contacting the modified nucleic acid of DNA with a partially double stranded DNA oligonucleotide comprising defined adaptor sequences and a 3′ overhang comprising a sequence complementary to the ACO sequence (dsDNA cACO oligo) and a ligase, under conditions sufficient for the ligase to ligate the dsDNA oligo to the modified nucleic acid of DNA (e.g., 20-27° C. or room temperature), thereby producing a product nucleic acid of DNA having ends comprised of defined adaptor sequences. In some embodiments, the substrate nucleic acid of DNA is at least 20, 25, 30, or 50 bp, and up to 1, 2, 3, 4, 5, or 6 Mbp.

Also provided herein are methods of generating a product nucleic acid of DNA having ends comprised of defined adaptor sequences. The methods comprise: providing a modified nucleic acid of DNA comprising 3′ overhangs (e.g., 1-100 bp overhangs) comprising an ACO sequence produced by a method described herein; contacting the modified nucleic acid of DNA with a ligase and a single stranded DNA oligonucleotide modified with a click-compatible moiety at the 5′ end (ssDNA click oligo) comprising a sequence complementary to the ACO sequence under conditions sufficient for the ssDNA click oligo to anneal to the 3′ overhangs and be ligated to the modified nucleic acid of DNA, thereby producing a further modified nucleic acid of DNA having 5′ ends comprising click-compatible moieties; and contacting the further modified nucleic acid of DNA having 5′ ends comprising click-compatible moieties with dsDNA oligos comprising defined adaptor sequences and a 3′ click compatible moiety that reacts with the click-compatible moieties on the further modified nucleic acid (dsDNA click oligo) to attach the dsDNA click oligos to the ends of the further modified nucleic acid, thereby producing a product nucleic acid of DNA having ends comprised of defined adaptor sequences. In some embodiments, the substrate nucleic acid of DNA is at least 20, 25, 30, or 50 bp, and up to 1, 2, 3, 4, 5, or 6 Mbp.

Additionally provided herein are methods for generating a product nucleic acid of DNA having ends comprised of defined adaptor sequences. The methods comprise: (A) providing a reaction mixture prepared by a method described herein, comprising (i) a DNA binding domain nuclease (DBDn), optionally Cas9; (ii) one, two, or more pegRNAs that bind to the DBDn and comprise a 3′ extension including a primer binding site (PBS) and a reverse transcriptase template (RTT) encoding the complement of the ACO, a scaffold and spacer comprising a sequence complementary to a selected target sequence; and optionally (iii) an RNA-dependent polymerase, optionally wherein the polymerase is linked or fused to the DBDn in the reaction mixture; wherein (i)-(ii) or (i)-(iii) are added to the mixture in any order, and incubating the reaction mixture under conditions for the gRNAs to bind to the DBDn; (B) providing a sample comprising a substrate nucleic acid DNA, preferably genomic DNA isolated from a cell; and contacting the sample with: the reaction mixture of (A), a RNA-dependent DNA polymerase if the polymerase is absent from the reaction mixture, a double stranded DNA oligonucleotide comprising defined adaptor sequences and a 3′ overhang (e.g., 1-100 bp overhangs) comprising a sequence complementary to the ACO sequence (dsDNA cACO oligo), and a ligase, optionally a thermostable ligase, incubating the sample under conditions (e.g., 37° C.) wherein: the nuclease induces a pair of double-stranded breaks with 3′ single stranded flaps; the clkDNA oligonucleotide templates bind to the 3′ single stranded flaps; the polymerase extends the 3′ single stranded flaps using the ACO complementary region as a template, thereby producing a modified nucleic acid of DNA comprising 3′ overhangs (e.g., 1-100 bp overhangs) comprising an adaptor-complementary overhang (ACO) sequence, and incubating the sample under conditions (e.g., 65° C. for a thermostable ligase, 20-27° C. or room temperature for non-thermostable ligase) wherein the thermostable ligase ligates the dsDNA oligo to the nucleic acid of DNA, thereby producing a product nucleic acid of DNA having ends comprised of defined adaptor sequences. In some embodiments, the substrate nucleic acid of DNA is at least 20, 25, 30, or 50 bp, and up to 1, 2, 3, 4, 5, or 6 Mbp.

Also provided herein are methods for generating a product nucleic acid of DNA having ends comprised of defined adaptor sequences. The methods comprise: (A) providing a reaction mixture prepared by a method described herein, comprising (i) a DNA binding domain nuclease (DBDn), optionally Cas9; (ii) one, two, or more pegRNAs that bind to the DBDn and comprise a 3′ extension including a primer binding site (PBS) a reverse transcriptase template (RTT) encoding the ACO, a scaffold and spacer comprising a sequence complementary to a selected target sequence; and optionally (iii) an RNA-dependent polymerase, optionally wherein the polymerase is linked or fused to the DBDn in the reaction mixture; wherein (i)-(ii) or (i)-(iii) are added to the mixture in any order, and incubating the reaction mixture under conditions for the gRNAs to bind to the DBDn; (B) providing a sample comprising a substrate nucleic acid DNA, preferably genomic DNA isolated from a cell; and contacting the sample with: the reaction mixture of (A), a RNA-dependent DNA polymerase if the polymerase is absent from the reaction mixture, a single stranded DNA oligonucleotide modified with a click-compatible moiety at the 5′ end (ssDNA click oligo) comprising a sequence complementary to the ACO sequence, and a thermostable ligase, incubating the sample under conditions (e.g., 37° C.) wherein: the nuclease induces a pair of double-stranded breaks with 3′ single stranded flaps; the clkDNA oligonucleotide templates bind to the 3′ single stranded flaps; the polymerase extends the 3′ single stranded flaps using the ACO complementary region as a template, thereby producing a modified nucleic acid of DNA comprising 3′ overhangs (e.g., 1-100 bp overhangs) comprising an adaptor-complementary overhang (ACO) sequence, and incubating the sample under conditions wherein the thermostable ligase is active (e.g., 65° C.), for the ssDNA click oligo to anneal to the 3′ overhangs and be ligated to the modified nucleic acid of DNA, thereby producing a further modified nucleic acid of DNA having 5′ ends comprising click-compatible moieties; and contacting the further modified nucleic acid of DNA having 5′ ends comprising click-compatible moieties with dsDNA oligos comprising defined adaptor sequences and a 3′ click compatible moiety that reacts with the click-compatible moieties on the further modified nucleic acid of DNA (dsDNA click oligo) to attach the dsDNA click oligos to the ends of the further modified nucleic acid of DNA, thereby producing a product nucleic acid of DNA having ends comprised of defined adaptor sequences. In some embodiments, the substrate nucleic acid of DNA are at least 20, 25, 30, or 50 bp, and up to 1, 2, 3, 4, 5, or 6 Mbp.

Further provided herein are methods comprising: preparing a reaction mixture comprising: (i) a RNA binding domain nuclease (RBDn), optionally Cas13, optionally linked or fused to a clkDNA tethering domain; (ii) at least one guide RNA (gRNA) that binds to the RBDn and comprises a spacer comprising a sequence complementary to a selected target sequence; (iii) at least one clkDNA oligonucleotide template comprising a localization moiety that binds to the clkDNA tethering domain, a template encoding the complement of the adaptor-complementary overhang (ACO) sequence, and a flap binding region (FBR), and optionally (iv) a DNA-dependent DNA polymerase, optionally wherein the polymerase is linked or fused to the RBDn in the reaction mixture; wherein (i)-(iii) or (i)-(iv) are added to the mixture in any order, and incubating the reaction mixture under conditions for the gRNAs to bind to the RBDn and the clkDNA oligonucleotide templates to bind to the clkDNA tethering domain on the RBDn.

Also provided herein are methods for generating a modified nucleic acid of RNA comprising a 3′ end comprising an adaptor-complementary overhang (ACO) sequence, the method comprising: (A) providing a reaction mixture prepared by a method described herein, comprising: (i) a RNA binding domain nuclease (RBDn), optionally Cas13, optionally linked or fused to a clkDNA tethering domain; (ii) at least one guide RNA (gRNA) that binds to the RBDn and comprises a spacer comprising a sequence complementary to a selected target sequence; (iii) at least one clkDNA oligonucleotide template comprising a localization moiety that binds to the clkDNA tethering domain, a template encoding the complement of the adaptor-complementary overhang (ACO) sequence, and a flap binding region (FBR), and optionally (iv) a DNA-dependent DNA polymerase, optionally wherein the polymerase is linked or fused to the RBDn in the reaction mixture; wherein (i)-(iii) or (i)-(iv) are added to the mixture in any order, and incubating the reaction mixture under conditions for the gRNAs to bind to the RBDn and the clkDNA oligonucleotide templates to bind to the clkDNA tethering domain on the RBDn; (B) providing a sample comprising substrate nucleic acid of RNA, preferably RNA isolated from a cell or sample from an animal (e.g., mRNA or purified RNA); and contacting the sample with the reaction mixture of (A) and a DNA-dependent DNA polymerase if the polymerase is absent from the reaction mixture, incubating the sample under conditions wherein: the clkDNA oligonucleotide template binds to the RNA; and the polymerase extends the 3′ end of the RNA molecule using the ACO complementary region of the clkDNA as a template, thereby producing a modified nucleic acid of RNA comprising a 3′ end comprising an adaptor-complementary overhang (ACO) sequence. In some embodiments, the substrate nucleic acid of RNA are at least 20, 25, 30, or 50 bp, and up to 1, 2, 3, 4, 5, or 6 Mbp.

Additionally provided herein are methods for generating a product nucleic acid of DNA having ends comprised of defined adaptor sequences. The method comprise: providing a sample comprising a modified nucleic acid of DNA comprising a 3′ end comprising an ACO sequence produced by a method described herein; optionally treating the sample to inactivate any active enzymes, e.g., by heating the sample to about 72° C.;

contacting the modified nucleic acid with a partially double stranded DNA oligonucleotide comprising defined adaptor sequences and a 3′ overhang (e.g., 1-100 bp overhang) comprising a sequence complementary to the ACO sequence (dsDNA cACO oligo) and a ligase, under conditions sufficient for the ligase to ligate the dsDNA oligo to the modified nucleic acid (e.g., 20-27° C. or room temperature for non-thermostable ligase), thereby producing a further modified nucleic acid of DNA having ends comprised of defined adaptor sequences. In some embodiments, the substrate nucleic acid of DNA is at least 20, 25, 30, or 50 bp, and up to 1, 2, 3, 4, 5, or 6 Mbp.

Also provided herein are methods of generating a product nucleic acid of DNA having ends comprised of defined adaptor sequences. The methods comprise: providing a modified nucleic acid of DNA comprising a 3′ end comprising an ACO sequence produced by a method described herein; contacting the modified nucleic acid with a ligase and a single stranded DNA oligonucleotide modified with a click-compatible moiety at the 5′ end (ssDNA click oligo) comprising a sequence complementary to the ACO sequence under conditions sufficient for the ssDNA click oligo to anneal to the 3′ end and be ligated to the modified nucleic acid, thereby producing a further modified nucleic acid of dsDNA having a 5′ end comprising a click-compatible moiety; and contacting the further modified nucleic acid of DNA having a 5′ end comprising a click-compatible moiety with dsDNA oligos comprising defined adaptor sequences and a 3′ click compatible moiety that reacts with the click-compatible moieties on the further modified nucleic acid of DNA (dsDNA click oligo) to attach the dsDNA click oligos to the ends of the further modified nucleic acid of DNA, thereby producing a product nucleic acid of DNA having ends comprised of defined adaptor sequences. In some embodiments, the substrate nucleic acid of DNA is at least 20, 25, 30, or 50 bp, and up to 1, 2, 3, 4, 5, or 6 Mbp.

Further provided herein are methods for generating a product nucleic acid of RNA having ends comprised of defined adaptor sequences comprising: (A) providing a reaction mixture prepared by a method described herein, comprising: (i) a RNA binding domain nuclease (RBDn), optionally Cas13, optionally linked or fused to a clkDNA tethering domain; (ii) at least one guide RNA (gRNA) that binds to the RBDn and comprises a spacer comprising a sequence complementary to a selected target sequence;

(iii) at least one clkDNA oligonucleotide template comprising a localization moiety that binds to the clkDNA tethering domain, a template encoding the complement of the adaptor-complementary overhang (ACO) sequence, and a flap binding region (FBR), and optionally (iv) a DNA-dependent DNA polymerase, optionally wherein the polymerase is linked or fused to the RBDn in the reaction mixture; wherein (i)-(iii) or (i)-(iv) are added to the mixture in any order, and incubating the reaction mixture under conditions for the gRNAs to bind to the RBDn and the clkDNA oligonucleotide templates to bind to the clkDNA tethering domain on the RBDn; (B) providing a sample comprising RNA; and contacting the sample with: the reaction mixture of (A), a RNA-dependent DNA polymerase if the polymerase is absent from the reaction mixture, a double stranded DNA oligonucleotide comprising defined adaptor sequences and a 3′ overhang (e.g., 1-100 bp overhang) comprising a sequence complementary to the ACO sequence (dsDNA cACO oligo), and a ligase, optionally a thermostable ligase, incubating the sample under conditions (e.g., 37° C.) wherein: the clkDNA oligonucleotide templates bind to the RNA; the polymerase extends the 3′ end using the ACO complementary region as a template, thereby producing a modified nucleic acid of RNA comprising a ds 3′ end comprising an adaptor-complementary overhang (ACO) sequence, and incubating the sample under conditions (e.g., 65° C. for a thermostable ligase, 20-27° C. or room temperature for non-thermostable ligase) wherein the thermostable ligase ligates the dsDNA oligo to the modified nucleic acid of RNA, thereby producing a product nucleic acid of RNA having ends comprised of defined adaptor sequences. In some embodiments, the substrate nucleic acid of RNA is at least 20, 25, 30, or 50 bp, and up to 1, 2, 3, 4, 5, or 6 Mbp.

A method for generating a product nucleic acid (e.g., RNA) having ends comprised of defined adaptor sequences, the method comprising: (A) providing a reaction mixture prepared by a method described herein, comprising: (i) a RNA binding domain nuclease (RBDn), optionally Cas13, optionally linked or fused to a clkDNA tethering domain; (ii) at least one guide RNA (gRNA) that binds to the RBDn and comprises a spacer comprising a sequence complementary to a selected target sequence; (iii) at least one clkDNA oligonucleotide template comprising a localization moiety that binds to the clkDNA tethering domain, a template encoding the complement of the adaptor-complementary overhang (ACO) sequence, and a flap binding region (FBR), and optionally (iv) a DNA-dependent DNA polymerase, optionally wherein the polymerase is linked or fused to the RBDn in the reaction mixture; wherein (i)-(iii) or (i)-(iv) are added to the mixture in any order, and incubating the reaction mixture under conditions for the gRNAs to bind to the RBDn and the clkDNA oligonucleotide templates to bind to the clkDNA tethering domain on the RBDn; (B) providing a sample comprising RNA; and contacting the sample with: the reaction mixture of (A), a RNA-dependent DNA polymerase if the polymerase is absent from the reaction mixture, a single stranded DNA oligonucleotide modified with a click-compatible moiety at the 5′ end (ssDNA click oligo) comprising a sequence complementary to the ACO sequence, and a thermostable ligase, incubating the sample under conditions (e.g., 37° C.) wherein: the clkDNA oligonucleotide templates bind to the RNA; the polymerase extends the 3′ end using the ACO complementary region as a template, thereby producing a modified nucleic acid comprising a 3′ end comprising an adaptor-complementary overhang (ACO) sequence, and incubating the sample under conditions (e.g., 65° C. for a thermostable ligase, 20-27° C. or room temperature for non-thermostable ligase) wherein the thermostable ligase is active, for the ssDNA click oligo to anneal to the 3′ end and be ligated to the modified nucleic acid, thereby producing a region of dsDNA having a 5′ end comprising a click-compatible moiety; and contacting the region of dsDNA having a 5′ end comprising a click-compatible moiety with dsDNA oligos comprising defined adaptor sequences and a 3′ click compatible moiety that reacts with the click-compatible moieties on the further modified nucleic acid (dsDNA click oligo) to attach the dsDNA click oligos to the ends of the further modified nucleic acid, thereby producing a product nucleic acid having ends comprised of defined adaptor sequences. In some embodiments, the substrate nucleic acid is at least 20, 25, 30, or 50 bp, and up to 1, 2, 3, 4, 5, or 6 Mbp.

Further, provided herein are methods comprising: preparing a reaction mixture comprising: (i) a RNA binding domain nuclease (RBDn), optionally Cas13; (ii) at least one pegRNA that binds to the RBDn and comprise a 3′ extension including a primer binding site (PBS) a reverse transcriptase template (RTT) encoding the ACO, a scaffold and spacer comprising a sequence complementary to a selected target sequence; and optionally (iii) an RNA-dependent polymerase, optionally wherein the polymerase is linked or fused to the RBDn in the reaction mixture; wherein (i)-(ii) or (i)-(iii) are added to the mixture in any order, and incubating the reaction mixture under conditions for the gRNAs to bind to the RBDn.

Also provided herein are methods for generating a further modified nucleic acid of RNA comprising a 3′ end comprising an adaptor-complementary overhang (ACO) sequence, the method comprising: (A) providing a reaction mixture prepared according a method described herein, comprising (i) a RNA binding domain nuclease (RBDn), optionally Cas13; (ii) at least one pegRNA that binds to the RBDn and comprise a 3′ extension including a primer binding site (PBS) a reverse transcriptase template (RTT) encoding a sequence complementary to the ACO, a scaffold and spacer comprising a sequence complementary to a selected target sequence; and optionally (iii) an RNA-dependent polymerase, optionally wherein the polymerase is linked or fused to the RBDn in the reaction mixture; wherein (i)-(ii) or (i)-(iii) are added to the mixture in any order, and incubating the reaction mixture under conditions for the gRNAs to bind to the RBDn; (B) providing a sample comprising RNA; and contacting the sample with the reaction mixture of (A) and an RNA-dependent DNA polymerase if the polymerase is absent from the reaction mixture, incubating the sample under conditions wherein: the clkDNA oligonucleotide templates bind to the RNA; and the polymerase extends the 3′ end using the ACO complementary region as a template, thereby producing a modified nucleic acid of RNA comprising a 3′ end comprising an adaptor-complementary overhang (ACO) sequence. In some embodiments, the substrate nucleic acid is at least 20, 25, 30, or 50 bp, and up to 1, 2, 3, 4, 5, or 6 Mbp.

Further, provided herein are methods of generating a product nucleic acid of DNA having ends comprised of defined adaptor sequences comprising: providing a sample comprising a modified nucleic acid of DNA comprising a 3′ end comprising ACO sequence produced by a method described herein; optionally treating the sample to inactivate any active enzymes, e.g., by heating the sample to about 72° C.; contacting the modified nucleic acid of DNA with a double stranded DNA oligonucleotide comprising defined adaptor sequences and a ssDNA 3′ overhang (e.g., 1-100 bp overhang) comprising a sequence complementary to the ACO sequence (dsDNA cACO oligo) and a ligase, under conditions (e.g., 20-27° C. or room temperature for non-thermostable ligase) sufficient for the ligase to ligate the dsDNA oligo to the modified nucleic acid of DNA, thereby producing a region of dsDNA having ends comprised of defined adaptor sequences. In some embodiments, the substrate nucleic acid of DNA is at least 20, 25, 30, or 50 bp, and up to 1, 2, 3, 4, 5, or 6 Mbp.

Additionally provided herein are methods of generating a product nucleic acid of DNA having ends comprised of defined adaptor sequences. The methods comprise: providing a modified nucleic acid of DNA comprising 3′ overhangs (e.g., 1-100 bp overhangs) comprising an ACO sequence produced by a method described herein; contacting the modified nucleic acid with a ligase and a single stranded DNA oligonucleotide modified with a click-compatible moiety at the 5′ end (ssDNA click oligo) comprising a sequence complementary to the ACO sequence under conditions sufficient for the ssDNA click oligo to anneal to the 3′ overhangs and be ligated to the modified nucleic acid, thereby producing a further modified nucleic acid of DNA having 5′ ends comprising click-compatible moieties; and contacting the further modified nucleic acid of DNA having 5′ ends comprising click-compatible moieties with dsDNA oligos comprising defined adaptor sequences and a 3′ click compatible moiety that reacts with the click-compatible moieties on the further modified nucleic acid of DNA (dsDNA click oligo) to attach the dsDNA click oligos to the ends of the further modified nucleic acid, thereby producing a product nucleic acid of DNA having ends comprised of defined adaptor sequences. In some embodiments, the substrate nucleic acid of DNA is at least 20, 25, 30, or 50 bp, and up to 1, 2, 3, 4, 5, or 6 Mbp.

Also provided herein are methods for generating a product nucleic acid of RNA having ends comprised of defined adaptor sequences. The methods comprise: (A) providing a reaction mixture prepared by a method described herein, comprising (i) a RNA binding domain nuclease (RBDn), optionally Cas13; (ii) one, two, or more pegRNAs that bind to the RBDn and comprise a 3′ extension including a primer binding site (PBS) a reverse transcriptase template (RTT) encoding the ACO, a scaffold and spacer comprising a sequence complementary to a selected target sequence; and optionally (iii) an RNA-dependent polymerase, optionally wherein the polymerase is linked or fused to the RBDn in the reaction mixture; wherein (i)-(ii) or (i)-(iii) are added to the mixture in any order, and incubating the reaction mixture under conditions for the gRNAs to bind to the RBDn; (B) providing a sample comprising RNA; and

contacting the sample with: the reaction mixture of (A), a RNA-dependent DNA polymerase if the polymerase is absent from the reaction mixture, a double stranded DNA oligonucleotide comprising defined adaptor sequences and a 3′ overhang (e.g., 1-100 bp overhang) comprising a sequence complementary to the ACO sequence (dsDNA cACO oligo), and a ligase, optionally a thermostable ligase, incubating the sample under conditions (e.g., 37° C.) wherein: the clkDNA oligonucleotide templates bind to the RNA; the polymerase extends the 3′ end using the ACO complementary region as a template, thereby producing a modified nucleic acid of RNA comprising a 3′ end comprising an adaptor-complementary overhang (ACO) sequence, and incubating the sample under conditions (e.g., 65° C. for a thermostable ligase, 20-27° C. or room temperature for non-thermostable ligase) wherein the thermostable ligase ligates the dsDNA oligo to the modified nucleic acid of RNA, thereby producing a modified nucleic acid of RNA having an end comprised of a defined adaptor sequence. In some embodiments, the substrate nucleic acid of RNA is at least 20, 25, 30, or 50 bp, and up to 1, 2, 3, 4, 5, or 6 Mbp.

A method for generating a product nucleic acid, e.g., RNA, having ends comprised of defined adaptor sequences, the method comprising: (A) providing a reaction mixture prepared by a method described herein, comprising (i) a RNA binding domain nuclease (RBDn), optionally Cas13; (ii) one, two, or more pegRNAs that bind to the RBDn and comprise a 3′ extension including a primer binding site (PBS) a reverse transcriptase template (RTT) encoding a sequence complementary to the ACO, a scaffold and spacer comprising a sequence complementary to a selected target sequence; and optionally (iii) an RNA-dependent polymerase, optionally wherein the polymerase is linked or fused to the RBDn in the reaction mixture; wherein (i)-(ii) or (i)-(iii) are added to the mixture in any order, and incubating the reaction mixture under conditions for the gRNAs to bind to the RBDn; (B) providing a sample comprising RNA; and contacting the sample with: the reaction mixture of (A), a RNA-dependent DNA polymerase if the polymerase is absent from the reaction mixture, a single stranded DNA oligonucleotide modified with a click-compatible moiety at the 5′ end (ssDNA click oligo) comprising a sequence complementary to the ACO sequence, and a thermostable ligase, incubating the sample under conditions (e.g., 37° C.) wherein: the clkDNA oligonucleotide templates bind to the RNA; the polymerase extends the 3′ single stranded flaps using the ACO complementary region as a template, thereby producing a modified nucleic acid, comprising a 3′ end comprising an adaptor-complementary overhang (ACO) sequence, and incubating the sample under conditions (e.g., 65° C. for a thermostable ligase, 20-27° C. or room temperature for non-thermostable ligase) wherein the thermostable ligase is active, for the ssDNA click oligo to anneal to the 3′ end and be ligated to the modified nucleic acid, thereby producing a region of dsDNA having 5′ ends comprising click-compatible moieties; and contacting the further modified nucleic acid having 5′ ends comprising click-compatible moieties with dsDNA oligos comprising defined adaptor sequences and a 3′ click compatible moiety that reacts with the click-compatible moieties on the further modified nucleic acid (dsDNA click oligo) to attach the dsDNA click oligos to the ends of the further modified nucleic acid of RNA, thereby producing a further modified nucleic acid having ends comprised of defined adaptor sequences. In some embodiments, the substrate nucleic acid is least 20, 25, 30, or 50 bp, and up to 1, 2, 3, 4, 5, or 6 Mbp.

Additionally provided are methods for labeling DNA or RNA as described herein.

Also provided herein are methods comprising: cleaving a nucleic acid using a sequence-specific nuclease (e.g., via a nuclease-active PCE construct comprising a CRISPR-Cas nuclease directed by a gRNA); writing adaptor-complementary overhang (ACO) sequences onto a 3′ end of a cleaved nucleic acids (e.g., in a DNA version of this method via a DNA-dependent DNA polymerase of the PCE using the clkDNA as a template); and ligating an oligonucleotide, e.g., a sequencing adaptor, onto the 3′ ACO.

In some embodiments, the nucleic acid is DNA, and the nuclease is a Class II type II CRISPR, optionally CRISPR-Cas9.

In some embodiments, the nucleic acid is RNA, and the nuclease is a class II type VI CRISPR, optionally CRISPR-Cas13.

In some embodiments, the method further comprises sequencing the nucleic acid using the sequencing adaptor.

In some of the methods described herein, the DBDn can be a Cas-family enzymes (e.g., Cas9 or Cas12), a TnpB-family enzyme, or an IscB-family enzyme, e.g., as described herein.

As used herein, “linked” can include non-covalent attachment or association such as recruitment of the clkDNA through the gRNA (e.g., via RNA hairpin on the gRNA and RNA aptamer binding protein fused to a HUH, enabling recruitment of the clkDNA to Cas9 via the gRNA).

In some embodiments of the methods described herein, a plurality of different ACOs are used; in such methods, preferably a plurality of adaptors are used that correspond or are fully or partly complementary in sequence to the different ACOs (e.g., if two different ACO sequences are used, two different adaptor sequences are present in the mix, each complementary or partly complementary (e.g., at least 50%, 60%, 70%, 80%, 90%, or 95%, complementary) to one of the ACO sequences).

In some embodiments of the methods described herein, the gRNAs are allowed to bind to the DBDn before the clkDNA oligonucleotide templates are added to the mixture.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A-D. Overview of methods for target molecule enrichment and sequencing. A, Schematic of amplification-based methods to enrich for target regions or to add sequencer-specific adapter sequences. B, Schematic of ultra-deep sequencing to permit adequate coverage of a region of interest. C, Schematic of current Cas9-targeted, phosphorylation-based enrichment approach. D, Schematic of one embodiment of a CAGE sequence-specific enrichment approach, leading to more selective background-free target substrate enrichment without amplification via user-specifiable adapter-complementary overhangs (ACOs). ROI, region of interest; DNA, deoxyribonucleic acid; RNA, ribonucleic acid

FIG. 2A-C. Workflow for exemplary CAGE and sequence-specific adaptor ligation to DNA targets. A, Ribonucleoprotein (RNP) formation of exemplary Click Editors using a DNA-binding domain nuclease (DBDn), HUH endonuclease, and DNA-dependent polymerase complex (where the DNA polymerase can be unfused or fused to the DBDn) in combination with a guide RNA (gRNA) and clkDNA pair. B, CAGE workflow using adaptors that contain a 3′ overhang complementary to the adaptor-complementary overhang (ACO) region of the modified DNA. C, CAGE workflow using oligo annealing to the ACO and subsequent adaptor attachment via click chemistry. ACO, adaptor-complementary overhang; cACO, complement of the ACO; gRNA, guide RNA; clkDNA, click DNA template oligonucleotide; FBR, flap-binding region; PT, polymerization template; RNP, ribonucleoprotein; DBDn, DNA binding domain nuclease; DNA, deoxyribonucleic acid; HUH, a histidine-hydrophobic residue-histidine (HUH) motif-containing endonuclease.

FIG. 3A-B. Detailed exemplary 6-step and 5-step CAGE workflow. A, Schematic of an exemplary 6-step CAGE workflow. A heat inactivation step (step 4) dissociates Cas9 from the target DNA, liberating the click-edited ACO for adaptor ligation in subsequent steps. The DNA Ligase and adaptors are subsequently added in step 5. B, Schematic of an exemplary 5-step CAGE workflow. DNA ligase and adaptor are added together with other components in a single reaction prior to click editing. No heat inactivation step is included. ACO, adaptor-complementary overhang; cACO, complement of the ACO; sgRNA, single guide RNA; gRNA, guide RNA; clkDNA, click DNA template oligonucleotide; FBR, flap-binding region; PT, polymerization template; DBDn, DNA binding domain nuclease; RNP, ribonucleoprotein; DNA, deoxyribonucleic acid; HUH, a histidine-hydrophobic residue-histidine (HUH) motif-containing endonuclease; RT, room temperature; dNTPs, deoxynucleoside triphosphates; ATP, adenosine triphosphate.

FIG. 4A-G (SEQ ID NO: 94-95). CAGE-mediated adaptor ligation on PCR-generated fragments and human genomic DNA. A, Schematic of exemplary single-end CAGE workflow to assess feasibility of sequence-specific ACO addition to a substrate followed by adaptor ligation. B, Junction PCR across the adaptor:target junction of the product DNA to verify adaptor ligation following CAGE reactions on PCR-fragments. Bands illustrating PCR amplification are only present in conditions that have the adaptor successfully ligated; assessed via Qiaxcel capillary electrophoresis (Qiagen). C, Sanger sequencing results of the expected product (from a 6-step CAGE reaction) from PCR substate targeting experiments. D, Adaptor ligation efficiency assessed by ddPCR for 5- and 6-step reactions (against a PCR substrate) and reactions that varied the click editing incubation time. Individual datapoints, mean, and standard deviation shown for n=3 replicates. E, Junction PCR across the adaptor:target junction of the product DNA for human gDNA targeting (HEK site 3) experiments. Bands illustrating PCR amplification are only present in conditions that have the adaptor successfully ligated; assessed via Qiaxcel capillary electrophoresis (Qiagen). F, Sanger sequencing results of the expected product (from 6-step CAGE reactions) from gDNA targeting experiments. G, Adaptor ligation efficiency from gDNA targeting experiments assessed by ddPCR for 6-step CAGE reactions using a 2 hour click editing time. Individual datapoints, mean, and standard deviation shown for n=3 replicates. ACO, adaptor-complementary overhang; cACO, complement of the ACO; gRNA, guide RNA; clkDNA, click DNA template oligonucleotide; FBR, flap-binding region; PT, polymerization template; RNP, ribonucleoprotein; DNA, deoxyribonucleic acid; HUH, a histidine-hydrophobic residue-histidine (HUH) motif-containing endonuclease; RT, room temperature; dNTPs, deoxynucleoside triphosphates; ATP, adenosine triphosphate; PCR, polymerase chain reaction; CAGE, click-assisted genome enrichment.

FIG. 5. Use of thermostable DNA ligases in CAGE reactions. Exemplary 5-step CAGE workflow, similar to as shown in FIG. 3B, except that thermostable DNA ligases used; thermostable ligases are activated at the same temperature that the DBDn is inactivated, leading to single-pot click editing and adaptor ligation without user intervention. ACO, adaptor-complementary overhang; cACO, complement of the ACO; gRNA, guide RNA; sgRNA, single guide RNA; clkDNA, click DNA template oligonucleotide; FBR, flap-binding region; PT, polymerization template; DBDn, DNA binding domain nuclease; RNP, ribonucleoprotein; DNA, deoxyribonucleic acid; HUH, a histidine-hydrophobic residue-histidine (HUH) motif-containing endonuclease; RT, room temperature; dNTPs, deoxynucleoside triphosphates; ATP, adenosine triphosphate.

FIG. 6A-C. Workflow for PE-CAGE and sequence-specific adaptor ligation to DNA targets. A, Ribonucleoprotein (RNP) formation of Prime Editors using a DNA-binding domain nuclease (DBDn) and RNA-dependent polymerase (e.g. reverse transcriptase (RT); where the RT can be unfused or fused to the DBDn) in combination with a prime editing guide RNA (pegRNA) and clkDNA pair. B, PE-CAGE workflow using adaptors that contain a 3′ overhang complementary to the adaptor-complementary overhang (ACO) region of the target DNA. C, PE-CAGE workflow using oligo annealing to the ACO and subsequent adaptor attachment via click chemistry. cACO, complement of the ACO; PBS, primer binding site; RTT, reverse transcriptase template.

FIG. 7A-C(SEQ ID NO: 96). PE-CAGE-mediated overhang installation on human genomic DNA. A, Schematic of a single-end PE-CAGE experiment to assess feasibility of ACO installation using a human genomic DNA substrate (at HEK site 3). B, Junction PCR (across the ACO:gDNA target junction) and Sanger sequencing results of the modified nucleic acid showing the expected ACO installation at the target site. C, ACO installation efficiency on genomic DNA at HEK site 3, as assessed by ddPCR. ACO, adaptor-complementary overhang; cACO, complement of the ACO; RT, reverse transcriptase; PBS, primer binding site; RTT, reverse transcriptase template; pegRNA, prime editing guide RNA; DNA, deoxyribonucleic acid; gDNA, genomic DNA.

FIG. 8A-C. Workflow for CAPTURE and sequence-specific adaptor ligation to RNA targets. A, Ribonucleoprotein (RNP) formation of Click Editors using an RNA-binding domain nuclease (RBDn), HUH endonuclease, and DNA-dependent polymerase complex (where the DNA polymerase can be unfused or fused to the RBDn) in combination with a guide RNA (gRNA) and clkDNA pair. B, CAPTURE workflow using adaptors that contain a 3′ overhang complementary to the adaptor-complementary overhang (ACO) region of the target RNA. C, CAPTURE workflow using oligo annealing to the RNA ACO and subsequent adaptor attachment via click chemistry. clkDNA, click DNA template oligonucleotide; cACO, complement of the ACO; FBR, flap-binding region; PT, polymerization template; RNP, ribonucleoprotein; DNA, deoxyribonucleic acid; RNA, ribonucleic acid; HUH, a histidine-hydrophobic residue-histidine (HUH) motif-containing endonuclease; RT, room temperature; dNTPs, deoxynucleoside triphosphates; ATP, adenosine triphosphate; PCR, polymerase chain reaction; CAGE, click-assisted genome enrichment; pol, polymerase.

FIG. 9A-C. Workflow for PE-CAPTURE and sequence-specific adaptor ligation to RNA targets. A, Ribonucleoprotein (RNP) formation of Prime Editors using an RNA-binding domain nuclease (RBDn) and RNA-dependent polymerase (e.g. reverse transcriptase (RT); where the RT can be unfused or fused to the RBDn) in combination with a prime editing guide RNA (pegRNA) and click DNA (clkDNA) pair. B, PE-CAPTURE workflow using adaptors that contain a 3′ overhang complementary to the adaptor-complementary overhang (ACO) region of the target RNA. C, PE-CAPTURE workflow using oligo annealing to the ACO and subsequent adaptor attachment via click chemistry. cACO, complement of the ACO; PBS, primer binding site; RTT, reverse transcriptase template; RNA, ribonucleic acid.

FIGS. 10A-D. Exemplary methodological flowcharts for CAGE and CAPTURE workflows. A, Exemplary CAGE workflow using adaptors with ACO-complementary 3′ overhangs. B, Exemplary CAGE workflow using oligo ligation and click-chemistry adaptor attachment. C, Exemplary CAPTURE workflow using adaptors with ACO-complementary 3′ overhangs. D, CAPTURE workflow using oligo ligation and click-chemistry adaptor attachment. For panels A-D, boxed steps in the flowchart indicate potential user intervention. ACO, adaptor-complementary overhang; sgRNA, single guide RNA; pegRNA, prime editing guide RNA; clkDNA, click DNA template oligonucleotide; CAGE, click-assisted genome enrichment; CAPTURE, click-assisted precise targeting of unaltered RNA for enrichment; RNP, ribonucleoprotein; DBD, DNA binding domain; RBD, RNA binding domain; PE, prime editor; DNA, deoxyribonucleic acid; RNA, ribonucleic acid; HUH, a histidine-hydrophobic residue-histidine (HUH) motif-containing endonuclease. dNTPs, deoxynucleoside triphosphates; ATP, adenosine triphosphate.

FIGS. 11A-B. CAGE reactions on PCR substrates with and without a tethering domain. CAGE on a PCR substrate containing a HEK3 Cas9 sgRNA target sequence either with (A) PCV2-Cas9 or (B) Cas9. Reactions were done with a range of clkDNA concentrations (0.1 μM to 10 μM) and a clkDNA containing a 15 bp cACO and a 17 bp PBS. The clkDNA also contained or lacked a PCV2 recognition sequence. ACO writing percentage, measured by NGS, is shown for various lengths of ACO installed where 9, 11, and 13 bp represent truncations and 15 bp represents full ACO addition.

FIG. 12. CAGE reactions on gDNA substrate with and without a tethering domain. CAGE on human (HEK293T) gDNA using an sgRNA targeting HEK3. Reactions were done with a range of clkDNA amounts (1 pmol, 5 pmol, or 10 pmol) and a clkDNA containing a 15 bp cACO, a 17 bp PBS, and a 13 bp PCV2 sequence. ACO writing percentage, measured by NGS, is shown—accounting for reads containing the 15 bp ACO with no additional PCV2 sequence installation and reads with the ACO and 4, 9, or 13 bp of the PCV2 sequence.

### DETAILED DESCRIPTION

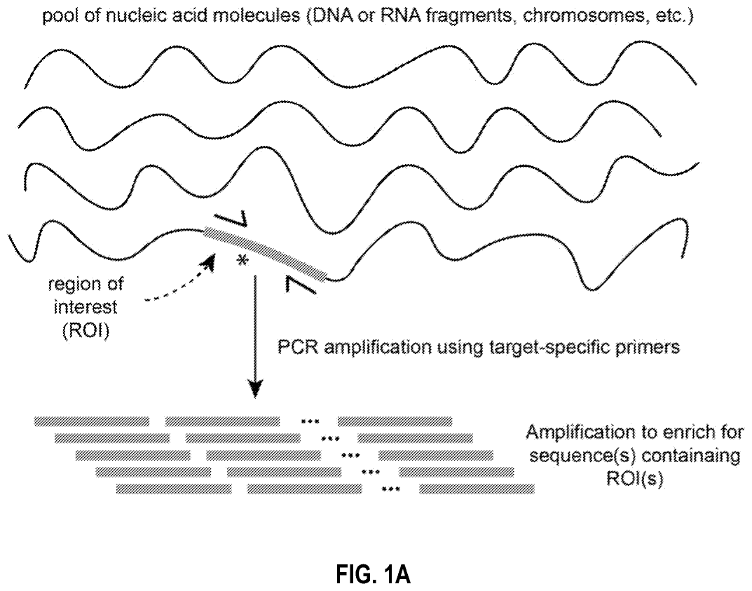

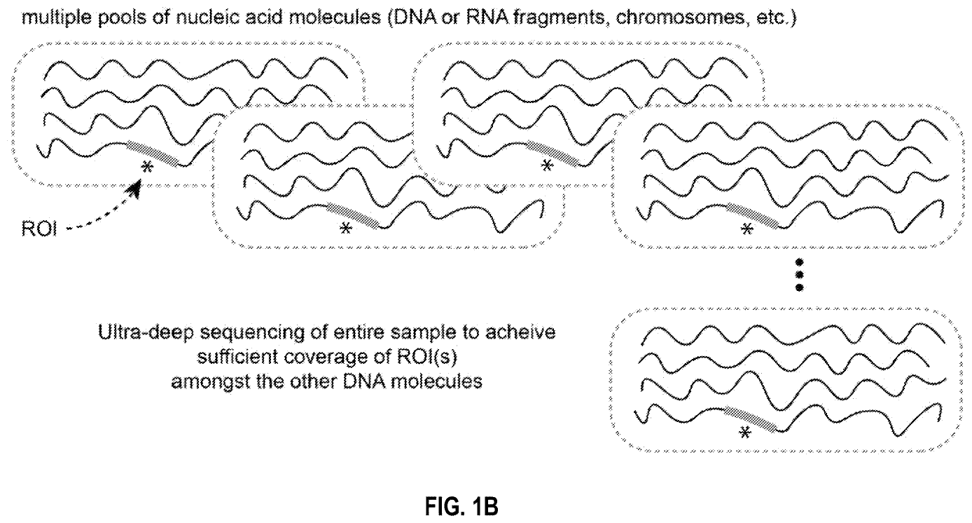

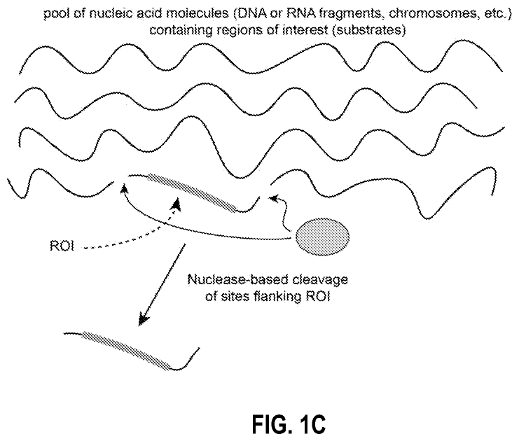

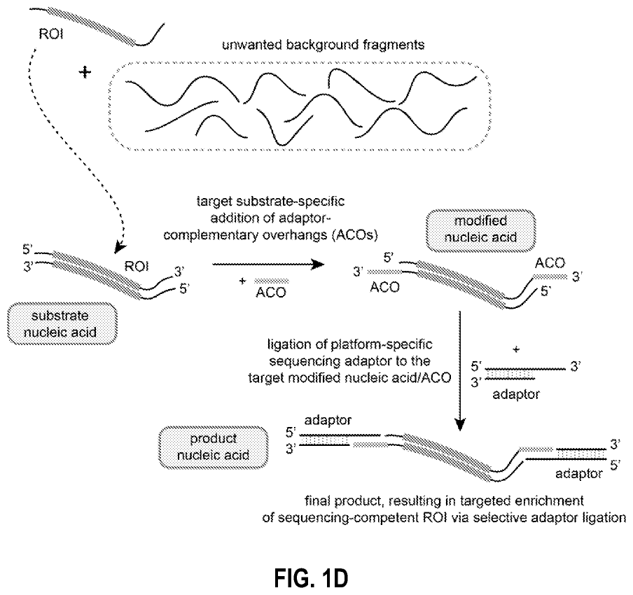

Targeted nucleic acid enrichment methods enable users to selectively sequence only regions of interest, reducing cost and labor while increasing throughput, sequence coverage, and resolution. Typically, PCR-based amplification is employed for target enrichment. However, PCR amplification introduces noise from low-level polymerase error and bias, particularly on repetitive/complex templates or for applications that have varying template sizes). Furthermore, PCR-based methods may be unfeasible for certain template compositions, and they eliminate native base modifications that are crucial to certain applications. Other methods for amplification-free target enrichment typically include the use of enzymes to selectively modify or cleave the target region⁵⁻⁷ (e.g., whole-sample dephosphorylation followed by target-specific exposure of 5′-phosphates following enzymatic cleavage by a restriction enzyme of CRISPR-Cas nuclease⁷; FIG. 1C). However, these methods often lack adequate target-molecule selectivity, require protracted protocols, exhibit inadequate background reduction (e.g. due to incomplete whole-sample dephosphorylation) that results in only about 0.5-5% target enrichment⁵⁻⁷, and/or necessitate the use of high quantities of input DNA, that together lead to low-to-moderate overall enrichment with high residual unwanted ‘background’ sequencing of non-target molecules. Thus, there is a need for methods that can significantly reduce cost by maximizing target region enrichment and sequencing coverage. Ideally, an enrichment workflow to capture only the intended nucleic acid regions prior to sequencing would have the following properties; it would: be simple, require minimal user intervention, contain no amplification steps, be compatible with a range of nucleic acid inputs, and facilitate near-100% target molecule enrichment prior to sequencing (e.g. via selective adaptor ligation to target fragment(s)).

Here we describe methods that can dramatically reduce the cost of sequencing while preserving native samples by enabling amplification-free selective enrichment of target molecules. A key premise of these approaches is to selectively cleave the substrate nucleic acid molecule(s) of interest followed by targeted polymerization of an adaptor-complementary overhang (ACO) sequence onto this sequence to create a modified nucleic acid; the sequence complementary to the ACO (cACO) is encoded on a click DNA (clkDNA) oligonucleotide template (FIG. 1D). The sequence of the ACO is user-specifiable and can be comprised of a variety of different lengths and sequence compositions, e.g., at least 1, 2, 3, 4, 5, 6, 7, 8, 9, or 10 nt and up to 10, 15, 20, 25, 30, 40, 50, 75, or 100 or more nt long, and ranges having any of the foregoing values as end points. The modified nucleic acid(s) or regions-of-interest (ROIs) comprising the ACO can then be subjected to downstream reactions that generate sequencing-competent product nucleic acid molecules; for instance, by ligation of a platform-specific sequencing adaptor where the adaptor has an overhang complementary to the ACO (e.g., for Illumina short-read or long-read sequencing, Oxford Nanopore Technologies (ONT) long-read sequencing, or Pacific Biosciences (PacBio) long-read sequencing). The selective addition of custom adaptor-complementary overhangs onto the modified target nucleic acid should virtually eliminate the background inherent to other enrichment methods, leading to maximal sequencing bandwidth and product molecule coverage. This method is compatible with any short- and long-read sequencing platform using appropriate adaptors.

Thus, provided herein are methods of using a nuclease with a DNA or RNA template (either a click editor or prime editor) and a DNA- or RNA-dependent polymerase to install a DNA or RNA 3′ overhang onto a target DNA or RNA molecule, referred to herein as CAGE and CAPTURE, using various polymerases.

The methods can include installing a 3′ ACO onto a substrate nucleic acid where the ACO has complementarity to a sequencing adaptor, or ligating an oligo with a click chemistry moiety and using an adaptor with a compatible click chemistry moiety to attach an adaptor.

Any ACO sequence or adaptor sequence can be used that is compatible with any sequencing platform.

In addition, the adaptors can be modified with a moiety that can enable pull down of adaptor-ligated molecules from non-adaptor-ligated molecules (e.g., biotin on adaptor, then use streptavidin beads in final purification step); alternatively, oligo-conjugated beads can be used to pull down product nucleic acids with an ACO installed.

### Click-Assisted Genome Enrichment (CAGE)/Click-Assisted Precise Targeting of Unaltered RNA for Enrichment (CAPTURE)

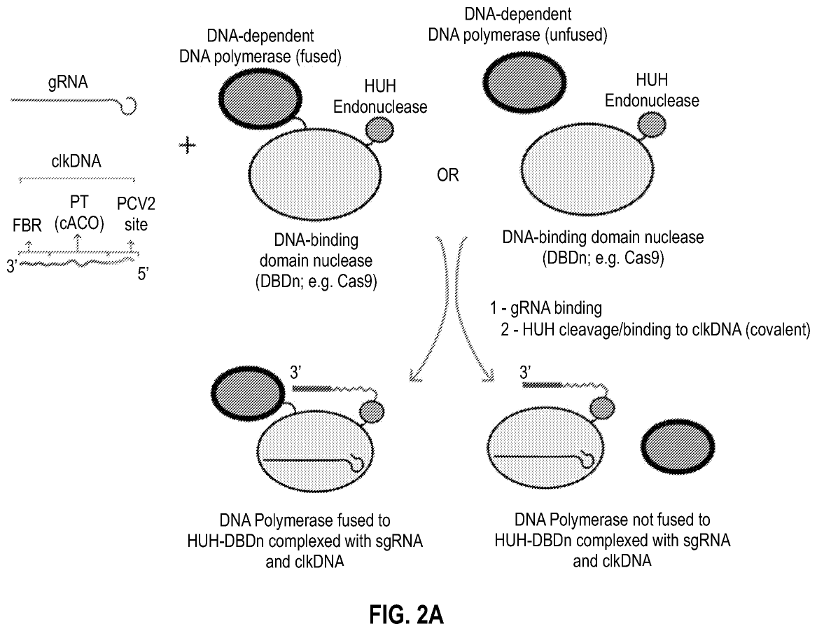

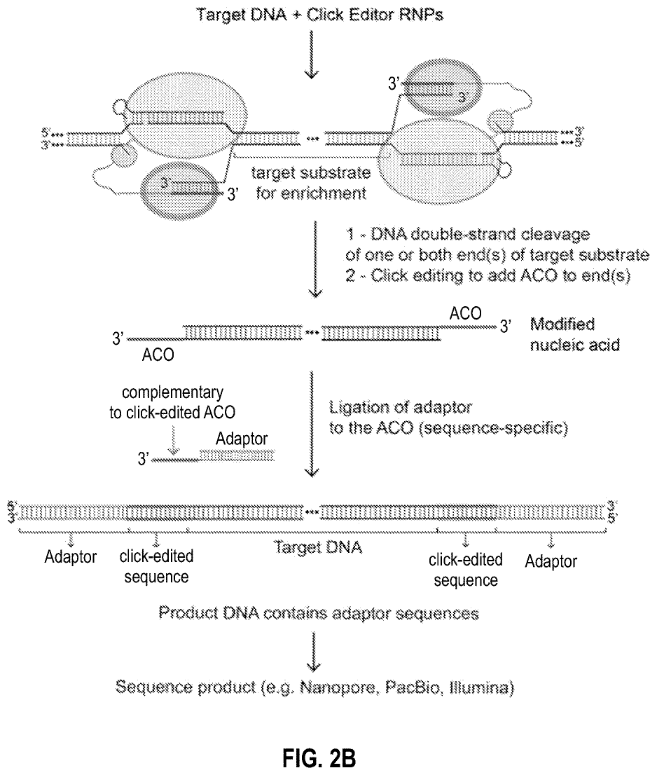

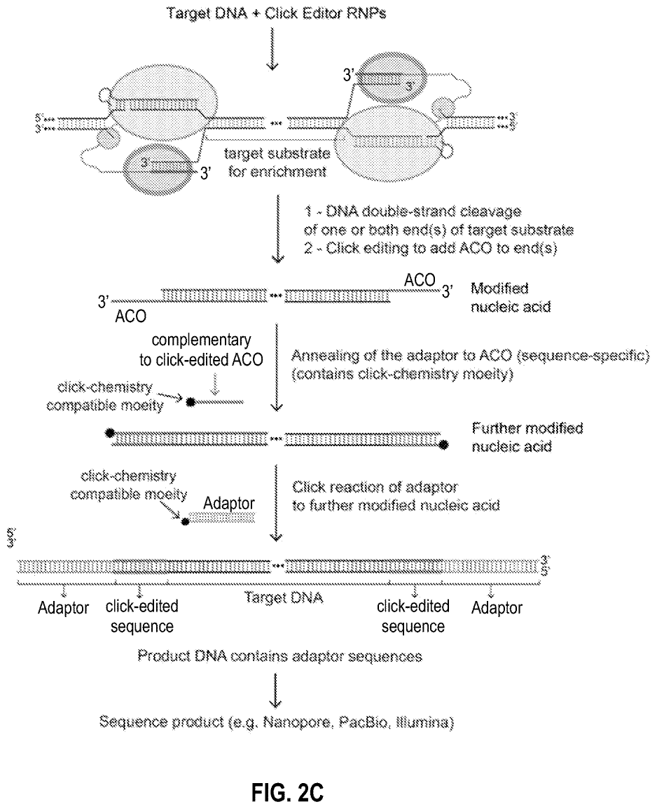

The present inventors recently described the development of click editors (CEs), a new set of technologies that enable the installation of a range of different DNA substrate edits or modifications in cells for genome editing, for eventual use in vivo, or for various uses in vitro. Exemplary polymerase click editors (PCEs) couple HUH endonucleases with RNA-guided DNA binding domains and DNA polymerases (where the DNA polymerase is either fused or supplied in trans), that be optionally directed to a target site via a guide RNA (gRNA) (FIG. 2A). In PCEs where the DNA binding domain is a CRISPR-Cas9 nickase, the PCE-gRNA complex nicks the non-target DNA strand (NTS) of a target site (within a chromosome/genome), exposing the NTS DNA ‘flap’ for modification. During this reaction, a click DNA (clkDNA) template is provided in trans; the clkDNA template encodes an HUH endonuclease recognition site (e.g., a PCV2 recognition site), a polymerization template (PT), and a NTS flap binding region (FBR); FIG. 2A. The HUH enzyme of the PCE cleaves and covalently binds to the clkDNA, permitting local recruitment and annealing of the FBR of the clkDNA with the nicked NTS of the target site, acting as a DNA:DNA duplex to recruit the DNA-dependent DNA polymerase (which can be PCE-fused or unfused) to initiate extension of the NTS using the PT portion of the clkDNA as a template. This process enables the selective writing of custom 3′ flaps into regions of genomic DNA in human cells, which through subsequent DNA repair and removal of the native 5′ flap leads to permanent installation of clkDNA-encoded sequence alterations. Flaps are the strands exposed after the DNA is cut by the nuclease; for Cas9-gRNA bound target DNAs, the 3′ flaps on the non-target strand are extended by the polymerase, so 3′ flaps are the new sequence written in from the polymerase domain of the PCE.

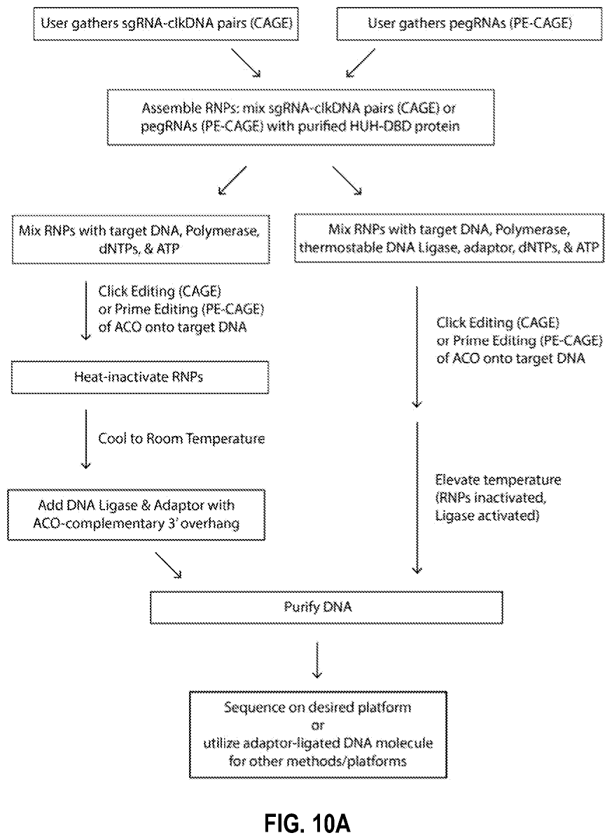

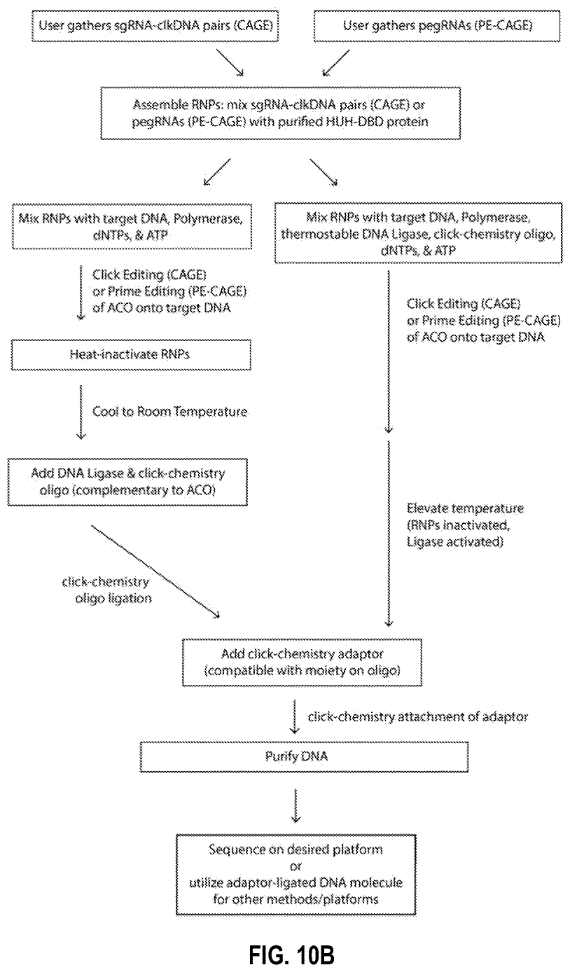

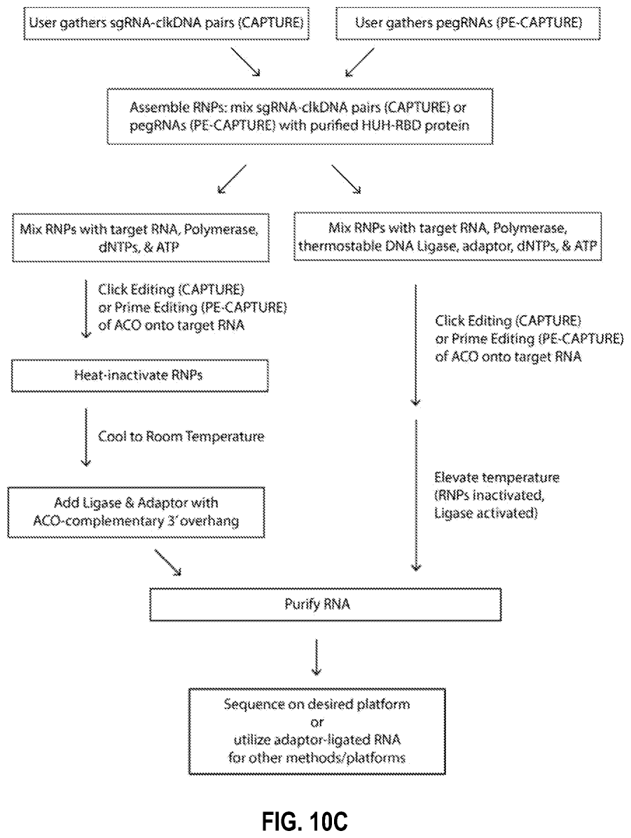

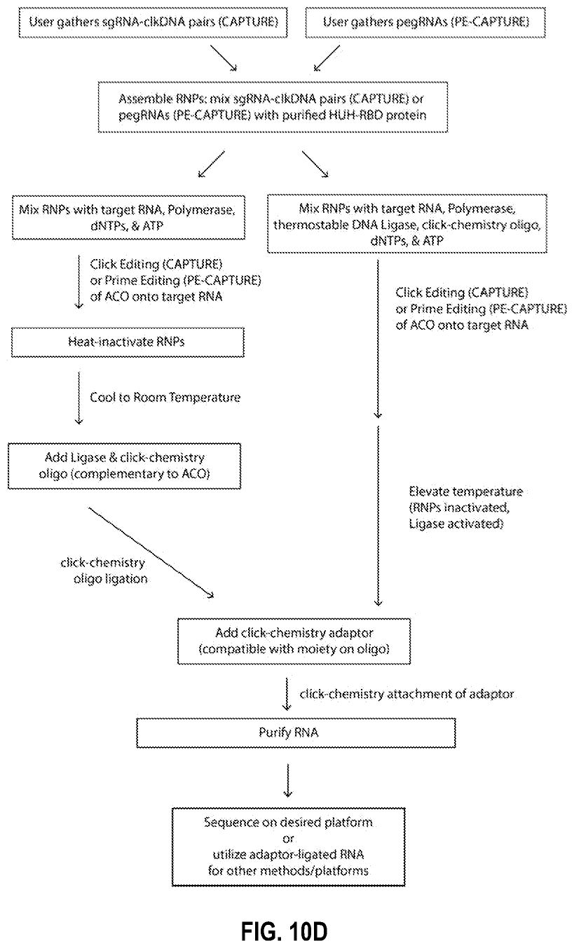

Beyond the use of PCEs in cells, modified versions of PCEs can be deployed for a variety of uses in vitro. For instance, PCEs that are comprised of nucleases instead of nickases could enable simultaneous substrate cleavage and ‘tagging’ of user-specifiable sequences. And since the stoichiometries of different reaction components are easily titratable in vitro, the polymerase can be decoupled/unfused from the HUH-Cas9 fusion. The general principles of this approach are: (1) user-specifiable sequence-specific nucleic acid cleavage (e.g., via a nuclease-active PCE construct comprising a CRISPR-Cas nuclease directed by a gRNA), (2) direct writing of customizable adaptor-complementary 3′ ‘overhang’ (ACO) sequences onto the substrate nucleic acids of interest (e.g., in a DNA version of this method via a DNA-dependent DNA polymerase of the PCE using the clkDNA as a template), where the sequence of the ACO can be of various lengths and sequence compositions, and (3) ligation of custom sequencing adaptors onto the nascent custom 3′ overhang on the modified nucleic acid (FIG. 2A). We refer to this method as Click-Assisted Genome Enrichment (CAGE); it should in principle enable near 100% amplification-free enrichment of target DNA (compared to current standards of ~0.5-5%). CAGE permits the installation of long user-specifiable 3′ overhang sequences, offering numerous advantages compared to current Cas9-based enrichment methods that rely on single nucleotide (nt) overhangs for ligation of adaptors (but due to incomplete whole-sample dephosphorylation, still leads to ~95%+ background⁷). CAGE can be used to install an overhang on a single-end of a nucleic acid (using one gRNA), or onto both ends (using two or more gRNA). While the CAGE method is applicable to DNA substrates (FIGS. 10A and 10B), the Click-Assisted Precise Targeting of Unaltered RNA for Enrichment (CAPTURE) method is applicable to RNA substrates (FIGS. 10C and 10D).

For various sequencing applications, sample multiplexing using barcodes can dramatically reduce sequencing costs by permitting the pooling of orthogonally barcoded samples into a single sequencing run. In the context of CAGE, sample multiplexing should be permitted by using adaptors that encode barcodes (as is done by various sequencing platforms including Oxford Nanopore Technologies (ONT), Pacific Biosciences (PacBio), Illumina, etc.).

CAGE can be used, e.g., for single-end labeling of DNA substrates, or dual-end labeling of DNA substrates; CAPTURE can be used for single- or dual-end labeling of RNA substrates. Single-end labeling of DNA might be preferable for quantifying or sequencing substrates with known or ambiguous ends (e.g., near the end of chromosomes or DNA fragments, for unidirectional sequencing near CRISPR-Cas genome editing target sites, sequencing from ‘bait’ sites to identify genome-scale changes, or for unbiased assessment of large sequence DNA integration or inversion events). Dual-end labeling to enrich specific sequences from certain larger DNA substrates (e.g., chromosomes) with known DNA sequences may be preferable to improve sequencing efficiency and termination.

Within the CAGE method are multiple variations that differ at the adaptor ligation step. In some embodiments, following writing of the ACO, a single adaptor molecule can be used that harbors a 3′ overhang (complementary to the ACO) and the remainder of the double-stranded region appropriate to the sequencing-platform. This single adaptor can be ligated to the ACO of the target site(s) in a single step (FIGS. 2B and 10A). In some embodiments, a two-step reaction is used. A single stranded oligonucleotide that is complementary to the ACO and that contains a 5′ modification compatible with click chemistry is annealed and ligated to the ACO/target modified nucleic acid molecule. This further modified nucleic acid is then combined in a reaction with a second adaptor containing the appropriate modification to enable click chemistry with the initial ligated oligo, facilitating the rapid attachment of adaptors to the target sequence(s) resulting in a product nucleic acid (FIGS. 2C and 10B). An example of this type of adaptor is Oxford Nanopore Technologies' “Rapid Adaptor”.

The ability to polymerize a DNA sticky-end overhang onto target DNA or RNA sequences represents a powerful approach to enable targeted, amplification-free enrichment of nucleic acid sequences in vitro for various sequencing applications and beyond. The use of click editors and prime editors of various compositions underlie CAGE and CAPTURE, making this process simple and cost-effective by requiring only simple sgRNA-clkDNA pairs or pegRNAs. These technologies and methods enable predictable and efficient overhang installation through simple single-pot reactions with only one or two steps of user-intervention (for a total of ~1-3 hrs of protocol time). CAGE offers major advantages compared to current amplification-free targeted enrichment approaches by facilitating highly specific adaptor ligation through overhang-adaptor complementarity instead of phosphorylation states⁷, reducing background in sequencing runs to enable much higher coverage and resolution at significantly lower costs. Currently, there are no comparable methods to CAPTURE for targeted, amplification-free enrichment of RNA sequences.

The applications of CAGE and CAPTURE are wide-ranging across basic biology—genomics, epigenomics, transcriptomics, epitranscriptomics, diagnostics, microbial/microbiome studies, etc. Additionally, CAGE can facilitate a more thorough investigation of intended and unintended genomic or epigenetic alterations by genome editing technologies at endogenous loci, particularly as new genome editing technologies capable of larger sequence edits continue to emerge (e.g. insertions, deletions, inversions, etc.). Similarly, CAPTURE could also be used to profile the potential of RNA editing technologies to edit the transcriptome or alter RNA modifications.

In principle, CAGE enables at or near 100% enrichment (percent of sequencing reads that are expected to be attributable to the desired target region) of target sequences because this method relies on highly specific adaptor ligation, and only adaptor-ligated product nucleic acids are sequenced (whereas other Cas9-based approaches achieve only 0.5-5% enrichment due to inherent non-specific adaptor ligation to non-target molecules⁵⁻⁷). Even with 10-30% ligation efficiency of the adaptor to the ACO-extended region-of-interest, only the adaptor-ligated molecules should be sequenced (resulting in at or near 100% enrichment). Notably, adaptor ligation efficiency dictates required DNA input amount and does not determine enrichment. Improvements to the ACO and ligation efficiency will facilitate compatibility with low and ultra-low input samples (e.g. varying the ACO sequence composition and length, inclusion of other factors like single-strand binding protein (SSB) to the reaction, etc.). Furthermore, the adaptor itself can be customized to be compatible with a broad range of sequencing platforms or for other uses, as well as modified to facilitate further physical isolation of adaptor-ligated products from non-adaptor-ligated molecules (e.g. adaptors containing a functional group like a biotin moiety, that would permit adaptor-ligated molecules to be isolated or pulled down from the population of molecules using streptavidin beads).

### Architectures

The enzymes or enzyme fusions used in CAGE and CAPTURE reactions can comprise a variety of architectures, including a domain for tethering (T) the template (also referred to herein as clkDNA tethering domain), a DNA binding domain nuclease (DBDn) for cleaving the substrate, and a polymerase (P) for extending the template, separated by optional linkers (L). Although herein we utilized a tethering-linker-nuclease with polymerase in trans architecture (T-L-DBDn+P), where the tethering domain (for template recruitment) was the HUH enzyme PCV2 and the DNA binding domain nuclease (DBDn) was SpCas9, alternate orientations could also include the optional fusion of a polymerase. Examples of alternate architectures beyond T-L-DBDn+P include but are not limited to: DBDn-L-T+P, T-L-DBDn-L-P, T-L-P-L-DBDn, DBDn-L-T-L-P, DBDn-L-P-L-T, P-L-DBDn-L-T, or P-L-T-L-DBDn (where DBDn is the nuclease, T is the tethering domain, P is the polymerase, and L is a linker; each of varying compositions). The nuclease domain (DBDn) may be SpCas9 or other nucleases; the tethering domain (T) may be an HUH enzyme (e.g. PCV2 or others) or other tethering domains; the polymerase domain (P) may be E. coli Klenow fragment or other polymerases; and the linkers (L) may be of various lengths and amino acid compositions.

Thus the DNA polymerase can be either fused or unfused from the DBDn-T complex; in addition, the T (tethering domain) can also be recruited or linked to the DBDn via other methods instead of direct fusion, including but not limited to recruitment through the gRNA; if any component is unfused, that component can optionally be recruited or linked to the target site through a protein recruitment domain, e.g., phage coat proteins (CP) (coupled with sgRNAs encoding the corresponding RNA recognition hairpin recognized by a given coat protein).

Thus, one method to selectively recruit and link proteins or domains to specific target sites is to fuse or recruit the effector protein-of-interest (e.g., polymerase and/or HUH endonuclease or other tethering domain) to a recruitment domain (e.g., an RNA binding protein, e.g., MCP, PCP, or Com RNA binding protein^{41,42}), e.g., the MS2-coat protein (MCP)) that then interacts with a specific hairpin sequence encoded within that gRNA (e.g., viral RNA sequences MS2, PP7, and com, e.g., an MS2 hairpin). This permits selective recruitment of the effector to the site bound by the primary gRNA; for example, the gRNA targeting the secondary nicking site would not harbor an MS2 hairpin, preventing recruitment to that site.

To recruit proteins to the target site, protein complexes can be formed using a protein recruitment domain coiled-coil (CC) protein domains, leucine zippers (LZs), or SunTags, that permit protein:protein interactions (among other types of protein recruitment strategies) can be used. Coiled-coil domains are known in the art, see, e.g., Woolfson, Adv Protein Chem. 2005; 70:79-112 (design of coiled-coil structures and assemblies); Grigoryan and Keating, Curr Opin Struct Biol. 2008 August; 18(4):477-83 (structural specificity in coiled-coil interactions); Reinke et al., Am. Chem. Soc. 2010, 132, 17, 6025-6031 (synthetic coiled-coil interactome, heterospecific modules for molecular engineering); Ljubetic et al., Nature Biotechnology 35:1094-1101 (2017)(coiled-coil protein-origami cages that self-assemble in vitro and in vivo); Fink et al., Nature Chemical Biology 15:115-122 (2019)(orthogonal CC dimerizing domains); Lebar et al., Nature Chemical Biology 16:513-519 (2020) (orthogonal coiled-coil domains); Plaper et al., Scientific Reports 11: 9136 (2021)(coiled-coil heterodimers); and Lainscek et al., Nature Communications 13:3604 (2022)(coiled-coil heterodimer-based recruitment of an exonuclease to CRISPR/Cas). Exemplary coiled-coil sequences include the following:

| | AA Sequence of Exemplary | SEQ ID |
|---|---|---|
| Name | Coiled-Coil Domain | NO(s): |
| P1 | EIQALEE ENAQLEQ ENAALEE EIAQLEY | 1 |
| P2 | KIAQLKE KNAALKE KNQQLKE KIQALKY | 2 |
| P3 | EIQQLEE EIAQLEQ KNAALKE KNQALKY | 3 |
| P4 | KIAQLKQ KIQALKQ ENQQLEE ENAALEY | 4 |
| P3S | EIQQLEE EISQLEQ KNSQLKE KNQQLKY | 5 |
| P4S | KISQLKQ KIQQLKQ ENQQLEE ENSQLEY | 6 |
| P5 | ENAALEE KIAQLKQ KNAALKE EIQALEY | 7 |
| P6 | KNAALKE EIQALEE ENQALEE KIAQLKY | 8 |
| P7 | EIQALEE KNAQLKQ EIAALEE KNQALKY | 9 |
| P8 | KIAQLKE ENQQLEQ KIQALKE ENAALEY | 10 |
| P9 | ENQALEQ KNAQLKQ EIAALEQ EIAQLEY | 11 |
| P10 | KNAQLKE ENAALEE KIQQLKE KIQALKY | 12 |
| P11 | ENQALEQ EIAQLEQ EIAALEQ KNAQLKY | 13 |
| P12 | KNAQLKE KIAALKE KIQQLKE ENQALEY | 14 |
| N5 | EIAALEA KIAALKA KNAALKA EIAALEA | 15 |
| N6 | KIAALKA EIAALEA ENAALEA KIAALKA | 16 |
| AP4 | ELAANEE ELQQNEQ KLAQIKQ KLQAIKY | 17 |

Exemplary combinations of CC domains include P1:P2; P3:P4; P3:P4S; P3S:P4; P3S:P4S; P5:P6; P7:P8; P9:P10; P11:P12; P3:P4; N5:N6; P3:AP4; and P3S:P4S.

Leucine zippers (LZs) are also known in the art. See, e.g., Amoutzias et al., Trends Biochem Sci. 2008 May; 33(5):220-9; Bader and Vogt, (2006). Leucine Zipper Transcription Factors: bZIP Proteins. In: Encyclopedic Reference of Genomics and Proteomics in Molecular Medicine. Springer, Berlin, Heidelberg. doi.org/10.1007/3-540-29623-9_2180; and Busch and Sassone-Corsi, Trends Genet. 1990 February; 6(2):36-40 (see, e.g., exemplary LZ domain sequences in FIG. 1 of this paper; examples include: GCN4, yAP-1, C/EBP, CREB, CRE-BP1, c-Jun, JunB, JunD, FosB, Fra-1, and c-Fos).

SunTags are described in Tanenbaum et al., Cell. 2014 Oct. 23; 159(3):635-46. Exemplary sequences include: GCN4: LLPKNYHLENEVARLKKLVGER (SEQ ID NO: 18); GCN4 variant: EELLSKNYHLENEVARLKK (SEQ ID NO: 19); and ScFv-GCN4: GPDIVMTQSPSSLSASVGDRVTITCRSSTGAVTTSNYASWVQEKPGKLFKGLI GGTNNRAPGVPSRFSGSLIGDKATLTISSLQPEDFATYFCALWYSNHWVFGQ GTKVELKRGGGGSGGGGSGGGGSSGGGSEVKLLESGGGLVQPGGSLKLSCA VSGFSLTDYGVNWVRQAPGRGLEWIGVIWGDGITDYNSALKDRFIISKDNGK NTVYLQMSKVRSDDTALYYCVTGLFDYWGQGTLVTVSS (SEQ ID NO: 20).

clkDNA Tethering Domain

The clkDNA tethering domain can include an HUH endonuclease (see, e.g., Table A) or a different ssDNA localization moiety, such as avidin (when the clkDNA is labeled with biotin), SNAP-tag (when the clkDNA is labeled with benzylguanine derivatives), CLIP-tag (when the clkDNA is labeled with benzylcytosine derivatives) (or other O⁶-alkylguanine-DNA-alkyltransferase derivatives) and HALO-tag or other haloalkane dehalogenase derivatives (when the clkDNA is labeled with a chloroalkane) (Table B).³⁴ In some embodiments, HUH endonucleases are preferred as they do not require specialized and expensive chemical modifications on the clkDNA and have high efficient covalent binding to their ssDNA substrate. Exemplary HUH endonucleases (Table A) include PCV2 HUH domain; DCV HUH domain; FBNYV HUH domain; RepBm HUH domain; TraI relaxase domain; dPCV2 (Y96F) HUH domain, MSMV HUH domain, TGMV HUH domain, ChiSCV-GT306, ChiSCV-GM510, ChiSCV-GM415, and other HUH domains described in Li, L. et al¹⁵.

TABLE A
Exemplary HUH endonuclease amino acid sequences

| | | target |
|---|---|---|
| name | aa sequence | sequence* |
| PCV2 | MSPSKKNGRSGPQPHKRWVFTLNNPSEDER | CTGTAAGTATTA |
| HUH | KKIRDLPISLFDYFIVGEEGNEEGRTPHLQGFA | CCAGC (SEQ |
| domain | NFVKKQTFNKVKWYLGARCHIEKAKGTDQQ | ID NO: 41) |
| | NKEYCSKEGNLLMECGAPRSQGQR (SEQ ID | |
| | NO: 21) | |
| DCV | MAKSGNYSYKRWVFTINNPTFEDYVHVLEFC | GTTATATTATTA |
| HUH | TLDNCKFAIVGEEKGANGTPHLQGFLNLRSNA | CCGGC (SEQ |
| domain | RAAALEESLGGRAWLSRARGSDEDNEEYCAK | ID NO: 42) |
| | ESTYLRVGEPVSKGRSSDLAEATSAV (SEQ | |
| | ID NO: 22) | |
| FBNYV | MARQVICWCFTLNNPLSPLSLHDSMKYLVY | GGCTTAGTATTA |
| HUH | QTEQGEAGNIHFQGYIEMKKRTSLAGMKKL | CCCCC (SEQ |
| domain | IPGAHFEKRRGTQGEARAYSMKEDTRLEGP | ID NO: 43) |
| | WEYGEFVP (SEQ ID NO: 23) | |
| RepBm | MSEKKEIVKGRDWTFLVYPESAPENWRTILDE | TGCTTCCGTACT |
| HUH | TFMRWVESPLHDKDVNADGEIKKPHWHILLS | ACGACCCCCCA |
| domain | SDGPITQTAVQKIIGPLNAPNAQKVGSAKGLV | (SEQ ID NO: |
| | RYMVHLDNPEKYQYSLDEIVGHNGADVASY | 44) |
| | FELTA (SEQ ID NO: 24) | |
| TraI | MMSIAQVRSAGSAGNYYTDKDNYYVLGSMG | TTTGCGTGGGGT |
| relaxase | ERWAGRGAEQLGLQGSVDKDVFTRLLEGRLP | GTGGTGCT |
| domain | DGADLSRMQDGSNRHRPGYDLTFSAPKSVSM | (SEQ ID NO: |
| | MAMLGGDKRLIDAHNQAVDFAVRQVEALAST | 45) |
| | RVMTDGQSETVLTGNLVMALFNHDTSRDQEP | |
| | QLHTHAVVANVTQHNGEWKTLSSDKVGKTGF | |
| | IENVYANQIAFGRLYREKLKEQVEALGYETE | |
| | VVGKHGMWEMPGVPVEAFSGRSQTIREAV | |
| | GEDASLKSRDVAALDTRKSKQHVDPEIKMAEW | |
| | MQTLKETGFDIRAYRDAADQRADLRTLTPGPA | |
| | SQDGPDVQQAVTQAIAGLSER (SEQ ID NO: | |
| | 25) | |
| dPCV2 | MSPSKKNGRSGPQPHKRWVFTLNNPSEDERK | CTGTAAGTATTA |
| (Y96F) | KIRDLPISLFDYFIVGEEGNEEGRTPHLQGFA | CCAGC (SEQ |
| HUH | NFVKKQTFNKVKWYLGARCHIEKAKGTDQQNK | ID NO: 41) |
| domain | EFCSKEGNLLMECGAPRSQGQR (SEQ ID NO: | |
| | 26) | |
| RepB | MAKEKARYFTFLLYPESIPSDWELKLETLGVPMA | Tgcttccgtacta |
| HUH | ISPLHDKDKSSIKGQKYKKAHYHVLYIAKNPVTA | cgacccccca |
| domain | DSVRKKIKLLLGEKSLAMVQVVLNVENMYLYLTH | (SEQ ID NO: |
| | ESKDAIAKKKHVYDKADIKLINNFDIDRY (SEQ | 44) |
| | ID NO: 27) | |
| BBTV | MARYVVCWMFTINNPTTLPVMRDEIKYMVYQVER | GGCTTATTATTAC |
| HUH | GQEGTRHVQGYVEMKRRSSLKQMRVFFPGAHLEK | CCCC (SEQ ID |
| domain | RKGSQEEARSYCMKEDTRIEGPFEFGAFKLSCND | NO: 46) |
| | NLFDVIQDMRETHKRPLEYLYDCPNTFDRSKDTL | |
| | YRVQAELNKTRAMNSWRTSFSAWTSEVENIMAQP | |
| | CHRRIIRVYGPNGGEGKTTYAKHLMKTKNAFYSP | |
| | GGKSLDICRLYNYEDIVIFDIPRCKEDYLNYGLL | |
| | EEFKNGIIQSGKYEPVLKVVEYVEVIVMANFLPK | |
| | EGIFSEDRIKLVSC (SEQ ID NO: 28) | |
| WDV | MASSSAPRFRVYSKYLFLTYPQCTLEPQYALDSL | GTTATAATATTAC |
| HUH | RTLLNKYEPLYIAAVRELHEDGSPHLHVLVQNKL | CCCC (SEQ ID |
| domain | RASITNPNALNLRMDTSPFSIFHPNIQAAKDCNQ | NO: 47) |
| | VRDYITKEVDSDANTAEWGTFVAVSTPGRKDRDA | |
| | DMKQIIESSSSREEFLSMVCNRFPFEWSIRLKDF | |
| | EYTARHLFPDPVATYTPEFPTESLICHETIESWK | |
| | NEHLYSESPGRHKSIYICGPTRTGKTSWARSLGT | |
| | HNYYNSLVDFTTYDVNAKYNIIDDIPFKFTPNWK | |
| | CFVGAQRDFTVNPKYGKRKVIRGGIPCIILVNPD | |
| | EDWLKDMTPEQSDYMYSNAVVHYMYEGESFINYS | |
| | FASGEDVTASQ (SEQ ID NO: 29) | |
| TYLCV | MPRLFKIYAKNYFLTYPNCSLSKEEALSQLKNLET | CGTATAATATTAC |
| HUH | PTNKKYIKVCRELHENGEPHLHVLIQFEGKYQCKN | CGGA |
| domain | QRFFDLVSPNRSAHFHPNIQAAKSSTDVKTYVEKD | (SEQ ID NO: |
| | GDFIDFGVFQIDGRSARGGQQSANDAYAEALNSGN | 48) |
| | KSEALNILKEKAPKDYILQFHNLSSNLDRIFSPPL | |
| | EVYVSPFLSSSFNQVPDELEEWVAENVVSSAARPW | |
| | RPNSIVIEGDSRTGKTMWARSLGPHNYLCGHLDLS | |
| | PKVYSNDAWYNVIDDVDPHYLKHFKEFMGAQRDWQ | |
| | SNTKYGKPIQIKGGIPTIFLCNPGPTSSYREYLDE | |
| | EKNISLKNWALKNATFVTLYEPLFASINQGPTQDS | |
| | QEETNKA (SEQ ID NO: 30) | |
| CpCDV | MPSANKNFRFQSKYVFLTYPKCSSQRDDLFEFLWE | AAATTAATATTAC |
| HUH | KLTPFLIFFLGVATELHQDGTTHYHALIQLDKKPW | CGGC (SEQ ID |
| domain | IRDPSFFDFEGNHPNIQPARNSKQVLEYISKDGDI | NO: 49) |
| | KTRGDFRDHKVSPRKSDARWRTIIQTATSKEEYLD | |
| | MIKEEFPHEWATKLQWLEYSANKLFPPQPEAYVSP | |
| | FTESDLRCHEDLAQWRDTHLYQEPRRTGARVPSLY | |
| | ICGPTRTGKTTWARSLGRHNYWNGTIDFTTYDEHA | |
| | TYNVIDDIPFKFVPLWKQLIGCQFDFTVNPKYGKK | |
| | KKIKGGVPSIILTNRDEDWIPAMSEHQKEYFTDNC | |
| | EIHYMDDGETFFARESSSH (SEQ ID NO: 31) | |
| MSMV | MSHTSFRFRAKNVFLTYPRCPIGPEFLCDHLWNLV | ATAATAATATTAC |
| HUH | TPYDPLYVHVAQENHKDGGLHSHVLIQTRIEISTF | GCGC (SEQ ID |
| domain | DPTYFDYTGTSIPGAVVFHPNIQACRNVRDCLAYI | NO: 50) |
| | RKNTINEVSKGAFKCSGAGRPKKQDSAPSRDAKMC | |
| | QIMSSATSRSDYLSMVRGAFPFEWATKLAQFEYSA | |
| | SKLFPDAPTTNAIPSNIDLTCHENIMPWLRDDLYT | |
| | ERGQGRRPRSLYICGRTRTGKTTWARSLGPHNYFQ | |
| | HQVDYFAWSEEATYVVIDDIPFKFCPNWKAIVGSQ | |
| | HDYTVNPKYGKKRKLKGGIPAIILVNEDEDWLMSM | |
| | TTSQIDWFLENCVVYHMTPGESFFNYAE (SEQ | |
| | ID NO: 32) | |
| CLCV | MPRNPKSFRLAARNIFLTYPQCDIPKDEALQMLQT | GCAATAATATTAC |
| HUH | LSWSVVKPTYIRVAREEHSDGSPHLHCLIQLSGKS | CGGA (SEQ ID |
| domain | NIKDARLFDITHPRRSANFHPNIQAAKDTNAVKNY | NO: 51) |
| | ITKDGDYCESGQYKVSGGTKANKDDVYHNAVNAGC | |
| | VEEALAIIKAGDPKTFIVSYHNVRANIERLFTKAP | |
| | EPWAPPFQLSSFTNVPDEMRSWADDYFGRSAAARP | |
| | ERPISIIVEGDSRTGKTMWARALGPHNYLSGHLDF | |
| | NSKVFSNNVEYNVIDDIAPHYLKLKHWKELIGAQR | |
| | DWQSNCKYGRPVQIKGGIPSIVLCNPGEGSSYISF | |
| | LNKEENASLRAWTIKNAKFITLEAPLYQSTAQDC | |
| | (SEQ ID NO: 33) | |
| TGMV | MPSHPKRFQINAKNYFLTYPQCSLSKEESLSQLQA | CGTTTAATATTAC |
| HUH | LNTPINKKFIKICRELHEDGQPHLHVLIQFEGKYC | CGGA |
| domain | CQNQRFFDLVSPTRSAHFHPNIQRAKSSSDVKTYI | (SEQ ID NO: |
| | DKDGDTLVWGEFQVDGRSARGGCQTSNDAAAEALN | 52) |
| | ASSKEEALQIIREKIPEKYLFQFHNLNSNLDRIFD | |
| | KTPEPWLPPFHVSSFTNVPDEMRQWAENYFGKSSA | |
| | ARPERPISIIIEGDSRTGKTMWARSLGPHNYLSGH | |
| | LDLNSRVYSNKVEYNVIDDVTPQYLKLKHWKELIG | |
| | AQRDWQTNCKYGKPVQIKGGIPSIVLCNPGEGASY | |
| | KVFLDKEENTPLKNWTFHNAKFVFLNSPLYQSSTQ | |
| | SS (SEQ ID NO: 34) | |
| TrwC | MLSHMVLTRQDIGRAASYYEDGADDYYAKDGDA | GCGCACCGAAAGG |
| relaxase | SEWQGKGAEELGLSGEVDSKRFRELLAGNIGEG | TGCGTATTGTCTA |
| domain | HRIMRSATRQDSKERIGLDLTFSAPKSVSLQAL | T |
| | VAGDAEIIKAHDRAVARTLEQAEARAQARQKIQ | (SEQ ID NO: |
| | GKTRIETTGNLVIGKFRHETSRERDPQLHTHAV | 53) |
| | ILNMTKRSDGQWRALKNDEIVKRTRYLGAVYNA | |
| | ELAHELQKLGYQLRYGKDGNFDLAHIDRQQIEG | |
| | FSKRTEQIAEWYAARGLDPNSVSLEQKQAAKVL | |
| | SRAKKTSVDREALRAEWQATAKELGIDFSRREW | |
| | S (SEQ ID NO: 35) | |
| NES | MAMYHFQNKFVSKANGQSATAKSAYNSASRIKDF | Acgcgaacggaac |
| relaxase | KENEFKDYSNKQCDYSEILLPNNADDKFKDREYL | gttcgcataagtg |
| domain | WNKVHDVENRKNSQVAREIIIGLPNEFDPNSNIE | cgcccttacggga |
| | LAKEFAESLSNEGMIVDLNIHKINEENPHAHLLC | tttaac (SEQ |
| | TLRGLDKNNEFEPKRKGNDYIRDWNTKEKHNEWR | ID NO: 54) |
| | KRWENVQNKHLEKNGFSVRVSADSYKNQNIDLE | |
| | PTKKEGWKARKFEDETG (SEQ ID NO: 36) | |
| mMobA | MAIYHLTAKTGSRSGGQSARAKADYIQREGKYAR | CCAGTTTCTCGAA |
| relaxase | DMDEVLHAESGHMPEFVERPADYWDAADLYERAN | GAGAAACCGGTAA |
| domain | GRLFKEVEFALPVELTLDQQKALASEFAQHLTGA | GTGCACCCTCCC |
| | ERLPYTLAIHAGGGENPHCHLMISERINDGIERP | (SEQ ID NO: |
| | AAQWFKRYNGKTPEKGGAQKTEALKPKAWLEQTR | 55) |
| | EAWADHANRALERAGH (SEQ ID NO: 37) | |
| ChiSCV- | MHLFTDVVSVDIMTTMQRTHTNAELIRWFKIFRD | AATAGTTACGAAA |
| GM415 | LDIHKWVIGLEEGKGGYGHWQVRCNVRVEVVNDW | A |
| | TAYLRAVFGWLGPISIWTEECSDKYTYETKEGKY | (SEQ ID |
| | WASWDTMGARQQRFGKMRWNQEGAVQALQRTNDR | NO: 56) |
| | EIVVWYDEQGNMGKSWLCGHLFETGQAYYIPPYM | |
| | TSIQSMIQTVASLVLQDRDSGYPPRPLIVIDIPR | |
| | SWKWSTELYTAIEAIKDGLIMDPRYGARPVNIHG | |
| | IKVIVLTNTKPKLDKLSEDRWVLYDPMDYLMML | |
| | (SEQ ID NO: 38) | |
| ChiSCV- | MHLFTEGISVDIMTTMQRTHTNAELIRWFKIFRDL | AATAGTTACGAAA |
| GM510 | DIHKWVIGLEEGKGEYGHWQVRCNVRVEVVNDWTA | A (SEQ ID NO: |
| | YLRAVFGWLGPISIWTEECSDKYTYETKEGKYWAS | 56) |
| | WDTMGARQQRFGKMRWNQEGAVQALQRTNDREIV | |
| | VWYDEGGNMGKSWLCGHLFETGQAYYIPPYMTSI | |
| | QSMIQTVASLVLQDRETGYPPRPLIVIDIPRSWK | |
| | WSTELYTAIEAIKGGLIMDPRYGARPVNIHGTKV | |
| | IVLTNTKPKLDKLSEDRWVLYDPMDYLMML | |
| | (SEQ ID NO: 39) | |
| ChiSCV- | MGARQQRFGKMRWNQEGALQALQRTNDREIVVWY | AATAGTTACGAAA |
| GT306 | DQDGNMGKSWLCGHLYETGQAYYIPPYMTSIQSM | A (SEQ ID NO: |
| | IQTVASLVLQDRESGYPPRPLIVIDIPRSRKWST | 56) |
| | ELYTAIEAIKDGLIMDPRYGARPVNIHGTKVIVL | |
| | TNTKPKLDKLSEDRWVLYDPLLYMMGH | |
| | (SEQ ID NO: 40) | |

*nonanucleotide sequence is bolded where known, additional sequence from 5′ and 3′ stem sequence flanking nonanucleotide sequence

TABLE B
Alternative clkDNA tethering domains

| name | aa sequence | Target moiety |
|---|---|---|
| SNAP | MDKDCEMKRTTLDSPLGKLELSGCEQGLHRIIFLGKGTSAAD | benzylguanine |
| tag | AVEVPAPAAVLGGPEPLMQATAWLNAYFHQPEAIEEFPVPAL | |
| | HHPVFQQESFTRQVLWKLLKVVKFGEVISYSHLAALAGNPAA | |
| | TAAVKTALSGNPVPILIPCHRVVQGDLDVGGYEGGLAVKEWL | |
| | LAHEGHRLGKPGLG (SEQ ID NO: 57) | |
| CLIP | MDKDCEMKRTTLDSPLGKLELSGCEQGLHRIIFLGKGTSAADA | benzylcytosine |
| tag | VEVPAPAAVLGGPEPLIQATAWLNAYFHQPEAIEEFPVPALHH | |
| | PVFQQESFTRQVLWKLLKVVKFGEVISESHLAALVGNPAATAA | |
| | VNTALDGNPVPILIPCHRVVQGDSDVGPYLGGLAVKEWLLAHE | |
| | GHRLGKPGLG (SEQ ID NO: 58) | |
| HALO | MAEIGTGFPFDPHYVEVLGERMHYVDVGPRDGTPVLFLHGNPT | chloroalkane |
| tag | SSYVWRNIIPHVAPTHRCIAPDLIGMGKSDKPDLGYFFDDHVR | |
| | FMDAFIEALGLEEVVLVIHDWGSALGFHWAKRNPERVKGIAFM | |
| | EFIRPIPTWDEWPEFARETFQAFRTTDVGRKLIIDQNVFIEGT | |
| | LPMGVVRPLTEVEMDHYREPFLNPVDREPLWRFPNELPIAGEP | |
| | ANIVALVEEYMDWLHQSPVPKLLFWGTPGVLIPPAEAARLAKS | |
| | LPNCKAVDIGPGLNLLQEDNPDLIGSEIARWLSTLEISG | |
| | (SEQ ID NO: 59) | |
| mSA-H | MAEAGITGTWYNQHGSTFTVTAGADGNLTGQYENRAQGTGCQN | biotin |
| | SPYTLTGRYNGTKLEWRVEWNNSTENCHSRTEWRGQYQGGAEA | |
| | RINTQWNLTYEGGSGPATEQGQDTFTKVKPSAAS (SEQ ID | |
| | NO: 60) | |
| eMA | MFDASNFKDESSIASASSSWQNQSGSTMIIQVDSFGNVSGQYV | biotin |
| | NRAQGTGCQNSPYPLTGRVNGTFIAFSVGWNNSTENCNSATGW | |
| | TGYAQVNGNNTEIVTSWNLAYEGGSGPAIEQGQDTFQYVPTTE | |
| | (SEQ ID NO: 61) | |

mSA-H, (monomeric streptavidin);
eMA, enhanced monomeric avidin

### DNA Binding Domains

The CEs described herein optionally include an RNA-programmable DNA nickase that nicks the NTS, or a nuclease, including nickases from Cas-family enzymes (e.g., Cas9 or Cas12), TnpB-family, or IscB-family enzymes (Table C). See, e.g., Kapitonov et al., J Bacteriol. 2016 Mar. 1; 198(5): 797-807; Karvelis et al., Nature. 2021; 599(7886): 692-696 (TnpB); Koonin and Makarova, PLoS Biol. 2022 January; 20(1): e3001481; Mingarro et al., Gene, 852:147064 (2023); Altae-Tran et al., Science. 2021 October; 374(6563):57-65 (TnpB and IscB); Meers et al., bioRxiv 2023.03.14.532601 (TnpB and IscB); Schuler et al., Science. 2022 Jun. 24; 376(6600):1476-1481; Kato et al., Nat Commun. 2022 Nov. 7; 13(1):6719. Nickases can be generated from wild type RNA-programmable DNA nucleases by the introduction of a mutation of a catalytic RuvC-1 residue or a mutation of a catalytic HNH residue (Table C). For example, A. warmingii IscB nickases can include an H212A or E157A mutation; IscB nickases from other species can include corresponding mutations; see, e.g., WO 2022/087494. The nickase can also include one or more mutations that increase activity, reduce off-target effects, and/or alter protospacer adjacent motif (PAM) or target adjacent motif (TAM) specificity (Tables D and E). Exemplary Cas9 and Cas12 nickases and mutations are shown in Tables C-E.

TABLE C
List of Exemplary Cas9, Cas12a, and IscB Orthologs
(see WO2018218166 for references)

| | UniProt or | |
|---|---|---|
| | GenBank | |
| | Accession | |
| | Number | Nickase Mutations/Catalytic |
| Ortholog | aa sequence | residues* |
| S. pyogenes Cas9 | or Q99ZW2.1 | D10A (RuvC), E762A (RuvC), |
| (SpCas9) | | D839A (HNH), H840A (HNH), |
| | | N854A (HNH partial), N863A |
| | | (HNH), D983A (RuvC), |
| | | D986A (RuvC) |
| S. aureus Cas9 | J7RUA5.1 | D10A (RuvC), D556A (HNH), |
| (SaCas9) | | H557A (HNH), N571A (HNH), |
| | | N580 (HNH) |
| S. thermophilus | G3ECR1.2 | D31A (RuvC), D867A (HNH), |
| Cas9 (St1Cas9) | | H868A (HNH), N882A, (HNH), |
| | | N891A (HNH) |
| N. meningitidis | CBA04028.1 | D16A (RuvC), D587A (HNH), |
| Cas9 (NmelsCas9) | | H588A (HNH), N602A (HNH), |
| | | N611A (HNH) |
| N. meningitidis | WP_002230835.1 | D16A (RuvC),D587A (HNH), |
| DE10444 | | H588A (HNH), N602A (HNH), |
| Cas9 (Nme2Cas9) | | N611A (HNH) |
| S. pasteurianus | BAK30384.1 | D10A (RuvC), D598A (HNH), |
| Cas9 (SpaCas9) | | H599A (HNH), N613A (HNH), |
| | | N622A (HNH) |
| C. jejuni Cas9 | Q0P897.1 | D8A (RuvC), D558A (HNH), |
| (CjCas9) | | H559A (HNH), N573A (HNH), |
| | | N582A (HNH) |
| F. novicida Cas9 | A0Q5Y3.1 | D11A (RuvC), D968A (HNH), |
| (FnCas9) | | H969A (HNH), N986A (HNH), |
| | | N995A (HNH) |
| P. lavamentivorans | A7HP89.1 | D8A (RuvC), D600A (HNH), |
| Cas9 (PlCas9) | | H601A (HNH), N615A (HNH), |
| | | N624A (HNH) |
| C. lari Cas9 | G1UFN3.1 | D7A (RuvC), D566A (HNH), |
| (ClCas9) | | H567 (HNH) |
| Pasteurella | Q9CLT2.1 | D13A (RuvC), D579A (HNH), |
| multocida Cas9 | | H580A (HNH), |
| F. novicida | A0Q7Q2.1 | D917A, E1006A, D1255A (all |
| Cpfl (FnCpf1) | | RuvC) |
| M. bovoculi | WP_052585281.1 | D986A (RuvC) |
| Cpf1 (MbCpf1) | | |
| A. sp. BV3L6 | U2UMQ6.1 | D90A8, E993A, Q1226A, |
| Cpfl (AsCpf1) | | D1263A (all RuvC) |
| L. bacterium | A0A182DWE3.1 | D832A (RuvC) |
| N2006 (LbCpf1) | Sequence below | E193A (RuvC) and H247A |
| ogeulscB | | (HNH) |

*for Cas9 and IscB enzymes, the RuvC domain nicks the non-target strand (NTS) DNA and the HNH domain nicks the target strand (TS) DNA. For Cas12a/Cpf1 or TnpB enzymes, the RuvC domain nicks both DNA strands. Mutations abrogate activity.

The sequence of ogeuIscB is as follows (from metagenome genome assembly, contig: NODE_25_length_150080_coy_8.882980; contig accession: OGEU01000025.1):

(SEQ ID NO: 62)
MAVVYVISKSGKPLMPTTRCGHVRILLKEGKARVVERKPFTIQL
TYESAEETQPLVLGIDPGRTNIGMSVVTESGESVFNAQIETRNK
DVPKLMKDRKQYRMAHRRLKRRCKRRRRAKAAGTAFEEGEKQRL
LPGCFKPITCKSIRNKEARFNNRKRPVGWLTPTANHLLVTHLNV
VKKVQKILPVAKVVLELNRFSFMAMNNPKVQRWQYQRGPLYGKG
SVEEAVSMQQDGHCLFCKHGIDHYHHVVPRRKNGSETLENRVGL
CEEHHRLVHTDKEWEANLASKKSGMNKKYHALSVLNQIIPYLAD
QLADMFPGNFCVTSGQDTYLFREEHGIPKDHYLDAYCIACSALT
DAKKVSSPKGRPYMVHQFRRHDRQACHKANLNRSYYMGGKLVAT
NRHKAMDQKTDSLEEYRAAHSAADVSKLTVKHPSAQYKDMSRIM
PGSILVSGEGKLFTLSRSEGRNKGQVNYFVSTEGIKYWARKCQY
LRNNGGLQIYV

TABLE D
List of Exemplary High Fidelity and/or PAM-relaxed RGN Orthologs

| Published HF/ | | |
|---|---|---|
| PAM-RGN variants | PMID | Mutations* |
| S. pyogenes Cas9 | 26628643 | K810A/K1003A/R1060A eSpCas9(1.0); |
| (SpCas9) eSpCas9 | | K848A/K1003A/R1060A eSpCas9(1.1) |
| S. pyogenes Cas9 | 26735016 | N497A/R661A/Q695A/Q926A (SpCas9-HF1); L169A, |
| (SpCas9) HF1 | | Y450A, |
| S. pyogenes Cas9 | 28931002 | N692A, M694A, Q695A, H698A |
| (SpCas9) HypaCas9 | | |
| S. pyogenes Cas9 | 29431739 | M495V/Y515N/K526E/R661Q; |
| (SpCas9) evoCas9 | | (M495V/Y515N/K526E/R661S; |
| | | M495V/Y515N/K526E/R661L) |
| S. pyogenes Cas9 | 30082871 | R691A |
| (SpCas9) HiFi Cas9 | | |
| S. pyogenes Cas9 | 30082838 | F539S, M763I, K890N |
| (SpCas9) Sniper- | | |
| Cas9 | | |
| S. pyogenes Cas9 | 32144253 | Replacement of L1004-K1014 with two glycines |
| (SpCas9) Blackjack | | |
| S. pyogenes Cas9 | 32187529 | N690C, T769I, G915M, N980K |
| (SpCas9) LZ3 | | |
| S. pyogenes Cas9 | 35236982 | Y1010D, Y1013D, Y1016D, V1018D, R1019D, |
| (SpCas9) SuperFi- | | Q1027D, K1031D |
| Cas9 | | |
| S. pyogenes Cas9 | 36894722 | E1007L |
| (SpCas9) Sniper2L | | |
| S. pyogenes Cas9 | 36894722 | E1007P |
| (SpCas9) Sniper2P | | |
| S. pyogenes Cas9 | 36905119 | K526D |
| (SpCas9) rCas9HF | | |
| S. pyogenes Cas9 | 29512652 | A262T, R324L, S409I, E480K, E543D, M694I, |
| (SpCas9) xCas9 | | E1219V |
| S. pyogenes Cas9 | 30166441 | R1335V,L1111R,D1135V,G1218R, |
| (SpCas9) SpCas9- | | E1219F,A1322R,T1337R |
| NG | | |
| S. pyogenes Cas9 | 26098369 | D1135V,R1335Q,T1337R (SpCas9-VQR); |
| (SpCas9) | | D1135V/G1218R/R1335E/T1337R (SpCas9-VRER); |
| VQR/VRER | | D1135E |
| S. pyogenes Cas9 | 26098369, | D1135V,G1218R,R1335Q,T1337R |
| (SpCas9) SpCas9- | 26735016 | |
| VRQR | | |
| S. aureus Cas9 | 26524662 | E782K/N968K/R1015H |
| (SaCas9)-KKH | | |
| enAsCas12a | USSN | One or more of : E174R, S170R, S542R, K548R, |
| | 15/960,271 | K548V, N551R, N552R, K607R, K607H, e.g., |
| | | E174R/S542R/K548R, E174R/S542R/K607R, |
| | | E174R/S542R/K548V/N552R, S170R/S542R/K548R, |
| | | S170R/E174R, E174R/S542R, S170R/S542R, |
| | | E174R/S542R/K548R/N551R, E174R/S542R/K607H, |
| | | S170R/S542R/K607R, or |
| | | S170R/S542R/K548V/N552R |
| enAsCas12a-HF | USSN | One or more of : E174R, S542R, K548R, e.g., |
| | 15/960,271 | E174R/S542R/K548R, E174R/S542R/K607R, |
| | | E174R/S542R/K548V/N552R, S170R/S542R/K548R, |
| | | S170R/E174R, E174R/S542R, S170R/S542R, |
| | | E174R/S542R/K548R/N551R, E174R/S542R/K607H, |
| | | S170R/S542R/K607R, or |
| | | S170R/S542R/K548V/N552R, with the addition of one |
| | | or more of: N282A, T315A, N515A and K949A |
| enLbCas12a(HF) | USSN | One or more of T152R, T152K, D156R, D156K, |
| | 15/960,271 | Q529K, G532R, G532K, G532Q, K538R, K538V, |
| | | D541R, Y542R, M592A, K595R, K595H, K595S or |
| | | K595Q, e.g., D156R/G532R/K538R, |
| | | D156R/G532R/K595R, D156R/G532R/K538V/Y542R, |
| | | T152R/G532R/K538R, T152R/D156R, D156R/G532R, |
| | | T152R/G532R, D156R/G532R/K538R/D541R, |
| | | D156R/G532R/K595H, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| en FnCas12a(HF) | USSN | One or more of T177A, K180R, K180K, E184R, |
| | 15/960,271 | E184K, T604K, N607R, N607K, N607Q, K613R, |
| | | K613V, D616R, N617R, M668A, K671R, K671H, |
| | | K671S, or K671Q, e.g., E184R/N607R/K613R, |
| | | E184R/N607R/K671R, E184R/N607R/K613V/N617R, |
| | | K180R/N607R/K613R, K180R/E184R, E184R/N607R, |
| | | K180R/N607R, E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301A, K589A, |
| | | N580A, K962A, S334A, K320A, K978A |
| chimeric Cas9 | 30718489 | S. aureus Cas9 with PAM interaction domain from |
| cCas9 | | SaCas9 orthologues, expands recognition and |
| | | targetability of NNVRRN, NNVACT, NNVATG, |
| | | NNVATT, NNVGCT, NNVGTG, and NNVGTT PAM |
| | | sequences |
| Streptococcus | doi.org/10.1 | Recognizes 5'-NAA-3' PAM |
| macacae (Smac) | 101/429654 | |
| Cas9 NCTC 11558 | | |
| Spy-mac Cas9, | 32424114 | Recognizes 5'-NAA-3' PAM |
| Smac-py Cas9, | | |
| iSpyMac | | |
| N. meningitidis | 30581144 | Recognizes N4CC PAM |
| Nme2Cas9 | | |
| SpG Cas9 | 32217751; | SpCas9 variant capable of targeting NGN PAMs |
| (SEQ-ID 158) | WO | D1135L/S1136W/G1218K/E1219Q/R1335Q/T1337R |
| | 2021151085 | Also as SpG-HF1 in combination with |
| | | N497A/R661A/Q695A/Q926A |
| SpRY Cas9 | 32217751; | SpCas9 variant capable of targeting NRN>NYN PAMs |
| (SEQ-ID 157) | WO | SpRY(A61R/L1111R/D1135L/S1136W/G1218K/E121 |
| | 2021151085 | 9Q/N1317R/A1322R/R1333P/R1335Q/T1337R); also |
| | | as SpRY-HF1 in combination with |
| | | N497A/R661A/Q695A/Q926A |
| S. pyogenes Cas9 | 32042170 | D10T(optional)/1322V/S409I/E427G/R654L/R753G/R |
| (SpCas9) SpCas9- | | 1114G/D1135N/E1219V/D1332N/R1335Q/T1337N/S1 |
| NRCH | | 338T/H1349R |
| S. pyogenes Cas9 | 32042170 | D10T(optional)/1322V/S409I/E427G/R654L/R753G/R |
| (SpCas9) SpCas9- | | 1114G/D1135N/V1139A/D1180G/E1219V/Q1221H/A |
| NRRH | | 1320V/R1333K |
| S. pyogenes Cas9 | 32042170 | D10T(optional)/1322V/S409I/E427G/R654L/R753G/R |
| (SpCas9) SpCas9- | | 1114G/D1135N/D1180G/G1218S/E1219V/Q1221H/P1 |
| NRTH | | 249S/E1253K/P1321S/D1332G/R1335L |

*predicted based on UniRule annotation on the UniProt database.

TABLE E
List of Exemplary SpCas9 Activity-Altering Mutations

| | PMID or | |
|---|---|---|
| Name of enzyme | patent ID | Mutations |
| S. pyogenes Cas9 | 26098369, | L1111H, G1218R, T1337R |
| (SpCas9) | 26735016 | |
| SpCas9-VRQR | | |
| S. pyogenes Cas9 | PCT/ | S55R, A61R, R221K, G366R, N394K, |
| (SpCas9) | US2021/ | G1104K, G1104R, L1111R, T1138K, |
| (various | 014933 | T1138R, V1139T, V1139S, K115IR, |
| mutations) | | K1153R, K1200R, G1218R, E1243K, |
| | | L1245V, E1253K, A1285R, A1285K, |
| | | K1289R, T1314R, N1317R, G1219R, |
| | | G1319H, A1320S, A1320T, A1322S, |
| | | A1322T, A1322R, D1332R, D1332K, |
| | | D1332H, D1332Q, D1332N, |
| | | A61R/N1317R, G1104K/N1317R, |
| | | A61R/G1104K, S55R/G1104K, |
| | | S55R/A61R, SSSR/N1317R, |
| | | G1104K/T1314R, A61R/T1314R, |
| | | T1314R/N1317R, S55R/T1314R, |
| | | A61R/G1104R, G1104R/N1317R, |
| | | S55R/G1104R, G1104R/T1314R, |
| | | G366R/G1104K, S55R/G366R, |
| | | A61R/G366R, G366R/N1317R, |
| | | G366R/T1314R, G366R/G1104R. |
| | | G1104K/A128SR, A61R/A1285R, |
| | | A1285R/N1317R, S55R/A1285R, |
| | | A1285R/T1314R, G1104R/A1285R, |
| | | G366R/A1285R, G1104K/A1285K, |
| | | A61R/A128SK, A1285K/N1317R, |
| | | S55R/A1285K, A1285K/T1314R, |
| | | G1104R/A1285K, G366R/A1285K, |
| | | G1104K/D1332R, A61R/D1332R. |
| | | D1332R/N1317R, SSSR/D1332R, |
| | | D1332R/T1314R, G1104R/D1332R, |
| | | G366R/D1332R, A1285K/D1332R, |
| | | A1285R/D1332R, |
| | | A61R/G1104K/N1317R, A61R/L111IR, |
| | | A61R/A1322R, A61R/L1111R/A1322R, |
| | | G1104K/L111IR, G1104K/A1322R. |
| | | G1104K/L1111R/A1322R, |
| | | N1317R/L1111R, N1317R/A1322R, |
| | | N1317R/L1111R/A1322R, |
| | | A61R/N1317R/L1111R, |
| | | A61R/N1317R/A1322R, |
| | | A61R/N1317R/L111IR/A1322R, |
| | | G1104K/N1317R/L1111R, |
| | | G1104K/N1317R/A1322R, |
| | | G1104K/N1317R/L 1111R/A1322R, |
| | | A61R/G1104K/L 1111R. |
| | | A61R/G1104K/A1322R, |
| | | A61R/G1104K/L1111R/A1322R, |
| | | SSSR/L1111R, S55R/A1322R, |
| | | S55R/L1111R/A1322R, G366R/LI11IR, |
| | | G366R/A1322R, G366R/L |
| | | 1111R/A1322R, N394K/L111IR, |
| | | N394K/A1322R, |
| | | N394K/L1111R/A1322R, |
| | | A1285K/L1111R, A1285K/A1322R. |
| | | A1285K/L1111R/A1322R. |
| | | D1332K/L1111R, D1332K/A1322R, |
| | | D1332K/L1111R/A1322R, R691A, |
| | | M495V, Y515N, K526E, R661Q, |
| | | R661L, R661S, Y450A/Q695A, |
| | | L169A/Q695A, Q695A/Q926A, |
| | | Q695A/D1135E, Q926A/D1135E, |
| | | Y450A/D1135E, L169A/Y450A/Q695A, |
| | | L169A/Q695A/Q926A, |
| | | Y450A/Q695A/Q926A, |
| | | R661A/Q695A/Q926A, |
| | | N497A/Q695A/Q926A, |
| | | Y450A/Q695A/D1135E, |
| | | Y450A/Q926A/D 113SE, |
| | | Q695A/Q926A/D1135E, |
| | | L169A/Y450A/Q695A/Q926A, |
| | | L169A/R661A/Q695A/Q926A, |
| | | Y450A/R661A/Q695A/Q926A, |
| | | N497A/Q695 A/Q926A/D1135E, |
| | | R661A/Q695A/Q926A/D1135E, and |
| | | Y450A/Q695A/Q926A/D1135E; |
| | | N692A/M694A/Q695A/H698A, |
| | | N692A/M694A/Q695A/H698A/Q926A; |
| | | N692A/M694A/Q695A/Q926A; |
| | | N692A/M694A/H698A/Q926A; |
| | | N692A/Q695A/H698A/Q926A; |
| | | M694A/Q695A/H698A/Q926A; |
| | | N692A/Q695A/H698A; |
| | | N692A/M694A/Q695A; |
| | | N692A/H698A/Q926A; |
| | | N692A/M694A/Q926A; |
| | | N692A/M694A/H698A; |
| | | M694A/Q695A/H698A; |
| | | M694A/Q695A/Q926A; |
| | | Q695A/H698A/Q926A; |
| | | G582A/V583A/E584A/D585A/N588A/ |
| | | Q926A; |
| | | G582A/V583A/E584A/D585A/N588A; |
| | | T657A/G658A/W659A/R661A/Q926A; |
| | | T657A/G658A/W659A/R661A; |
| | | F491A/M495A/T496A/N497A/Q926A; |
| | | F491A/M495A/T496A/N497A; |
| | | K918A/V922A/R925A/Q926A; or |
| | | 918A/V922A/R925A; K855A; |
| | | K810A/K1003A/R1060A; |
| | | K848A/K1003A/R1060A: |
| | | M495V/Y515N/K526E/R661Q; |
| | | M495V/Y515N/K526E/R661L; or |
| | | M495V/Y515N/K526E/R661S |
| S. pyogenes Cas9 | 32217751 | A61R, G1104K (not present in final |
| (SpCas9) | | SpRY enzyme), L1111R, N1317R, |
| SpRY | | A1322R |
| S. pyogenes Cas9 | 30166441 | L1111R, A1322R |
| (SpCas9) | | |
| SpCas9-NG | | |
| S. pyogenes Cas9 | 29203891 | R221K, L244Q, N349K, E1243K, |
| (SpCas9) | | L1245V, E1253K |
| (various | | |
| mutations) | | |
| S. pyogenes Cas9 | 26990992 | N1286Q, I1331F, D1332K, |
| (SpCas9) | | |
| SpCas 9-QQR | | |
| S. pyogenes Cas9 | 25207793 | D147Y, P411T |
| (SpCas9) | | |
| iCas9 | | |
| S. pyogenes Cas9 | 36917274 | L1206P |
| (SpCas9) | | |
| SpCas9-L1206P | | |

### Effector Proteins

The methods and compositions described herein further include effector proteins that have DNA polymerase activity, e.g., DNA-dependent DNA polymerases, e.g., of family A, B, C, D, X, or Y, or RNA-dependent DNA polymerases (also known as reverse transcriptases) (for polymerase click editors (PCEs)). Exemplary polymerases include E. coli Klenow (EcKlenow, optionally with the D355A and/or E357A mutations that deactivate its 3′-5′ exonuclease domain); Taq Stoffel; Pol-Beta; Pol-Beta+Sso7d; Phi29 DNA Polymerase (D169A); Sequenase; T4 DNA Polymerase, and E. coli dKlenow (optionally with the D355A, E357A, D705A, and/or D882A mutations). Exemplary reverse transcriptases are described in more detail below. In some methods a ligase is used; exemplary DNA Ligases include T3 DNA ligase; T4 DNA ligase; T7 DNA ligase; ChlV (SplintR) DNA ligase; PhiKMV DNA ligase; Vaccinia DNA ligase; E. coli DNA ligase, Taq DNA ligase; 9° C. DNA ligase; or Hi-T4 DNA Ligase. Methods that use a clkDNA would need a DNA-dependent DNA polymerase, and methods that use a pegRNA would need an RNA-dependent DNA polymerase.

Previous literature on Taq DNA polymerase demonstrated activity-enhancing mutations at amino acid positions 732 (D732N)¹⁶, 507¹⁷, 543¹⁸, 605/617¹⁹, 685/686/687 (U.S. Ser. No. 11/046,939B2), and 742/743 with or without a basic residue insertion of length 3 or length 9 between positions 738 and 739²⁰. EcKlenow is also structurally homologous to TaqStoffel, hinting that analogous mutations may also increase the activity of EcKlenow. Moreover, non-specific DNA binding domains (e.g. villin headpiece, supercharged villin headpiece, Sso7d, NeqSSB, etc.) fused to the N- or C-terminus of DNA polymerases have been shown to increase the DNA affinity, stability, and processivity of the polymerase²¹⁻²³. Exchange of the 3′-5′ exonuclease domain of TaqStoffel with that of EcKlenow may also endow TaqStoffel with proofreading capability, as has been done previously²⁴. Thus, the present compositions and methods can use a DNA polymerase, such as EcKlenow or TaqStoffel, which include one or more of these modifications.

### Reverse Transcriptases (RTs), Reduced Size RTs, and Variant RTs

The present compositions and methods can use any RT, including Group II introns. Group II introns are retroelements that consist of a self-splicing ribozyme and an intron encoded protein (IEP) which functions as a reverse transcriptase (RT), DNA endonuclease, and RNA maturase.

In some embodiments, the pentamutant Moloney Murine Leukemia Virus reverse transcriptase (MMLV-RT) can be used. The group II intron RT (commercially available as “MarathonRT”) from Eubacterium rectale (E.r.) has been shown to display superior intrinsic RT processivity compared to Superscript IV. As shown herein, substitution of the M-MLV RT in a PE with MarathonRT or other RTs resulted in efficient prime editing in the HEK293T cell line. Additional Exemplary alternative RTs include those listed in Table F, below.

TABLE F
Alternative reverse transcriptases

| | NCBI or Uniprot | Reverse |
|---|---|---|
| Organism | Acc. No. or Source | Transcriptase Type |
| Geobacillus | E2GM63 (uniport) | Group Il Intron |
| stearothermophilus* | | |
| Lactococcus lactis | AAB06503.1 | Group Il Intron |
| subsp. lactis | | |
| Thermosynechococcus | BAC08171.1 | Group Il Intron |
| elongatus BP-1 | | |
| Sinorhizobium meliloti | WP_010967953.1 | Group Il Intron |
| Methanosarcina | AAM07961.1 | Group Il Intron |
| acetivorans C2A | | |
| Enterobacter cloacae | AEC33268.1 | Group II Intron |
| Clostridium | NP_350100.1 | Group II Intron |
| acetobutylicum ATCC | | |
| 824 | | |
| Bacillus halodurans | BAA90841.1 | Group Il Intron |
| Pseudomonas | AAB68949.1 | Group Il Intron |
| alcaligenes | | |
| Pseudomonas putida | CAB81565.1 | Group Il Intron |
| Streptococcus | CAC35989.1 | Group Il Intron |
| agalactiae | | |
| Roseburia intestinalis | D4L313 (uniprot) | Group II Intron |
| Eubacterium rectale | CBK92290.1 | Group Il Intron |
| (marathonRT) | | |
| Streptococcus | WP_013851921.1 | Group II Intron |
| pasteurianus | | |
| Shigella sonnei | WP_077124660.1 | Group Il Intron |
| Saccharomyces | NP_009310.1 | Group Il Intron |
| cerevisiae S288C | | (yeast) |
| (yeast) | | |
| Saccharomyces | NP_009309.1 | Group Il Intron |
| cerevisiae S288C | | (yeast) |
| (yeast) | | |
| Bordetella virus BPP1 | AAR97672.1 | Diversity Generating |
| | | Retroelement |
| ANMV-1 virus | AJP62064.1 | Diversity Generating |
| | | Retroelement |
| Bacteroides phage | DAC76693.1 | Diversity Generating |
| p00 | | Retroelement |
| Treponema denticola | AAS12785.1 | Diversity Generating |
| ATCC 35405 | | Retroelement |
| archaeon | AJF63168.1 | Diversity Generating |
| GW2011_AR20 | | Retroelement |
| Baboon endogenous | YP_009109694.1 | Retrovirus |
| virus strain M7 | | |
| Feline leukemia virus | NP_047255.1 | Retrovirus |
| Human foamy virus | CAA68999.1 | Retrovirus |
| Feline | AAB59937.1 | Retrovirus |
| immunodeficiency | | |
| virus | | |
| Human Endogenous | Nam Lee, et. al | Retrovirus |
| Retrovirus K | (2007) | |
| (reconstituted) | | |
| Necator americanus | XP_013295720.1 | Group Il intron |
| | | (eukaryotic) |
| Axinella verrucosa | CRX66588.1 | Group Il intron |
| | | (eukaryotic) |
| Axinella verrucosa | CRX66589.1 | Group Il intron |
| | | (eukaryotic) |
| Xenopolymerase | Jared W. Ellefson, | Thermococcus |
| RTX | et. al (2016) | kodakarensis |
| | | (engineered) |

*Geobacillus stearothermophilus GsI-IIC intron RT (denoted GsI-IIC RT; sold commercially as TGIRT-III; InGex); see Stamos et al., Mol Cell. 2017 December 7; 68(5): 926-939.e4.

Exemplary RT sequences include:

Eubacterium rectale RT (aka Marathon-RT; WT)
SEQ ID NO: 63
MDTSNLMEQILSSDNLNRAYLQVVRNKGAEGVDGMKYTELKEHL
AKNGETIKGQLRTRKYKPQPARRVEIPKPDGGVRNLGVPTVTDR
FIQQAIAQVLTPIYEEQFHDHSYGFRPNRCAQQAILTALNIMND
GNDWIVDIDLEKFFDTVNHDKLMTLIGRTIKDGDVISIVRKYLV
SGIMIDDEYEDSIVGTPQGGNLSPLLANIMLNELDKEMEKRGLN
FVRYADDCIIMVGSEMSANRVMRNISRFIEEKLGLKVNMTKSKV
DRPSGLKYLGFGFYFDPRAHQFKAKPHAKSVAKFKKRMKELTCR
SWGVSNSYKVEKLNQLIRGWINYFKIGSMKTLCKELDSRIRYRL
RMCIWKQWKTPQNQEKNLVKLGIDRNTARRVAYTGKRIAYVCNK
GAVNVAISNKRLASFGLISMLDYYIEKCVTC
Human endogenous retrovirus K consensus
(HERV-Kcon) RT
SEQ ID NO: 64
MKSRKRRNRVSFLGAATVEPPKPIPLTWKTEKPVWVNQWPLPKQ
KLEALHLLANEQLEKGHIEPSFSPWNSPVFVIQKKSGKWRMLTD
LRAVNAVIQPMGPLQPGLPSPAMIPKDWPLIIIDLKDCFFTIPL
AEQDCEKFAFTIPAINNKEPATRFQWKVLPQGMLNSPTICQTFV
GRALQPVREKFSDCYIIHYIDDILCAAETKDKLIDCYTFLQAEV
ANAGLAIASDKIQTSTPFHYLGMQIENRKIKPQKIEIRKDTLKT
LNDFQKLLGDINWIRPTLGIPTYAMSNLFSILRGDSDLNSKRML
TPEATKEIKLVEEKIQSAQINRIDPLAPLQLLIFATAHSPTGII
IQNTDLVEWSFLPHSTVKTFTLYLDQIATLIGQTRLRIIKLCGN
DPDKIVVPLTKEQVRQAFINSGAWQIGLANFVGIIDNHYPKTKI
FQFLKLTTWILPKITRREPLENALTVFTDGSSNGKAAYTGPKER
VIKTPYQSAQRAELVAVITVLQDFDQPINIISDSAYVVQATRDV
ETALIKYSMDDQLNQLFNLLQQTVRKRNFPFYITHIRAHTNLPG
PLTKANEQADLLVSSALIKAQELHA
Geobacillus stearothermophilus GsI-IIC RT
(WT)
SEQ ID NO: 65
MALLERILARDNLITALKRVEANQGAPGIDGVSTDQLRDYIRAH
WSTIHAQLLAGTYRPAPVRRVEIPKPGGGTRQLGIPTVVDRLIQ
QAILQELTPIFDPDFSSSSFGFRPGRNAHDAVRQAQGYIQEGYR
YVVDMDLEKFFDRVNHDILMSRVARKVKDKRVLKLIRAYLQAGV
MIEGVKVQTEEGTPQGGPLSPLLANILLDDLDKELEKRGLKFCR
YADDCNIYVKSLRAGQRVKQSIQRFLEKTLKLKVNEEKSAVDRP
WKRAFLGFSFTPERKARIRLAPRSIQRLKQRIRQLTNPNWSISM
PERIHRVNQYVMGWIGYFRLVETPSVLQTIEGWIRRRLRLCQWL
QWKRVRTRIRELRALGLKETAVMEIANTRKGAWRTTKTPQLHQA
LGKTYWTAQGLKSLTQRYFELRQG

Geobacillus stearothermophilus GsI-IIC intron RT (GsI-IIC RT) pentamutants can also be used, e.g., comprising mutations D11R/N23R/G71R/G113K/P194R (positions bolded in SEQ ID NO:xxx, above.

Exemplary MMLV RT sequences include the following:

MMLV-RT pentamutant (used in classic PE2),
without NLS, starts with T (not M)
SEQ ID NO: 66
TLNIEDEYRLHETSKEPDVSLGSTWLSDFPQAWAETGGMGLAVR
QAPLIIPLKATSTPVSIKQYPMSQEARLGIKPHIQRLLDQGILV
PCQSPWNTPLLPVKKPGTNDYRPVQDLREVNKRVEDIHPTVPNP
YNLLSGLPPSHQWYTVLDLKDAFFCLRLHPTSQPLFAFEWRDPE
MGISGQLTWTRLPQGFKNSPTLFNEALHRDLADFRIQHPDLILL
QYVDDLLLAATSELDCQQGTRALLQTLGNLGYRASAKKAQICQK
QVKYLGYLLKEGQRWLTEARKETVMGQPTPKTPRQLREFLGKAG
FCRLFIPGFAEMAAPLYPLTKPGTLFNWGPDQQKAYQEIKQALL
TAPALGLPDLTKPFELFVDEKQGYAKGVLTQKLGPWRRPVAYLS
KKLDPVAAGWPPCLRMVAAIAVLTKDAGKLTMGQPLVILAPHAV
EALVKQPPDRWLSNARMTHYQALLLDTDRVQFGPVVALNPATLL
PLPEEGLQHNCLDILAEAHGTRPDLTDQPLPDADHTWYTDGSSL
LQEGQRKAGAAVTTETEVIWAKALPAGTSAQRAELIALTQALKM
AEGKKLNVYTDSRYAFATAHIHGEIYRRRGWLTSEGKEIKNKDE
ILALLKALFLPKRLSIIHCPGHQKGHSAEARGNRMADQAARKAA
ITETPDTSTLLIENSSP

The present compositions and methods can make use of variants as known in the art and as provided herein, e.g., MarathonRT, GsI-IIC RT, and MMLV-RT variants, e.g., PE2 MMLV RT (with D200N, T306K, W313F, T330P, L603W mutations), or MMLV or PE2 MMLV RT truncations (truncations 2, 5, and 6; Grunewald et al. Nat Biotechnol. 2023 March; 41(3):337-343), as well as RT HFV, HERV, LtrA, HERV-Kcon, Tel4c, Marathon, GsI-IIC, Ma-Int5, engineered Marathon (optionally with D14R, N26R, D74R, N116K, or N197R mutations), etc.

### Recombinase

Additional effectors (such as serine recombinases) can be included in the present proteins and compositions. For example, The PCEs, LCEs, or dual-overhang ligation approaches can be used to install a recombinase attachment site (att) at a desired position in the genome. Serine recombinases, either fused to a PCE/LCE or expressed in trans, integrate a DNA donor containing a corresponding recombinase attachment site and a cargo of interest at the targeted location. Serine recombinases can include BxbI, Pa01, BceINT, etc (including those discovered from metagenomic mining efforts as described in Ref²⁵).

clkDNA Templates

The clkDNA templates used in the present compositions and methods include (i) a localization moiety, (ii) a polymerization template (PT), and (iii) a flap binding region (FBR). In some embodiments, the clkDNA templates are in the order (i)-(ii)-(iii) from 5′ to 3′, but other configurations are possible (e.g. (ii)-(iii)-(i), e.g., wherein the clkDNA has a 3′ moiety (e.g., chloroalkane, etc combined with a SNAP tag) rather than an HUH).

The localization moiety is a sequence or modification that binds or links to the tethering domain on the DBDn, e.g., an HUH endonuclease recognition site (when the CE includes an HUH), biotin (when the CE includes avidin), label with O⁶-benzylguanine derivatives (when the CE includes SNAP), label with O²-benzylcytosine derivatives (when the CE includes CLIP-tag), and labeled with a chloroalkane (when the CE includes HALO-tag). RNA or DNA hairpins can also be used to localize effectors (when the CE includes an RNA or DNA binding protein, such as a phage coat protein like MCP, PCP, BoxB, or Com).

The polymerization template (PT) for use with PCEs includes a portion that encodes homology to the target genome, e.g., at least 3, 4, 5, 6, 7, 8, 9, or 10 nt long, and optionally up to 50, 100, 200, 250, or 500 nt long, and a portion that includes the edit that is at least 1 nt long.

The flap binding region is complementary or partly complementary to the genomic flap released by the nickase (and thus in some embodiments the FBR is complementary or partly complementary to part of the gRNA protospacer sequence). In general, the length of the genomic flap is the distance between the DNA nick on the NTS and equivalent NTS position that is analogous to the end of the TS/gRNA spacer, which will often be about 15-20, e.g., 17, nt but it can be target specific. In some embodiments, the flap can be shorter (e.g., in the case of truncated gRNAs (Fu et al., Nat Biotechnol. 2014 March; 32(3): 279-284) if the gRNA spacer region is shorter. In some embodiments, the flap can be longer, though such arrangements may be thermodynamically less favorable, if the TS/NTS is unpaired outside of the gRNA spacer/TS region.

In some methods, more than two clkDNAs and gRNAs can be used when doing multiplex labeling of multiple fragments (either single or dual end). The labeling can be done all in a single reaction, with different ACOs/adapters depending on the fragment being labeled. Each ACO will have a corresponding adapter sequence.

### EXEMPLARY SEQUENCES AND CONSTRUCTS

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm that has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Amino acid sequence of an exemplary protein
expression construct for Sumo-PCV2-gsXTENgs-
SpCas9-NLS-6xHis (SEQ ID NO: 93)
(SEQ ID NO: 67)
MSDSEVNQEAKPEVKPEVKPETHINLKVSDGSSEIFFKIKKTTP
LRRLMEAFAKRQGKEMDSLRFLYDGIRIQADQTPEDLDMEDNDI
IEAHREQIGGSPSKKNGRSGPQPHKRWVFTLNNPSEDERKKIRD
LPISLFDYFIVGEEGNEEGRTPHLQGFANFVKKQTFNKVKWYLG
ARCHIEKAKGTDQQNKEYCSKEGNLLMECGAPRSQGQRSGGSSG
SETPGTSESATPESSGGSDKKYSIGLDIGTNSVGWAVITDEYKV
PSKKFKVLGNTDRHSIKKNLIGALLFDSGETAEATRLKRTARRR
YTRRKNRICYLQEIFSNEMAKVDDSFFHRLEESFLVEEDKKHER
HPIFGNIVDEVAYHEKYPTIYHLRKKLVDSTDKADLRLIYLALA
HMIKFRGHFLIEGDLNPDNSDVDKLFIQLVQTYNQLFEENPINA
SGVDAKAILSARLSKSRRLENLIAQLPGEKKNGLFGNLIALSLG
LTPNFKSNFDLAEDAKLQLSKDTYDDDLDNLLAQIGDQYADLFL
AAKNLSDAILLSDILRVNTEITKAPLSASMIKRYDEHHQDLTLL
KALVRQQLPEKYKEIFFDQSKNGYAGYIDGGASQEEFYKFIKPI
LEKMDGTEELLVKLNREDLLRKQRTFDNGSIPHQIHLGELHAIL
RRQEDFYPFLKDNREKIEKILTFRIPYYVGPLARGNSRFAWMTR
KSEETITPWNFEEVVDKGASAQSFIERMTNFDKNLPNEKVLPKH
SLLYEYFTVYNELTKVKYVTEGMRKPAFLSGEQKKAIVDLLFKT
NRKVTVKQLKEDYFKKIECFDSVEISGVEDRFNASLGTYHDLLK
IIKDKDFLDNEENEDILEDIVLTLTLFEDREMIEERLKTYAHLF
DDKVMKQLKRRRYTGWGRLSRKLINGIRDKQSGKTILDFLKSDG
FANRNFMQLIHDDSLTFKEDIQKAQVSGQGDSLHEHIANLAGSP
AIKKGILQTVKVVDELVKVMGRHKPENIVIEMARENQTTQKGQK
NSRERMKRIEEGIKELGSQILKEHPVENTQLQNEKLYLYYLQNG
RDMYVDQELDINRLSDYDVDHIVPQSFLKDDSIDNKVLTRSDKN
RGKSDNVPSEEVVKKMKNYWRQLLNAKLITQRKFDNLTKAERGG
LSELDKAGFIKRQLVETRQITKHVAQILDSRMNTKYDENDKLIR
EVKVITLKSKLVSDFRKDFQFYKVREINNYHHAHDAYLNAVVGT
ALIKKYPKLESEFVYGDYKVYDVRKMIAKSEQEIGKATAKYFFY
SNIMNFFKTEITLANGEIRKRPLIETNGETGEIVWDKGRDFATV
RKVLSMPQVNIVKKTEVQTGGFSKESILPKRNSDKLIARKKDWD
PKKYGGFDSPTVAYSVLVVAKVEKGKSKKLKSVKELLGITIMER
SSFEKNPIDFLEAKGYKEVKKDLIIKLPKYSLFELENGRKRMLA
SAGELQKGNELALPSKYVNFLYLASHYEKLKGSPEDNEQKQLFV
EQHKHYLDEIIEQISEFSKRVILADANLDKVLSAYNKHRDKPIR
EQAENIIHLFTLTNLGAPAAFKYFDTTIDRKRYTSTKEVLDATL
IHQSITGLYETRIDLSQLGGDGGGSGTRLPKKKRKVGGGSHHHH
HH
Amino acid sequence of an exemplary
tethering domain fused to a DNA-targeting
nuclease for CAGE using a PCV2-gsXTENgs-
SpCas9 fusion protein
(SEQ ID NO: 68)
MSPSKKNGRSGPQPHKRWVFTLNNPSEDERKKIRDLPISLFDYF
IVGEEGNEEGRTPHLQGFANFVKKQTFNKVKWYLGARCHIEKAK
GTDQQNKEYCSKEGNLLMECGAPRSQGQRSGGSSGSETPGTSES
ATPESSGGSDKKYSIGLDIGTNSVGWAVITDEYKVPSKKFKVLG
NTDRHSIKKNLIGALLFDSGETAEATRLKRTARRRYTRRKNRIC
YLQEIFSNEMAKVDDSFFHRLEESFLVEEDKKHERHPIFGNIVD
EVAYHEKYPTIYHLRKKLVDSTDKADLRLIYLALAHMIKFRGHF
LIEGDLNPDNSDVDKLFIQLVQTYNQLFEENPINASGVDAKAIL
SARLSKSRRLENLIAQLPGEKKNGLFGNLIALSLGLTPNFKSNF
DLAEDAKLQLSKDTYDDDLDNLLAQIGDQYADLFLAAKNLSDAI
LLSDILRVNTEITKAPLSASMIKRYDEHHQDLTLLKALVRQQLP
EKYKEIFFDQSKNGYAGYIDGGASQEEFYKFIKPILEKMDGTEE
LLVKLNREDLLRKQRTFDNGSIPHQIHLGELHAILRRQEDFYPF
LKDNREKIEKILTFRIPYYVGPLARGNSRFAWMTRKSEETITPW
NFEEVVDKGASAQSFIERMTNFDKNLPNEKVLPKHSLLYEYFTV
YNELTKVKYVTEGMRKPAFLSGEQKKAIVDLLFKTNRKVTVKQL
KEDYFKKIECFDSVEISGVEDRFNASLGTYHDLLKIIKDKDFLD
NEENEDILEDIVLTLTLFEDREMIEERLKTYAHLFDDKVMKQLK
RRRYTGWGRLSRKLINGIRDKQSGKTILDFLKSDGFANRNFMQL
IHDDSLTFKEDIQKAQVSGQGDSLHEHIANLAGSPAIKKGILQT
VKVVDELVKVMGRHKPENIVIEMARENQTTQKGQKNSRERMKRI
EEGIKELGSQILKEHPVENTQLQNEKLYLYYLQNGRDMYVDQEL
DINRLSDYDVDHIVPQSFLKDDSIDNKVLTRSDKNRGKSDNVPS
EEVVKKMKNYWRQLLNAKLITQRKFDNLTKAERGGLSELDKAGF
IKRQLVETRQITKHVAQILDSRMNTKYDENDKLIREVKVITLKS
KLVSDFRKDFQFYKVREINNYHHAHDAYLNAVVGTALIKKYPKL
ESEFVYGDYKVYDVRKMIAKSEQEIGKATAKYFFYSNIMNFFKT
EITLANGEIRKRPLIETNGETGEIVWDKGRDFATVRKVLSMPQV
NIVKKTEVQTGGFSKESILPKRNSDKLIARKKDWDPKKYGGFDS
PTVAYSVLVVAKVEKGKSKKLKSVKELLGITIMERSSFEKNPID
FLEAKGYKEVKKDLIIKLPKYSLFELENGRKRMLASAGELQKGN
ELALPSKYVNFLYLASHYEKLKGSPEDNEQKQLFVEQHKHYLDE
IIEQISEFSKRVILADANLDKVLSAYNKHRDKPIREQAENIIHL
FTLTNLGAPAAFKYFDTTIDRKRYTSTKEVLDATLIHQSITGLY
ETRIDLSQLGGD
Amino acid sequence of an exemplary DNA-
dependent polymerase for CAGE or CAPTURE
using E. coli Klenow fragment
(SEQ ID NO: 69)
VISYDNYVTILDEETLKAWIAKLEKAPVFAFATATDSLDNISAN
LVGLSFAIEPGVAAYIPVAHDYLDAPDQISRERALELLKPLLED
EKALKVGQNLKYDRGILANYGIELRGIAFDTMLESYILNSVAGR
HDMDSLAERWLKHKTITFEEIAGKGKNQLTFNQIALEEAGRYAA
EDADVTLQLHLKMWPDLQKHKGPLNVFENIEMPLVPVLSRIERN
GVKIDPKVLHNHSEELTLRLAELEKKAHEIAGEEFNLSSTKQLQ
TILFEKQGIKPLKKTPGGAPSTSEEVLEELALDYPLPKVILEYR
GLAKLKSTYTDKLPLMINPKTGRVHTSYHQAVTATGRLSSTDPN
LQNIPVRNEEGRRIRQAFIAPEDYVIVSADYSQIELRIMAHLSR
DKGLLTAFAEGKDIHRATAAEVFGLPLETVTSEQRRSAKAINFG
LIYGMSAFGLARQLNIPRKEAQKYMDLYFERYPGVLEYMERTRA
QAKEQGYVETLDGRRLYLPDIKSSNGARRAAAERAAINAPMQGT
AADIIKRAMIAVDAWLQAEQPRVRMIMQVHDELVFEVHKDDVDA
VAKQIHQLMENCTRLDVPLLVEVGSGENWDQAH

Also available commercially as DNA Polymerase I, Large (Klenow) Fragment from New England Biolabs (Catalog #M0210)

Amino acid sequence of Streptococcus
pyogenes Cas9 (SpCas9) as an exemplary
nuclease protein for CAGE or PE-CAGE
(SEQ ID NO: 70)
MDKKYSIGLDIGTNSVGWAVITDEYKVPSKKFKVLGNTDRHSIK
KNLIGALLFDSGETAEATRLKRTARRRYTRRKNRICYLQEIFSN
EMAKVDDSFFHRLEESFLVEEDKKHERHPIFGNIVDEVAYHEKY
PTIYHLRKKLVDSTDKADLRLIYLALAHMIKFRGHFLIEGDLNP
DNSDVDKLFIQLVQTYNQLFEENPINASGVDAKAILSARLSKSR
RLENLIAQLPGEKKNGLFGNLIALSLGLTPNFKSNFDLAEDAKL
QLSKDTYDDDLDNLLAQIGDQYADLFLAAKNLSDAILLSDILRV
NTEITKAPLSASMIKRYDEHHQDLTLLKALVRQQLPEKYKEIFF
DQSKNGYAGYIDGGASQEEFYKFIKPILEKMDGTEELLVKLNRE
DLLRKQRTFDNGSIPHQIHLGELHAILRRQEDFYPFLKDNREKI
EKILTFRIPYYVGPLARGNSRFAWMTRKSEETITPWNFEEVVDK
GASAQSFIERMTNFDKNLPNEKVLPKHSLLYEYFTVYNELTKVK
YVTEGMRKPAFLSGEQKKAIVDLLFKTNRKVTVKQLKEDYFKKI
ECFDSVEISGVEDRFNASLGTYHDLLKIIKDKDFLDNEENEDIL
EDIVLTLTLFEDREMIEERLKTYAHLFDDKVMKQLKRRRYTGWG
RLSRKLINGIRDKQSGKTILDFLKSDGFANRNFMQLIHDDSLTF
KEDIQKAQVSGQGDSLHEHIANLAGSPAIKKGILQTVKVVDELV
KVMGRHKPENIVIEMARENQTTQKGQKNSRERMKRIEEGIKELG
SQILKEHPVENTQLQNEKLYLYYLQNGRDMYVDQELDINRLSDY
DVDHIVPQSFLKDDSIDNKVLTRSDKNRGKSDNVPSEEVVKKMK
NYWRQLLNAKLITQRKFDNLTKAERGGLSELDKAGFIKRQLVET
RQITKHVAQILDSRMNTKYDENDKLIREVKVITLKSKLVSDFRK
DFQFYKVREINNYHHAHDAYLNAVVGTALIKKYPKLESEFVYGD
YKVYDVRKMIAKSEQEIGKATAKYFFYSNIMNFFKTEITLANGE
IRKRPLIETNGETGEIVWDKGRDFATVRKVLSMPQVNIVKKTEV
QTGGFSKESILPKRNSDKLIARKKDWDPKKYGGFDSPTVAYSVL
VVAKVEKGKSKKLKSVKELLGITIMERSSFEKNPIDFLEAKGYK
EVKKDLIIKLPKYSLFELENGRKRMLASAGELQKGNELALPSKY
VNFLYLASHYEKLKGSPEDNEQKQLFVEQHKHYLDEIIEQISEF
SKRVILADANLDKVLSAYNKHRDKPIREQAENIIHLFTLTNLGA
PAAFKYFDTTIDRKRYTSTKEVLDATLIHQSITGLYETRIDLSQ
LGGD
Amino acid sequence of an exemplary RNA-
dependent polymerase for PE-CAGE or PE-
CAPTURE using an engineered M-MLV RT
(bearing D200N, L603W, T306K, W313F, T330P
mutations)
(SEQ ID NO: 71)
TLNIEDEYRLHETSKEPDVSLGSTWLSDFPQAWAETGGMGLAVR
QAPLIIPLKATSTPVSIKQYPMSQEARLGIKPHIQRLLDQGILV
PCQSPWNTPLLPVKKPGTNDYRPVQDLREVNKRVEDIHPTVPNP
YNLLSGLPPSHQWYTVLDLKDAFFCLRLHPTSQPLFAFEWRDPE
MGISGQLTWTRLPQGFKNSPTLFNEALHRDLADFRIQHPDLILL
QYVDDLLLAATSELDCQQGTRALLQTLGNLGYRASAKKAQICQK
QVKYLGYLLKEGQRWLTEARKETVMGQPTPKTPRQLREFLGKAG
FCRLFIPGFAEMAAPLYPLTKPGTLFNWGPDQQKAYQEIKQALL
TAPALGLPDLTKPFELFVDEKQGYAKGVLTQKLGPWRRPVAYLS
KKLDPVAAGWPPCLRMVAAIAVLTKDAGKLTMGQPLVILAPHAV
EALVKQPPDRWLSNARMTHYQALLLDTDRVQFGPVVALNPATLL
PLPEEGLQHNCLDILAEAHGTRPDLTDQPLPDADHTWYTDGSSL
LQEGQRKAGAAVTTETEVIWAKALPAGTSAQRAELIALTQALKM
AEGKKLNVYTDSRYAFATAHIHGEIYRRRGWLTSEGKEIKNKDE
ILALLKALFLPKRLSIIHCPGHQKGHSAEARGNRMADQAARKAA
ITETPDTSTLLIENSSPSGGSKRTADGSEFE;

Also available commercially as SuperScript III Reverse Transcriptase from ThermoFisher Scientific (Catalog #18080044)

Amino acid sequence of an exemplary
tethering domain fused to an RNA-targeting
nuclease for CAPTURE using a PCV2-gsXTENgs-
Cas13 fusion protein, using Listeria
seeligeri serovar 1/2b Cas13 (LseCas13)
(SEQ ID NO: 72)
MSPSKKNGRSGPQPHKRWVFTLNNPSEDERKKIRDLPISLFDYF
IVGEEGNEEGRTPHLQGFANFVKKQTFNKVKWYLGARCHIEKAK
GTDQQNKEYCSKEGNLLMECGAPRSQGQRSGGSSGSETPGTSES
ATPESSGGSMWISIKTLIHHLGVLFFCDYMYNRREKKIIEVKTM
RITKVEVDRKKVLISRDKNGGKLVYENEMQDNTEQIMHHKKSSF
YKSVVNKTICRPEQKQMKKLVHGLLQENSQEKIKVSDVTKLNIS
NFLNHRFKKSLYYFPENSPDKSEEYRIEINLSQLLEDSLKKQQG
TFICWESFSKDMELYINWAENYISSKTKLIKKSIRNNRIQSTES
RSGQLMDRYMKDILNKNKPFDIQSVSEKYQLEKLTSALKATFKE
AKKNDKEINYKLKSTLQNHERQIIEELKENSELNQFNIEIRKHL
ETYFPIKKTNRKVGDIRNLEIGEIQKIVNHRLKNKIVQRILQEG
KLASYEIESTVNSNSLQKIKIEEAFALKFINACLFASNNLRNMV
YPVCKKDILMIGEFKNSFKEIKHKKFIRQWSQFFSQEITVDDIE
LASWGLRGAIAPIRNEIIHLKKHSWKKFFNNPTFKVKKSKIING
KTKDVTSEFLYKETLFKDYFYSELDSVPELIINKMESSKILDYY
SSDQLNQVFTIPNFELSLLTSAVPFAPSFKRVYLKGFDYQNQDE
AQPDYNLKLNIYNEKAFNSEAFQAQYSLFKMVYYQVFLPQFTTN
NDLFKSSVDFILTLNKERKGYAKAFQDIRKMNKDEKPSEYMSYI
QSQLMLYQKKQEEKEKINHFEKFINQVFIKGFNSFIEKNRLTYI
CHPTKNTVPENDNIEIPFHTDMDDSNIAFWLMCKLLDAKQLSEL
RNEMIKFSCSLQSTEEISTFTKAREVIGLALLNGEKGCNDWKEL
FDDKEAWKKNMSLYVSEELLQSLPYTQEDGQTPVINRSIDLVKK
YGTETILEKLFSSSDDYKVSAKDIAKLHEYDVTEKIAQQESLHK
QWIEKPGLARDSAWTKKYQNVINDISNYQWAKTKVELTQVRHLH
QLTIDLLSRLAGYMSIADRDFQFSSNYILERENSEYRVTSWILL
SENKNKNKYNDYELYNLKNASIKVSSKNDPQLKVDLKQLRLTLE
YLELFDNRLKEKRNNISHFNYLNGQLGNSILELFDDARDVLSYD
RKLKNAVSKSLKEILSSHGMEVTFKPLYQTNHHLKIDKLQPKKI
HHLGEKSTVSSNQVSNEYCQLVRTLLTMK
Amino acid sequence of an exemplary
polymerase click editor (PCE) comprising
PCV2-gsXTENgs-nSpCas9(H840A)-gsNLSgs-
EcKlenow
(SEQ ID NO: 73)
MSPSKKNGRSGPQPHKRWVFTLNNPSEDERKKIRDLPISLFDYF
IVGEEGNEEGRTPHLQGFANFVKKQTFNKVKWYLGARCHIEKAK
GTDQQNKEYCSKEGNLLMECGAPRSQGQRSGGSSGSETPGTSES
ATPESSGGSDKKYSIGLDIGTNSVGWAVITDEYKVPSKKFKVLG
NTDRHSIKKNLIGALLFDSGETAEATRLKRTARRRYTRRKNRIC
YLQEIFSNEMAKVDDSFFHRLEESFLVEEDKKHERHPIFGNIVD
EVAYHEKYPTIYHLRKKLVDSTDKADLRLIYLALAHMIKFRGHF
LIEGDLNPDNSDVDKLFIQLVQTYNQLFEENPINASGVDAKAIL
SARLSKSRRLENLIAQLPGEKKNGLFGNLIALSLGLTPNFKSNF
DLAEDAKLQLSKDTYDDDLDNLLAQIGDQYADLFLAAKNLSDAI
LLSDILRVNTEITKAPLSASMIKRYDEHHQDLTLLKALVRQQLP
EKYKEIFFDQSKNGYAGYIDGGASQEEFYKFIKPILEKMDGTEE
LLVKLNREDLLRKQRTFDNGSIPHQIHLGELHAILRRQEDFYPF
LKDNREKIEKILTFRIPYYVGPLARGNSRFAWMTRKSEETITPW
NFEEVVDKGASAQSFIERMTNFDKNLPNEKVLPKHSLLYEYFTV
YNELTKVKYVTEGMRKPAFLSGEQKKAIVDLLFKTNRKVTVKQL
KEDYFKKIECFDSVEISGVEDRFNASLGTYHDLLKIIKDKDFLD
NEENEDILEDIVLTLTLFEDREMIEERLKTYAHLFDDKVMKQLK
RRRYTGWGRLSRKLINGIRDKQSGKTILDFLKSDGFANRNFMQL
IHDDSLTFKEDIQKAQVSGQGDSLHEHIANLAGSPAIKKGILQT
VKVVDELVKVMGRHKPENIVIEMARENQTTQKGQKNSRERMKRI
EEGIKELGSQILKEHPVENTQLQNEKLYLYYLQNGRDMYVDQEL
DINRLSDYDVDAIVPQSFLKDDSIDNKVLTRSDKNRGKSDNVPS
EEVVKKMKNYWRQLLNAKLITQRKFDNLTKAERGGLSELDKAGF
IKRQLVETRQITKHVAQILDSRMNTKYDENDKLIREVKVITLKS
KLVSDFRKDFQFYKVREINNYHHAHDAYLNAVVGTALIKKYPKL
ESEFVYGDYKVYDVRKMIAKSEQEIGKATAKYFFYSNIMNFFKT
EITLANGEIRKRPLIETNGETGEIVWDKGRDFATVRKVLSMPQV
NIVKKTEVQTGGFSKESILPKRNSDKLIARKKDWDPKKYGGFDS
PTVAYSVLVVAKVEKGKSKKLKSVKELLGITIMERSSFEKNPID
FLEAKGYKEVKKDLIIKLPKYSLFELENGRKRMLASAGELQKGN
ELALPSKYVNFLYLASHYEKLKGSPEDNEQKQLFVEQHKHYLDE
IIEQISEFSKRVILADANLDKVLSAYNKHRDKPIREQAENIIHL
FTLTNLGAPAAFKYFDTTIDRKRYTSTKEVLDATLIHQSITGLY
ETRIDLSQLGGDSGGSSGGSKRTADGSEFESPKKKRKVSGGSSG
GSVISYDNYVTILDEETLKAWIAKLEKAPVFAFATATDSLDNIS
ANLVGLSFAIEPGVAAYIPVAHDYLDAPDQISRERALELLKPLL
EDEKALKVGQNLKYDRGILANYGIELRGIAFDTMLESYILNSVA
GRHDMDSLAERWLKHKTITFEEIAGKGKNQLTFNQIALEEAGRY
AAEDADVTLQLHLKMWPDLQKHKGPLNVFENIEMPLVPVLSRIE
RNGVKIDPKVLHNHSEELTLRLAELEKKAHEIAGEEFNLSSTKQ
LQTILFEKQGIKPLKKTPGGAPSTSEEVLEELALDYPLPKVILE
YRGLAKLKSTYTDKLPLMINPKTGRVHTSYHQAVTATGRLSSTD
PNLQNIPVRNEEGRRIRQAFIAPEDYVIVSADYSQIELRIMAHL
SRDKGLLTAFAEGKDIHRATAAEVFGLPLETVTSEQRRSAKAIN
FGLIYGMSAFGLARQLNIPRKEAQKYMDLYFERYPGVLEYMERT
RAQAKEQGYVETLDGRRLYLPDIKSSNGARRAAAERAAINAPMQ
GTAADIIKRAMIAVDAWLQAEQPRVRMIMQVHDELVFEVHKDDV
DAVAKQIHQLMENCTRLDVPLLVEVGSGENWDQAH

### EXAMPLES

The invention is further described in the following examples, which do not limit the scope of the invention described in the claims.

### Methods

The following materials and methods were used in the Examples below.

### Plasmids and Oligonucleotides

The protein expression plasmid for PCV2-SpCas9 was modified via isothermal assembly from Addgene plasmid #123643 to exchange the linker sequence between PCV2 and SpCas9 for a 24 amino acid gsXTENgs linker comprising the following sequence: SGGSSGSETPGTSESATPESSGGS (SEQ ID NO:74). clkDNAs, synthetic sgRNAs (Synthego), adaptor sequences, primers, and probes are listed in Table 1.

### PCV2-Cas9 Purification

PCV2-Cas9 was overexpressed in BL21(DE3) cells by IPTG induction. Protein was purified in an adapted protocol from Anders and Jinek⁸. Cells were pelleted and lysed in 50 mM HEPES, 200 mM NaCl, 20% (w/v) sucrose, 15 mM imidazole, pH 7.4, and eluted from a 5 ml EconoFit Ni-charged IMAC column (Bio-Rad) using a gradient from 15-500 mM imidazole followed by overnight incubation with SUMO protease Ulp1 at 4° C., while dialyzing into 50 mM HEPES, 150 mM KCl, 5% glycerol, 1 mM DTT, 1 mM EDTA, pH 7.5. Cation exchange was performed using a 5 ml HiTrap SP HP column (GE Healthcare), eluting using a gradient from 100 mM to 1 M KCl. Protein was concentrated in a 100 kDa MWCO spin concentrator (Amicon), frozen in liquid nitrogen and stored at −80° C. For experiments, a 60 μM of PCV2-Cas9 in 50% glycerol was used and stored at −20° C.

CAGE Reaction with Target PCR Substrate

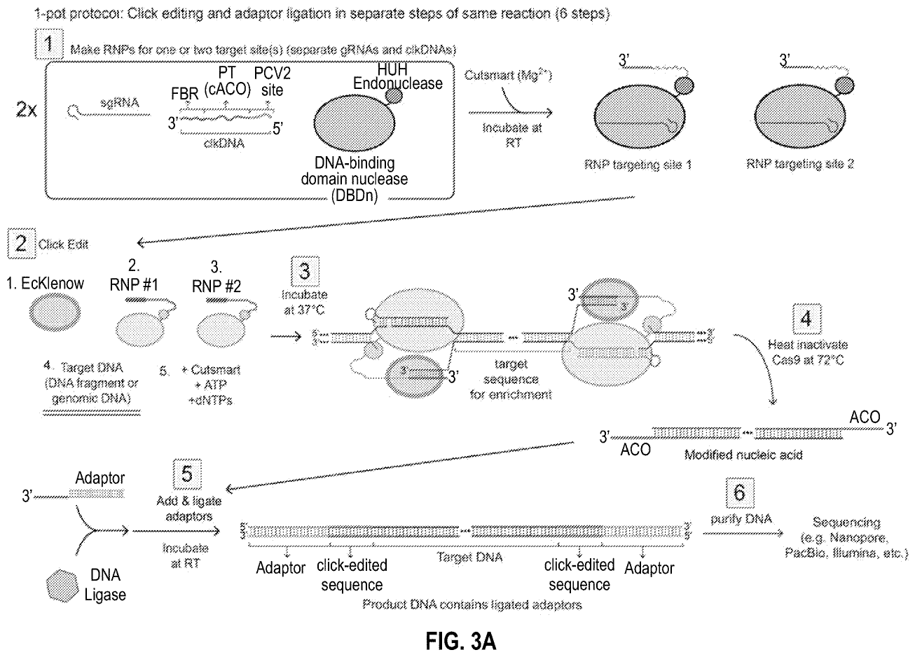

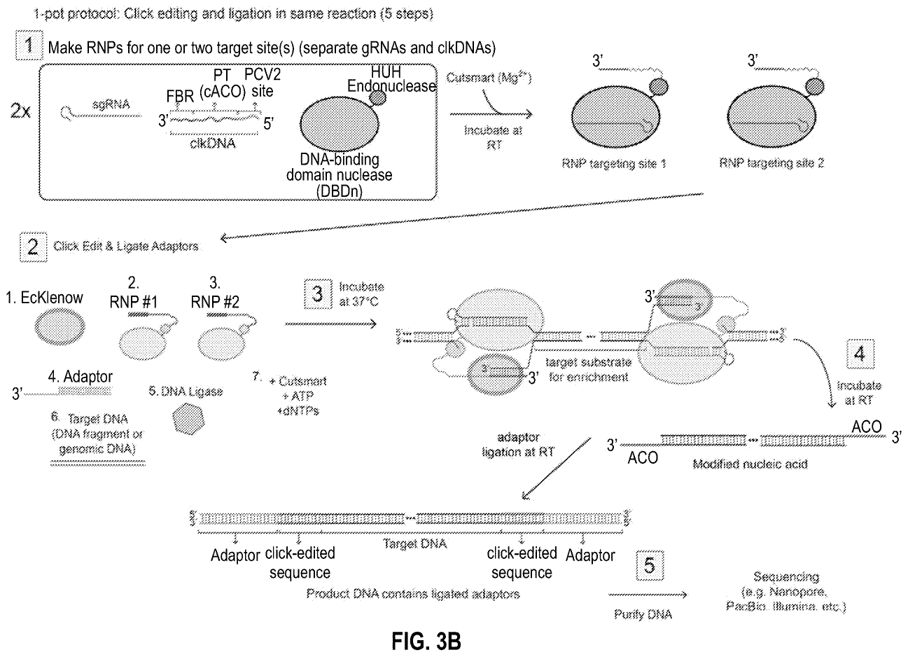

The sequence of the target DNA molecule (PCR substrate), which contains a chosen sgRNA sequence, is provided below. The 5-step, single-pot protocol proceeded as follows: (1) RNPs were formed using 100 pmol (1.1 μM final) sgRNA, 1× Cutsmart Buffer (New England Biolabs; NEB), and 48 pmol PCV2-Cas9 protein (0.53 μM final) and were incubated at room temperature for 20 min. 100 pmol of clkDNA encoding an ACO complementary sequence (cACO) was then added, and the mixture was incubated an additional 10 min at room temperature. (2) The CAGE reaction mix was assembled using 5 pmol of PCV2-Cas9 RNPs, 10 U of E. coli DNA Polymerase I Large Fragment (exo-) (NEB), 0.25 mM dNTPs, 1 mM dATP, and 1× CutSmart. 100 fmol of PCR target substrate (~70 ng) was then added to the reaction mixture. (3) The CAGE reaction was then incubated for 30-120 minutes (min) at 37° C. (click editing reaction). (4) Next, the reaction was incubated at 72° C. for 5 min (to inactivate the PCV2-Cas9 complex and dissociate Cas9 from bound DNA). (5) Finally, 400 U of T4 DNA ligase (New England Biolabs) and 500 fmol of the mock adaptor were added, and the mixture was incubated at room temperature for 20 min (FIG. 3A). A separate four step conception (with 2-steps of user intervention) of this protocol that lacks step #4, while adding the adaptor molecule at step #3 in the protocol rather than step #5, was also carried out (FIG. 3B). Reactions were bead purified with 0.8× beads, prepared as previously described^{9,10}.

Ligation efficiency was analyzed by several methods, including junction PCR (containing ~15 ng of template, 0.5 μM primers (Table 1), 0.4 U Q5 High-Fidelity DNA Polymerase (NEB), 0.5 μM dNTPs, that together were thermocycled at: 1 cycle, 98° C. for 3 min; 35 cycles, 98° C. for 10 sec, 66° C. for 15 sec, 72° C. for 20 sec; and hold at 4° C.), Sanger sequencing (sending approximately 100 ng PCR for sequencing), and ddPCR reactions (described below).

CAGE Reaction with Genomic DNA

For CAGE reactions targeting genomic DNA (gDNA), a similar protocol to that described above for PCR substrates was performed, with the following exceptions: 1 μg of human gDNA extracted from HEK 293T cells (extracted as previously described¹¹) was added to the CAGE reaction at step #2 instead of 70 ng of target DNA substrate, and a new set of gRNA and clkDNA specific for HEK site 3 was utilized. Click editing incubation time (step #3) was 120 min.

PE-CAGE Reaction with Genomic DNA

PE-CAGE reactions were performed using in vitro transcribed pegRNAs (transcribed using T7 RiboMax Express Large Scale RNA Production System; Promega) generated from a PCR templates that included a T7 promoter, an appropriate gRNA spacer and scaffold, and a 3′ extension including the primer binding site (PBS) and reverse transcriptase template (RTT) encoding the ACO to be installed at HEK site 3. In vitro cleavage and prime editing reactions were performed similar to as previously described¹², using 1 μg of HEK 293T genomic DNA, nuclease active SpCas9-HiFi (IDT), SuperScript III RT (ThermoFisher), the in vitro transcribed pegRNA, and the adaptor molecule. Reactions were incubated for 1 hr at 37° C.

ddPCR Analysis of Adaptor Ligation Efficiency

Ligation efficiency was assessed via ddPCR reactions containing 100-200 ng of human genomic DNA (for gDNA targeting experiments) or 10 μg of PCR target (for PCR-substrate targeting experiments), ddPCR Supermix for Probes (Bio-Rad), HindIII-HF (0.25 U μl⁻¹, New England Biolabs), RPP30 (for gDNA experiments) or PCR-substrate (for PCR-substrate experiments) control primers and probes (Table 1; 900 nM each primer, 250 nM probe), and target/adaptor specific primers and probes (Table 1; 900 nM each primer, 250 nM probe), according to the manufacturer's protocol. Droplets were generated using a QX200 Automated Droplet Generator (BioRad). Thermal cycling conditions were: 1 cycle, 95° C. for 10 min; 40 cycles, 94° C. for 30 sec, 58° C. for 1 min; 1 cycle, 98° C. for 10 min; and hold at 4° C. PCR products were analyzed using a QX200 Droplet Reader (BioRad) and the number of “adaptor-ligated” target copies and “total” copies (defined by RPP30 copies) was calculated using QuantaS oft (v.1.7.4). Adaptor ligation efficiency was defined as the ratio of adaptor-ligated target copies to total copies.

TABLE 1
Nucleotide Sequence List

| nucleotide description | nucleotide sequence |
|---|---|
| gRNA spacer for: | TAGTATAAACGAACAGGATC (SEQ ID NO: 75) |
| PCR_fragment_sgRNA | |
| gRNA spacer for: gDNA- | GGCCCAGACTGAGCACGTGA (SEQ ID NO: 76) |
| HEK3site_sgRNA | |
| pegRNA for HEK3 site spacer | GGCCCAGACTGAGCACGTGA (SEQ ID NO: 76) |
| pegRNA for HEK3 site | AGACGTGTGCTCTTCCGATCTCGTGCTCAGTCTG (SEQ |
| extension (RTT + PBS) | ID NO: 77) |
| adaptor_top-strand | TGAGTGGCCACACTCCGGAGTAACTAGTCATTGCCGAAG |
| (universal) | GTCTAG (SEQ ID NO: 78) |
| adaptor_bottom-strand | CTAGACCTTCGGCAATGACTAGTTACTCCGGAGTGTGGC |
| (universal) | CACTCAGCAACACCTCGGCTAGCATC (SEQ ID NO: |
| | 79) |
| PCR or ddPCR primer: | ATGGGACTTCAGCATGGCG (SEQ ID NO: 80) |
| control_PCR- | |
| amplicon_forward | |
| PCR or ddPCR primer: | ATCGCTGAGATAGGTGCCTC (SEQ ID NO: 81) |
| control_PCR- | |
| amplicon_reverse | |
| PCR or ddPCR primer: | ATGGGACTTCAGCATGGCG (SEQ ID NO: 80) |
| control_gDNA-RPP30_forward | |
| PCR or ddPCR primer: | TAGTTACTCCGGAGTGTGGCC (SEQ ID NO: 82) |
| control_gDNA-RPP30_reverse | |
| PCR or ddPCR primer: | TCCCCGAAAAGTGCCACCTG (SEQ ID NO: 83) |
| target_PCR-amplicon_forward | |
| PCR or ddPCR primer: | AGGGAAACGCCCATGCAATTAG (SEQ ID NO: 84) |
| target_gDNA-HEK3_forward | |
| PCR or ddPCR primer: | TAGTTACTCCGGAGTGTGGCC (SEQ ID NO: 82) |
| target_ADAPTOR_reverse | |
| (universal) | |
| PCR NGS p5 primer HEK3 | ACACTCTTTCCCTACACGACGCTCTTCCGATCTCCTAGA |
| | AAGGCATGGATGAGAGAAGC (SEQ ID NO: 85) |
| PCR NGS p7 primer poly-C | GACTGGAGTTCAGACGTGTGCTCTTCCGATCTCCCCCCC |
| | CCCCCCCCCCC (SEQ ID NO: 86) |
| ddPCR probe: | TCTGACCTGAAGGCTCTGCGCG (SEQ ID NO: 87) |
| PCR_control_probe (5′ HEX) | |
| ddPCR probe: | Acgttcgcgtttgccgtgcgtgtaatgtagtac (SEQ |
| PCR_target_probe (5′ FAM) | ID NO: 88) |
| ddPCR probe: | TCTGACCTGAAGGCTCTGCGCG (SEQ ID NO: 87) |
| RPP30_control_probe (5′ | |
| HEX) | |
| ddPCR probe: | CAAGCCTACAAATGCATGCTTACTTGCAG (SEQ ID |
| HEK3_target_probe (5′ FAM) | NO: 89) |
| clkDNA for: | CTGTAAGTATTACCAGCGCAACACCTCGGCTAGCATCCC |
| PCR_fragment_clkDNA | TGTTCGTTTATACT*A*G (SEQ ID NO: 90) |
| clkDNA for: HEK3_clkDNA | CTGTAAGTATTACCAGCGCAACACCTCGGCTAGCATCCG |
| | TGCTCAGTCTGGG*C*C (SEQ ID NO: 91) |

*denotes phosphorothioate linkages, PCV2 site (CTGTAAGTATTACCAGC (SEQ ID NO: 41)) is underlined

### Target PCR Fragment Sequence:

(SEQ ID NO: 92)
CATGGGACTTCAGCATGGCGGTGTTTGCAGATTACGCGAGCGGGTTC
TGACCTGAAGGCTCTGCGCGGATGAGTAAACTTGGTCTGACAGTTAC
CAATGCTTAATCAGTGAGGCACCTATCTCAGCGATCTGTCTATTTCG
TTCATCCATAGTTGCCTGACTCCCCGTCGTGTAGATAACTACGATAC
GGGAGGGCTTACCATCTGGCCCCAGTGCTGCAATGATACCGCGAGAC
CCACGCTCACCGGCTCCAGATTTATCAGCAATAAACCAGCCAGCCGG
AAGGGCCGAGCGCAGAAGTGGTCCTGCAACTTTATCCGCCTCCATCC
AGTCTATTAATTGTTGCCGGGAAGCTAGAGTAAGTAGTTCGCCAGTT
AATAGTTTGCGCAACGTTGTTGCCATTGCTACAGGCATCGTGGTGTC
ACGCTCGTCGTTTGGTATGGCTTCATTCAGCTCCGGTTCCCAACGAT
CAAGGCGAGTTACATGATCCCCCATGTTGTGCAAAAAAGCGGTTAGC
TCCTTCGGTCCTCCGATCGTTGTCAGAAGTAAGTTGGCCGCAGTGTT
ATCACTCATGGTTATGGCAGCACTGCATAATTCTCTTACTGTCATGC
CATCCGTAAGATGCTTTTCTGTGACTGGTGAGTACTCAACCAAGTCA
TTCTGAGAATAGTGTATGCGGCGACCGAGTTGCTCTTGCCCGGCGTC
AATACGGGATAATACCGCGCCACATAGCAGAACTTTAAAAGTGCTCA
TCATTGGAAAACGTTCTTCGGGGCGAAAACTCTCAAGGATCTTACCG
CTGTTGAGATCCAGTTCGATGTAACCCACTCGTGCACCCAACTGATC
TTCAGCATCTTTTACTTTCACCAGCGTTTCTGGGTGAGCAAAAACAG
GAAGGCAAAATGCCGCAAAAAAGGGAATAAGGGCGACACGGAAATGT
TGAATACTCATACTCTTCCTTTTTCAATATTATTGAAGCATTTATCA
GGGTTATTGTCTCATGAGCGGATACATATTTGAATGTATTTAGAAAA
ATAAACAAATAGGGGTTCCGCGCACATTTCCCCGAAAAGTGCCACCT
GACGTCGACGGATCGGACGTTCGCGTTTGCCGTGCGTGTAATGTAGT
ACTAGTATAAACGAACAGGATCAGGCCAAATTCCTGAATA

### Example 1. Click-Assisted Genome Enrichment (CAGE)

For our initial method we envisioned a single-pot workflow that consisted of five main steps with only three areas of user intervention: (1) RNP formation via PCV2-Cas9 protein incubation with the appropriate sgRNA and clkDNA, (2) Click editing reaction by mixing the PCE RNP with the target DNA substrate, EcKlenow, and reaction buffer (containing dNTPs, CutSmart buffer (NEB), and ATP), and (3) incubation of the mixture for 30-120 minutes (min) at 37° C. (click editing reaction), (4) incubation at 72° C. for 5 min (to inactivate the PCV2-Cas9 complex and dissociate Cas9 from bound DNA), (5) addition of T4 DNA ligase and the mock adaptor and then incubating at room temperature for 20 min, and (6) purification of the product prior to sequencing (FIG. 3A). A separate five step conception of this protocol that lacks step #4 (adding the adaptor molecule at step #2 in the protocol rather than step #5; FIG. 3B).

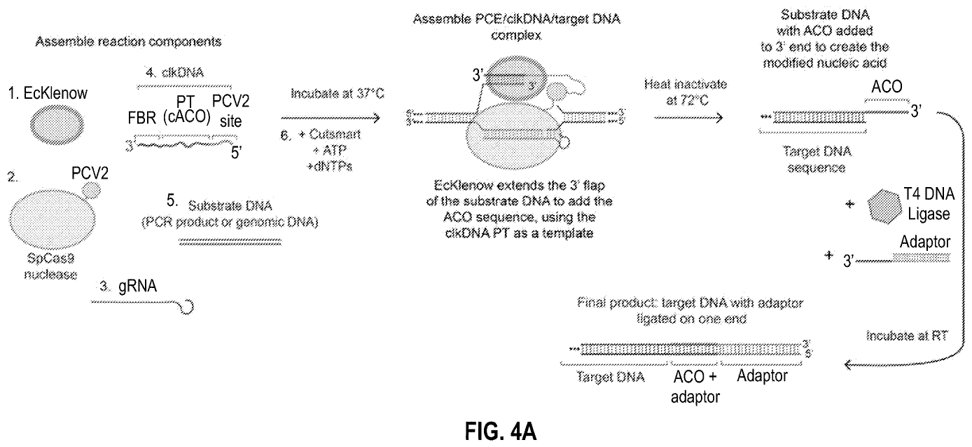

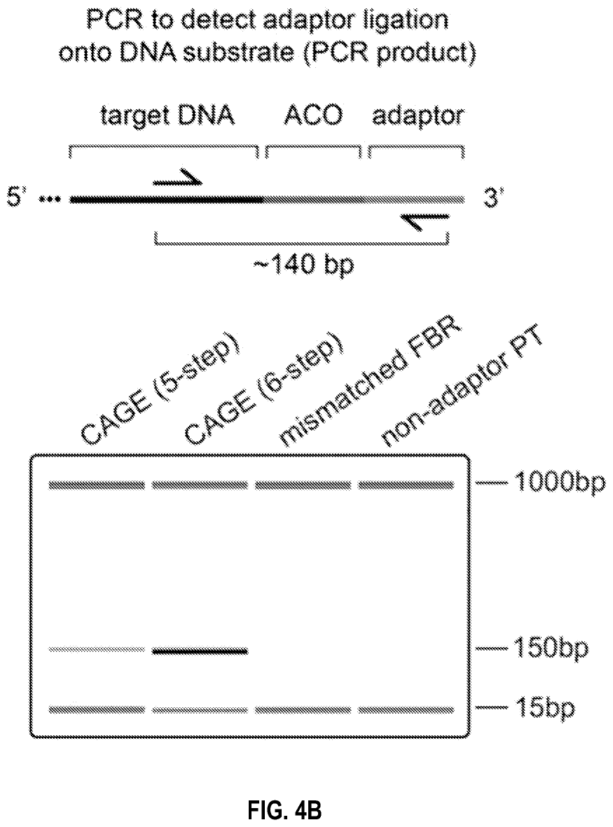

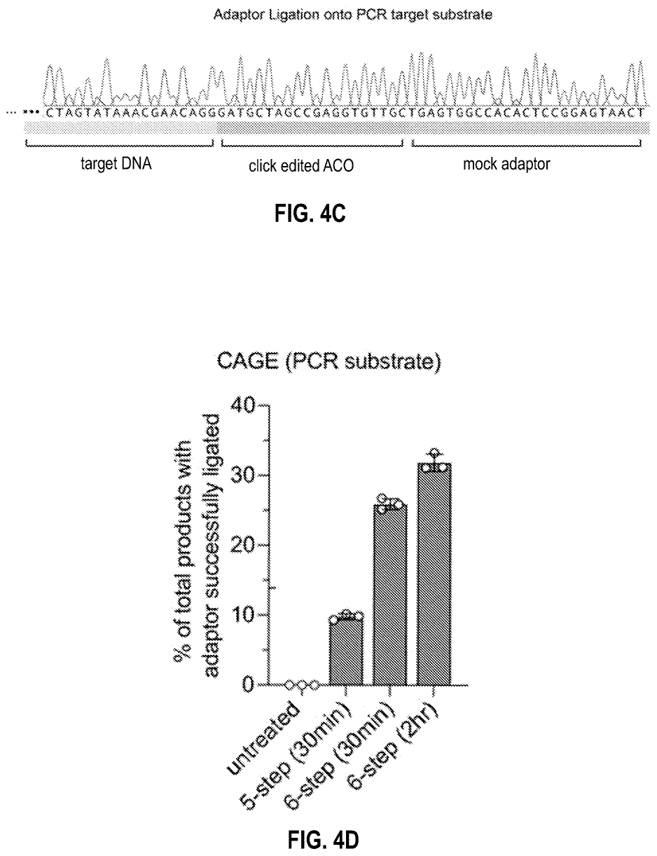

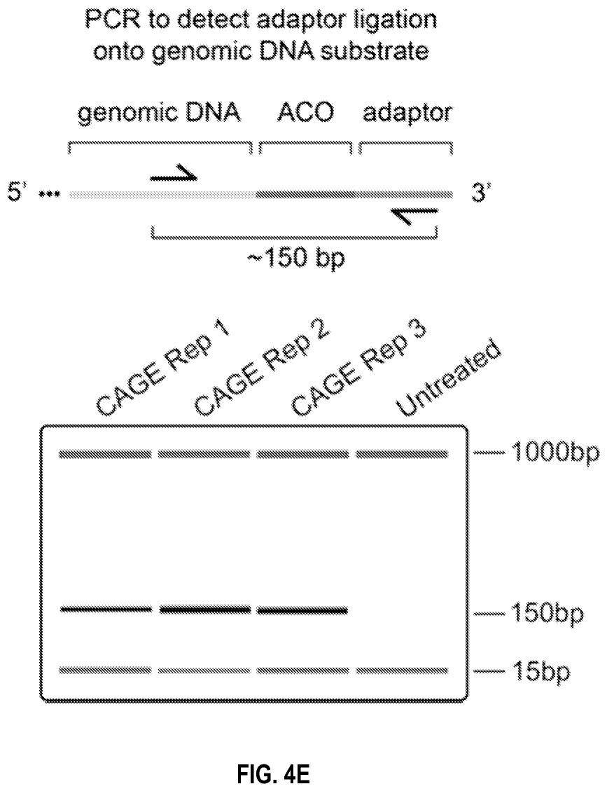

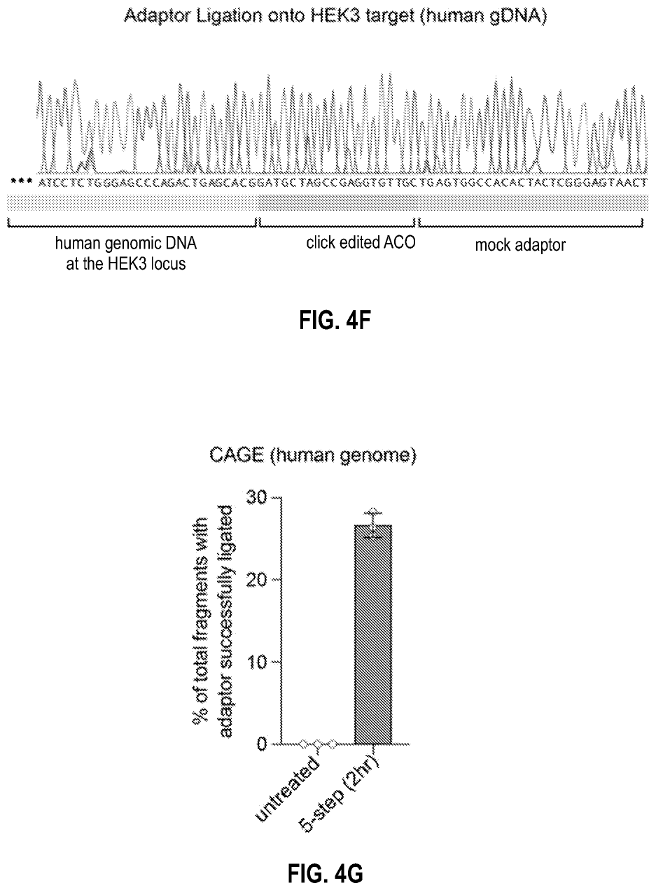

In experiments to test the initial ACO-writing step of a 6-step CAGE reaction on a single end of a substrate, we sought to ligate a mock adaptor onto a specific site of a DNA substrate in vitro (FIG. 4A). To do so, we purified an HUH-Cas9 fusion protein (comprised of the PCV2 HUH N-terminally fused to nuclease Streptococcus pyogenes Cas9 (SpCas9) via an 24aa gsXTENgs linker), purchased E. coli DNA Polymerase I Klenow Fragment (exo-) (EcKlenow) (New England Biolabs), designed a gRNA to target a specific site on a PCR substrate, and designed and ordered a clkDNA containing an appropriate FBR and a 20 nt PT (FIG. 4A). Successful polymerization by the PCE using the clkDNA in vitro should lead to the writing of a specific 20 nt 3′ overhang onto the NTS of the PCR-based DNA substrate at the SpCas9 cleavage site encoded on the target substrate (FIG. 4A). Lastly, we designed and constructed a mock adaptor that encodes a 20 nt sequence complementary to the ACO sequence on the target substrate.

To determine if the PCE-mediated ACO writing and adaptor ligation steps were successful, we conducted a PCR reaction using primers that should amplify across the overhang/ligation junction (where the forward primer is on the PCR substrate and the reverse primer is on the adaptor; FIG. 4B). Reactions containing the correct components led to a PCR product, indicating successful PCE-mediated ACO writing and adaptor ligation (FIG. 4B). Two separate control reactions did not result in PCR amplification, when using either a clkDNA with an incorrect FBR but correct PT (thus the clkDNA is not expected to anneal to the NTS), or a clkDNA encoding a correct FBR but incorrect PT (thus would be incompetent for adaptor ligation due to unmatched ACO) (FIG. 4B). We also found that a protocol lacking step #4 (incubation at 72° C. for 5 min; FIG. 3B) was also feasible, though at observably lower efficiency presumably due to target-bound Cas9 inhibiting adaptor ligation (FIG. 4B). Sanger sequencing of the adaptor-ligated PCR product demonstrated the expected product containing a correctly click-edited ACO installed at the precise location in the PCR substrate (due to Cas9 cleavage) followed by ligation of the adaptor sequence (FIG. 4C).

To determine the efficiency of ACO writing and adaptor ligation, we performed ddPCR on the CAGE reaction conducted using the 6-step protocol. We observed approximately 25.9% efficiency (FIG. 4D), which equates to approximately 20-million adaptor-ligated fragments in a 40 μL CAGE reaction. Increasing the click editing time from 30 min to 2 hours (during step #3 of the 6-step protocol) further increased efficiency to 31.8% (FIG. 4D). Like we observed in our junction PCR experiment (FIG. 4B), eliminating step #4 for a 5-step protocol (FIG. 3B) decreased adaptor ligation efficiency to 9.8% as determined by ddPCR (FIG. 4D).

Next, we wondered whether we could perform a CAGE reaction using genomic DNA instead of a PCR-generated substrate. To do so, we designed a PCE RNP and clkDNA to add an ACO to HEK site 3 using genomic DNA from HEK 293T cells (the same ACO as above from our PCR-based tests, permitting us to use the same mock adaptor for ligation; FIG. 4A). We followed the same protocol as our previous 6-step CAGE reaction with a 2 hour click editing incubation step using the same mock adaptor, but using 1 mg of human genomic DNA as the substrate (FIG. 4A). Junction PCR demonstrated successful adaptor ligation at the target site (FIG. 4E), sanger sequencing of the PCR product showed the expected sequence (HEK3 gDNA sequence—click edited overhang sequence—adaptor sequence; FIG. 4F), and ddPCR revealed approximately 26.6% ACO writing and adaptor ligation (FIG. 4G). These results reveal that CAGE is extensible to genomic DNA substrates.

Together, these results demonstrate that CAGE directs efficient installation of 3′ overhangs (ACOs) at user-defined target sequences in vitro, which can serve as specific and effective handles for ligation of adaptors containing a complementary 3′ overhang.

### Example 2. CAGE Reactions with Less Steps of User Intervention

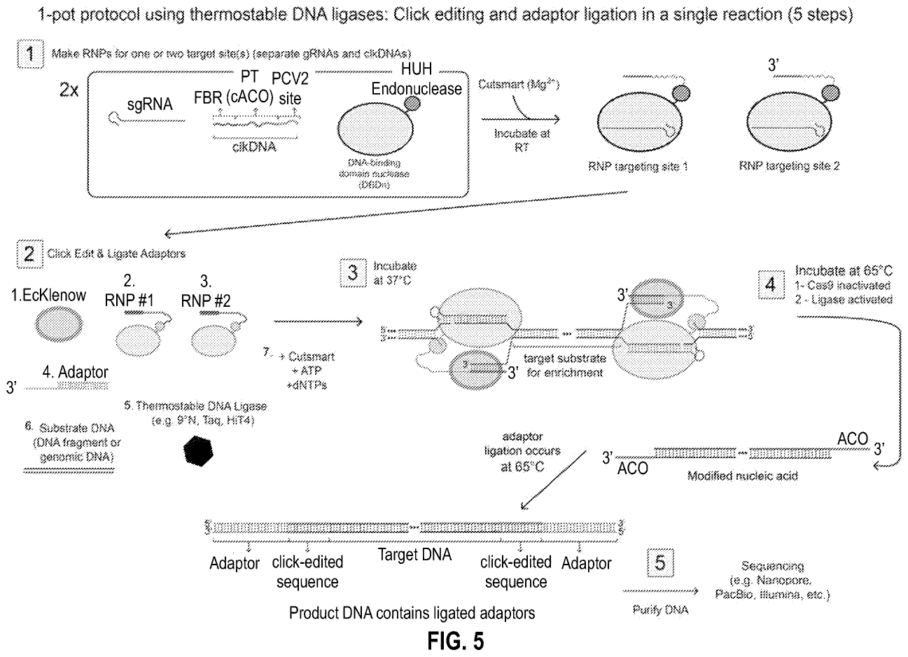

Minimizing user intervention would create a more streamlined workflow (FIG. 5). To do so, we imagined that thermostable ligases could enable the elimination of steps involving the manual addition of ligase and adaptor after Cas9 heat inactivation. Instead, all reagents (PCV2-Cas9 RNPs, EcKlenow, Ligase, adaptor, and buffer) could be mixed in a single tube and run on a thermocycler with an appropriate protocol (e.g. click editing at 37° C. for 30-120 min, followed by an elevated temperature at ~65° C. for 30-60 min). The incubation step at 65° C. should simultaneously inactivate/dissociate Cas9 while activating the ligase to complete the CAGE reaction.

### Example 3. Other Compositions of CAGE Using Prime Editors Instead of Click Editors

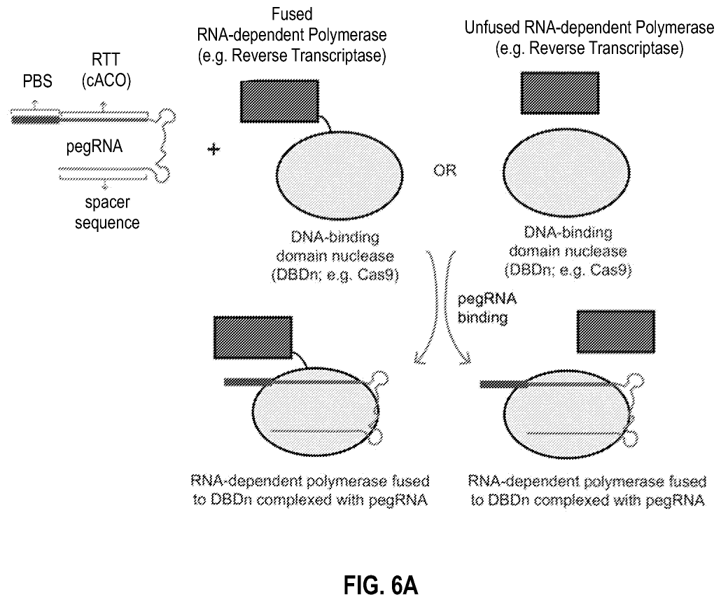

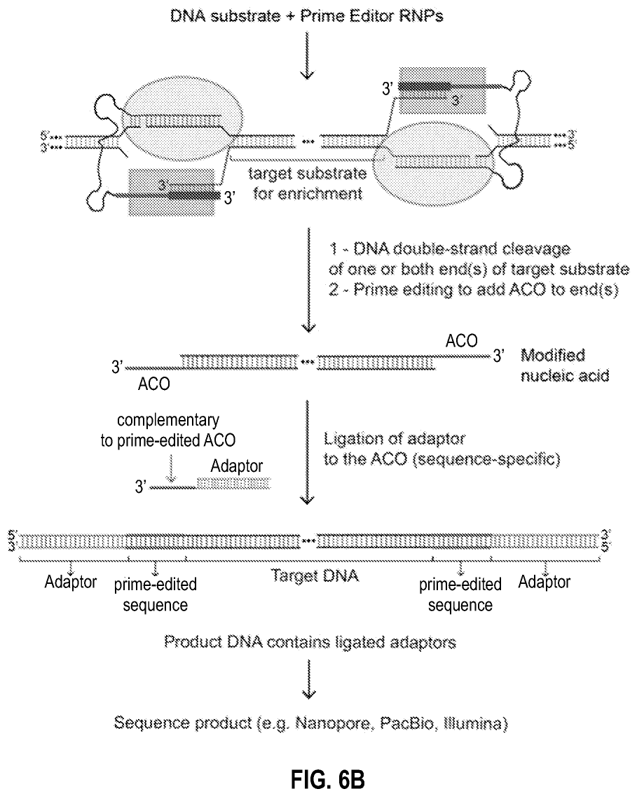

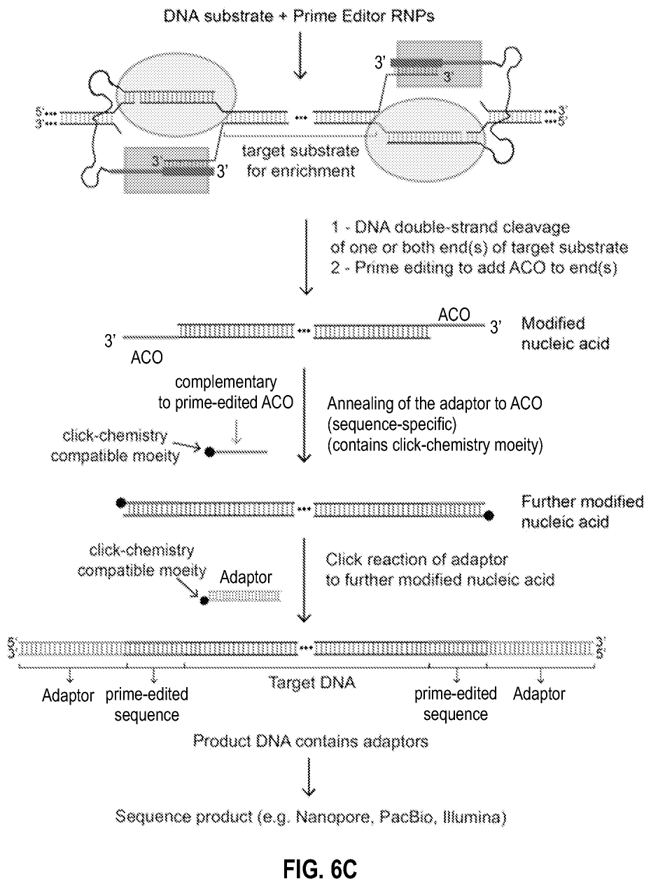

We also wondered whether CAGE could be performed using prime editors (PEs) instead of PCEs (in PE-CAGE reactions). PEs typically consist of a Cas9 nickase fused to or co-supplied with a RNA-dependent polymerase (e.g. reverse transcriptase (RT)) and an extended prime editing gRNA (pegRNA) that encodes a primer binding site (PBS) that anneals to the nicked NTS and a reverse transcription template (RTT) encoding the edit of interest¹² (FIG. 6A). For PE-CAGE, we envisioned using a Cas9 nuclease (instead of H840A nickase in conventional PEs) with a fused or unfused RT domain (FIG. 6A). During a prime editing reaction, the PE-extended 3′ flap from the NTS (using the RTT of the pegRNA as a template) would create an ACO, similar to how the PCE creates a 3′ ACO from the clkDNA. A PE-CAGE approach should be compatible with both modes of adaptor ligation described above for PCE-CAGE (FIGS. 6B, 6C, 10A, and 10B). We note a few potential complications of PE-CAGE. RT-mediated template extension past the RTT region of the pegRNA could lead to writing of nucleotides of the gRNA scaffold into the ACO (which could inhibit adaptor ligation). Furthermore, this method is potentially subject to increased cost if using synthetic pegRNAs (due to increased length and thus cost of the pegRNA compared to a conventional gRNA). However, PE-CAGE may hold advantages for applications that require extensive and simultaneous multiplexed enrichment within a given sample since the ACO template is inherently coupled to the gRNA in the pegRNA (compared to the challenge of complexing gRNA-clkDNA pairs together separately in arrayed reactions as would be needed for PCE-based approaches. PE RNPs can be formed with many pegRNAs in a single tube, whereas PCE RNPs must be complexed with clkDNAs separately to ensure correct linkage between target and ACO-writing template. We also note that PE-CAGE can be performed using an RNA-dependent RNA polymerase (RdRP), whereby an RNA ACO instead of a DNA ACO would be installed. A suitable ligase that ligates a 5′ DNA to a 3′ RNA on a DNA splint can be used (e.g., T4 RNA Ligase), leading to adaptor-ligated target products.

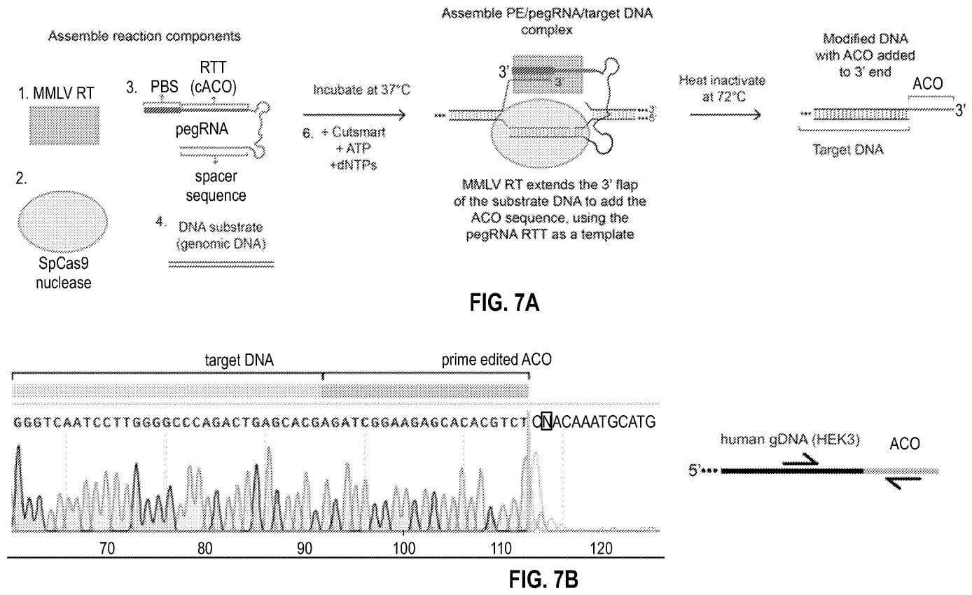

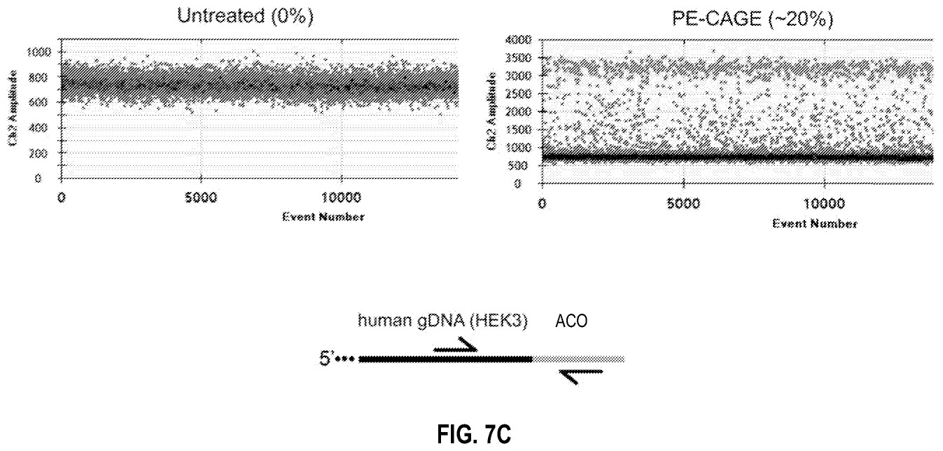

We performed a PE-CAGE reaction using nuclease active SpCas9-HiFi (IDT), SuperScript III RT provided in trans (ThermoFisher), an in vitro transcribed pegRNA (where the RTT portion encodes the template sequence for the ACO), human genomic DNA as a substrate and an adaptor complementary to the RTT of the pegRNA FIG. 7A). We performed the PE-CAGE reaction and to determine if the PE-mediated ACO writing was successful, PCR amplified the resulting product (using primers that should amplify across the ACO junction). Sanger sequencing revealed the anticipated product (FIG. 7B), confirming that Prime editing can install 3′ overhangs on target DNA molecules. We also performed ddPCR to quantify PE-mediated ACO extension, revealing approximately 20% ACO installation efficiency (FIG. 7C). These results demonstrate that PE-CAGE can also be used in protocols for selective labeling and enrichment of target nucleic acids.

Together, these results demonstrate that prime editing can also be used to efficiently install 3′ overhangs on target molecules in vitro, which could serve as sequence-specified handles for adaptor ligation. Identically to CAGE, PE-CAGE should enable at or near 100% enrichment, and improvements in efficiency (e.g. altering the sequence composition and length of the pegRNA RTT) could facilitate PE-CAGE enrichment from low-input samples. PE-CAGE has potential advantages over CAGE for applications requiring highly multiplexed targeting within a given sample. For example, since the RTT and sgRNA sequences are physically linked and a pool of pegRNAs can be complexed with a prime editor in a single tube (as opposed to CAGE, where clkDNA-sgRNA pairs would need to be complexed individually before pooling RNPs), multiple orthogonal PE-CAGE ACO-writing and adapter ligation steps should be possible simultaneously in a single sample. However, with PE-CAGE, adaptor ligation efficiency may be reduced from the heterogeneity of ACO writing (due to RT-mediated extension past the RTT into the sgRNA scaffold, which would add unwanted bases to the ACO and thus preclude adaptor ligation). Furthermore, the price per reaction would be higher if using synthetic pegRNAs. However, extraneous ACO extension in PE-CAGE could be minimized by: (1) screening RNA hairpins that could be inserted into the pegRNA scaffold at the end of the RTT, which might terminate reverse transcription, (2) using a pool of adaptors containing overhangs which account for scaffold read-through, (3) by using synthetic modified pegRNAs that inhibit extension past the RTT (e.g. via use of a chemical linker or abasic site between the RTT and sgRNA scaffold, etc.), or (4) by using split pegRNAs where the RTT/PBS molecule is separated from the gRNA.

We anticipate that the enzymes or enzyme fusions used in PE-CAGE reactions may also comprise a variety of architectures, including a nuclease (N) for cleaving the substrate and an RNA-dependent polymerase (P) (e.g. a reverse-transcriptase; RT) for extending the template, separated by optional linkers (L). Although herein we utilized a separated nuclease and polymerase architecture (N+P), where the nuclease/DNA binding domain was SpCas9 and the polymerase was MMLV-RT, we envision alternate architectures beyond N+P that include but are not limited to: N-L-P or P-L-N (where N is the nuclease, P is the polymerase, and L is a linker; each of varying compositions). The nuclease domain (N) may be SpCas9 or other nucleases; the RNA-dependent polymerase domain (P) may be MMLV reverse-transcriptase or other polymerases; and the linkers (L) may be of various lengths and amino acid compositions.

### Example 4. Click-Assisted Precise Targeting of Unaltered RNA for Enrichment (CAPTURE)

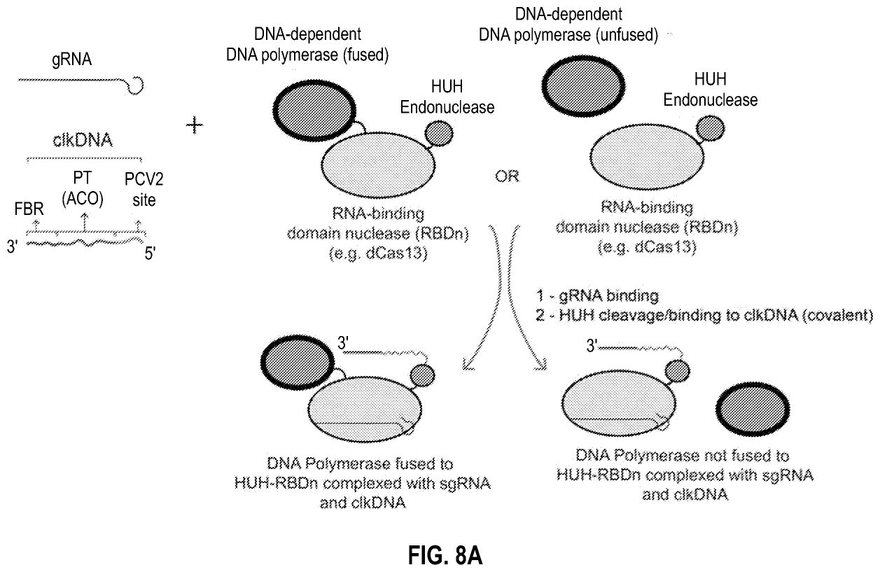

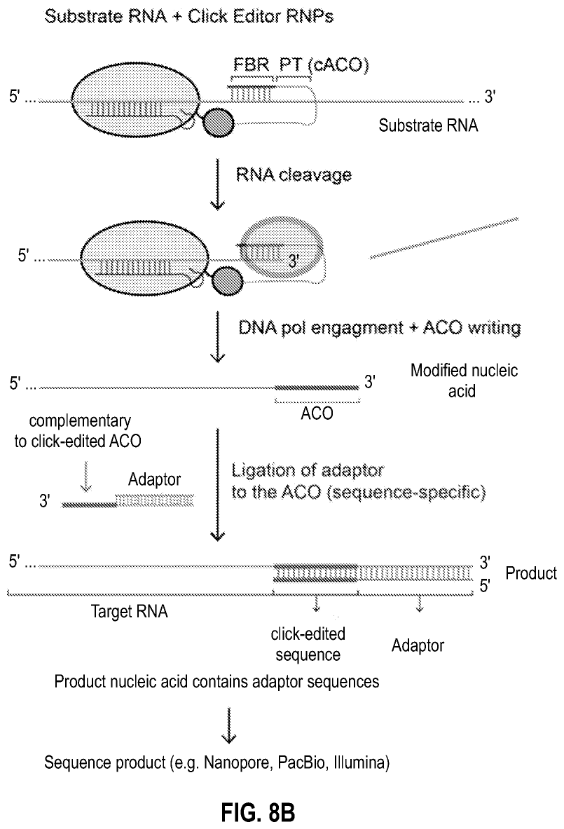

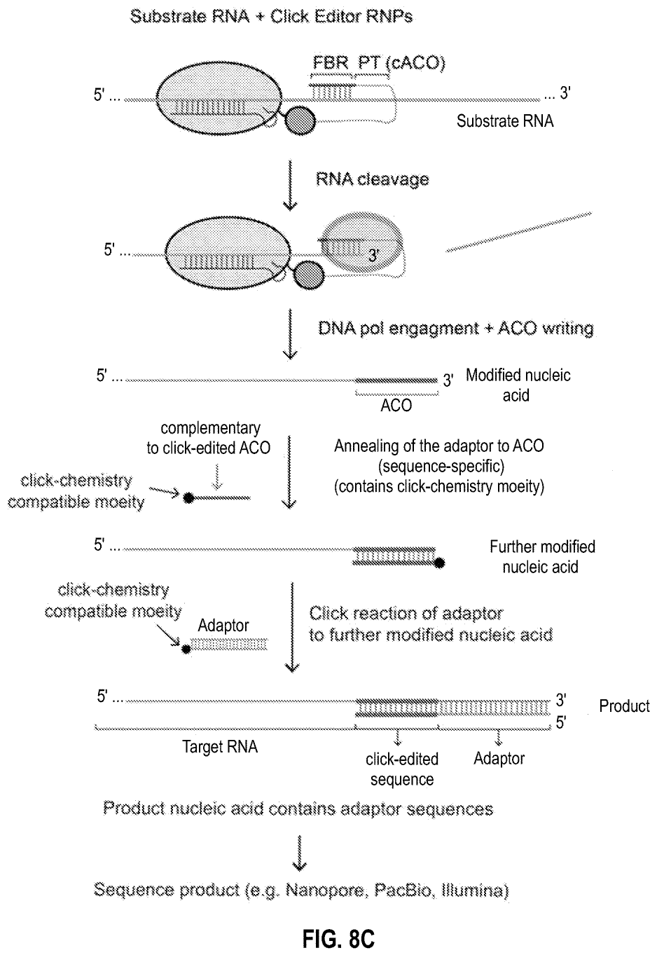

We also envisioned that the process of targeted overhang installation could be applied to RNA substrates to enrich for user-specifiable RNA molecules or transcripts from a pool without amplification or cDNA conversion (FIG. 8). To do so, we hypothesized that substituting PCV2-Cas9 for PCV2-Cas13 (ideally, a high fidelity Cas13 variant that minimizes or eliminates unwanted collateral cleavage of nearby non-target RNA molecules¹³) would enable precise RNA transcript targeting and enrichment as defined by the Cas13 gRNA (FIG. 8A). Cleavage of the target RNA molecule at the user-specified gRNA target site would create an RNA primer that though annealing with the FBR of the clkDNA would create an RNA:DNA substrate for extension by a DNA-dependent polymerase (e.g. EcKlenow) (FIGS. 8B, 8C, 10C, and 10D). Dissociation of the PCV2-Cas13 RNP from the target RNA would leave a hybrid target RNA:DNA molecule tagged on the 3′ end with a DNA ACO whose sequence is defined by the PT of the clkDNA. As with CAGE, this DNA ACO could then be used as a sequence for annealing and ligating specific adaptor. The adaptor ligated and samples can then be further processed or directly loaded onto a sequencing platform (e.g. a nanopore sequencer).

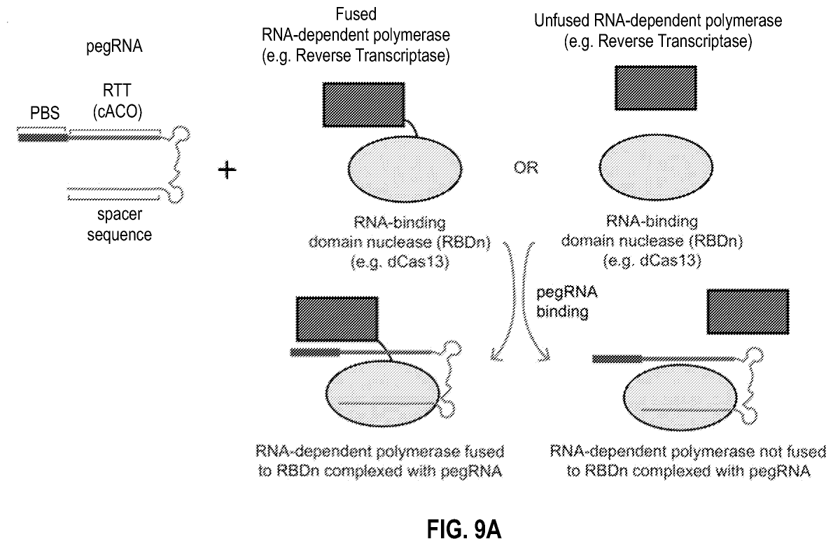

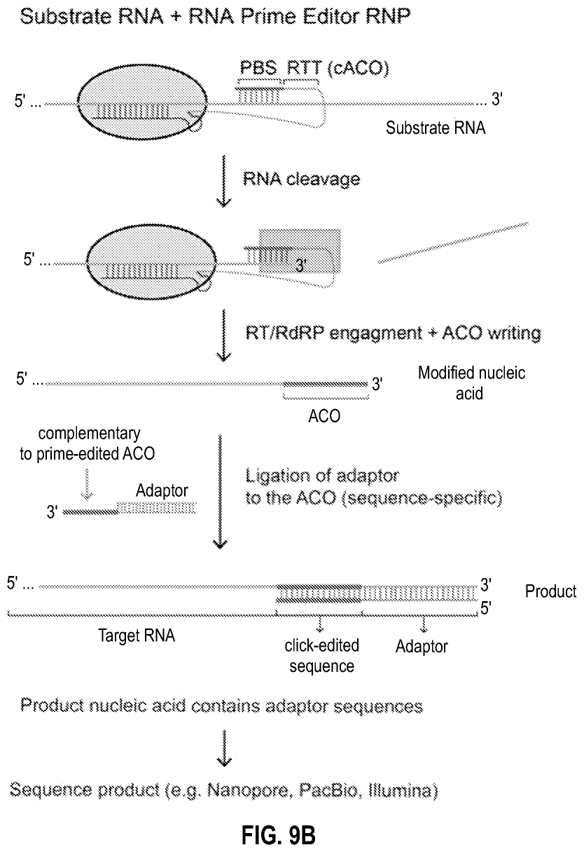

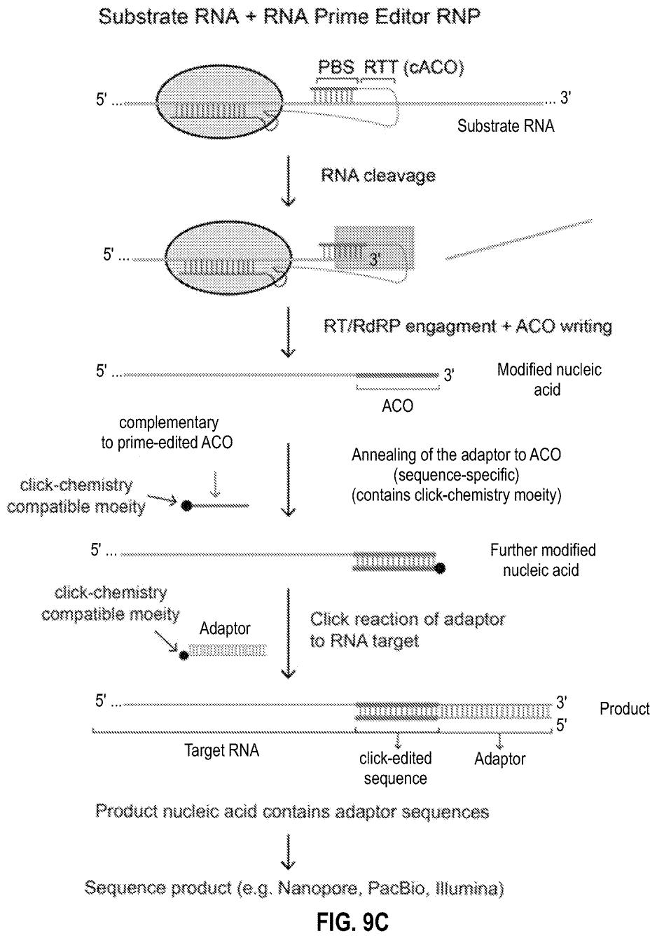

CAPTURE could also conceivably be performed with a prime editing like system (PE-CAPTURE), where Cas13 nuclease can be fused to or co-supplied with a reverse transcriptase (or RdRP) and the Cas13 gRNA can be extended at the 3′ or 5′ end to include a PBS and an RTT containing the overhang sequence (FIGS. 9A-9C, 10C, and 10D).

### Example 5. CAGE Using Cas9 Nuclease and No ssDNA Tethering Domain

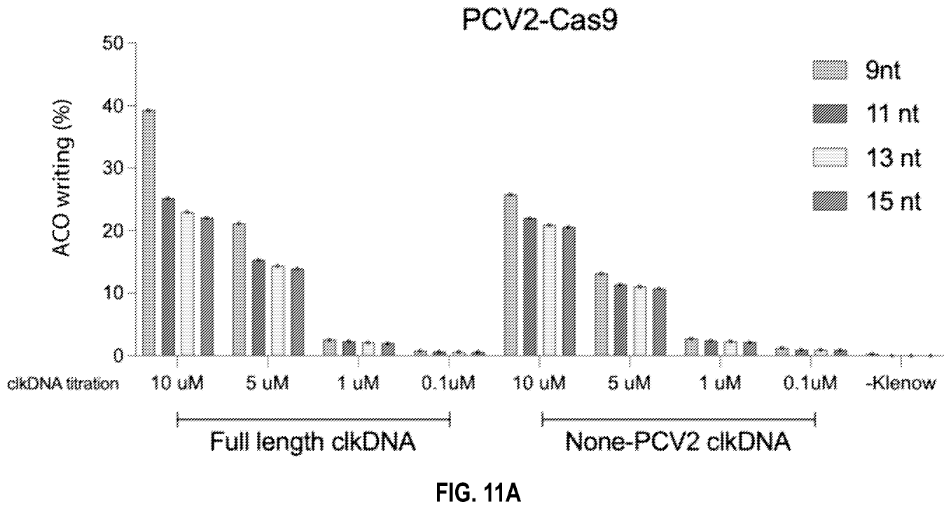

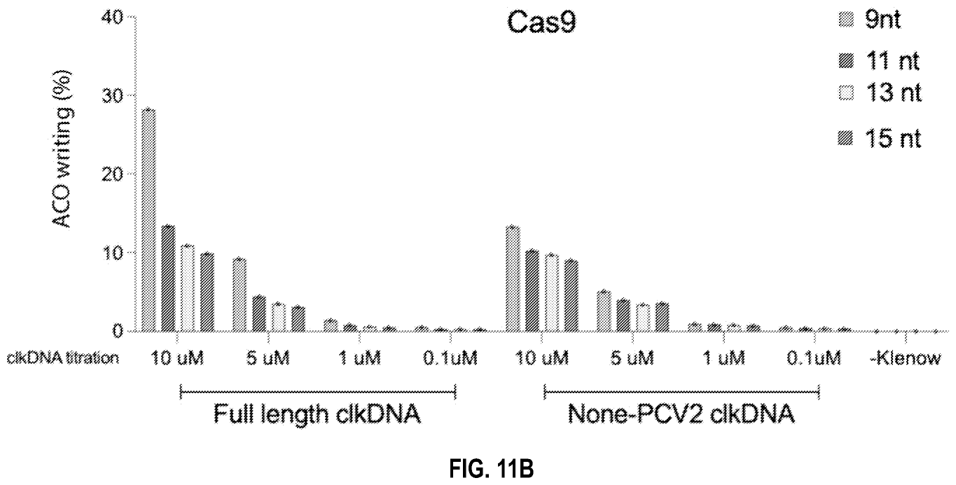

To investigate whether a tethering domain (such as PCV2) was essential, we conducted CAGE reactions on a PCR substrate with varying amounts of clkDNA containing or lacking a PCV2 recognition sequence and with PCV2-Cas9 or Cas9 alone (with no tethering domain). Reactions (containing cleaved PCR products with written flaps or cleaved products without flaps) were dG-tailed and amplified using a poly-C reverse primer and a junction specific forward primer. While the combination of PCV2-Cas9 and a clkDNA containing the PCV2 recognition sequence was generally most efficient (measured by ddPCR), the inclusion of the PCV2 sequence alone was generally beneficial and CAGE could be conducted with Cas9 alone (no PCV2 endonuclease) (FIGS. 11A-B). Similar trends were also observed on a gDNA substrate at the HEK3 locus.

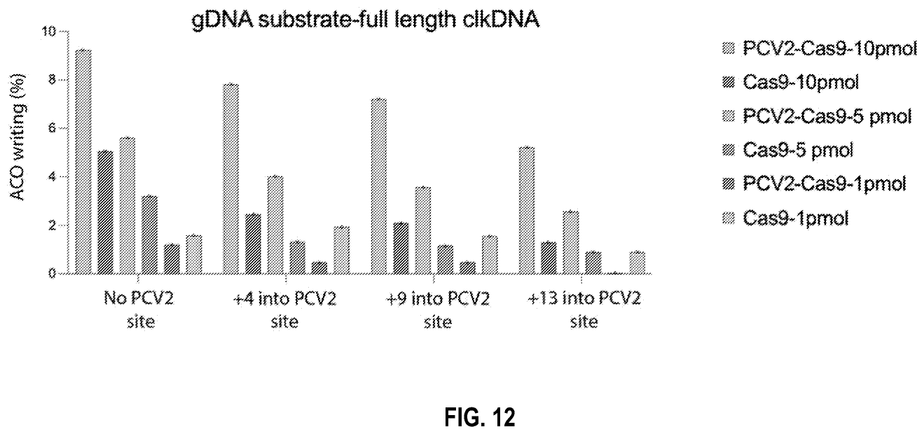

On both PCR and genomic substrates, we also observed truncation products when using wild type E. coli Klenow fragment (FIGS. 11A-B), which may be due to its relatively low processivity and/or difficulty reading through secondary template structure. We also observed polymerization of the PCV2 recognition sequence-likely from unbound clkDNA during RNP formation (FIG. 12).

### REFERENCES

1. Kozarewa, I., Armisen, J., Gardner, A. F., Slatko, B. E. & Hendrickson, C. L. Overview of Target Enrichment Strategies. CP Molecular Biology 112, (2015).
2. Karamitros, T. & Magiorkinis, G. Multiplexed Targeted Sequencing for Oxford Nanopore MinION: A Detailed Library Preparation Procedure. in Next Generation Sequencing (eds. Head, S. R., Ordoukhanian, P. & Salomon, D. R.) vol. 1712 43-51 (Springer New York, 2018).
3. Leija-Salazar, M. et al. Evaluation of the detection of GBA missense mutations and other variants using the Oxford Nanopore MinION. Mol Genet Genomic Med 7, e564 (2019).
4. Gabrieli, T. et al. Selective nanopore sequencing of human BRCA1 by Cas9-assisted targeting of chromosome segments (CATCH). Nucleic Acids Research 46, e87-e87 (2018).
5. Wallace, A. D. et al. CaBagE: A Cas9-based Background Elimination strategy for targeted, long-read DNA sequencing. PLOS ONE 16, e0241253 (2021).
6. Iyer, S. V., Kramer, M., Goodwin, S. & McCombie, W. R. ACME: an Affinity-based Cas9 Mediated Enrichment method for targeted nanopore sequencing. 2022.02.03.478550 Preprint at https://doi.org/10.1101/2022.02.03.478550 (2022).
7. Gilpatrick, T. et al. Targeted nanopore sequencing with Cas9-guided adapter ligation. Nat Biotechnol 38, 433-438 (2020).
8. Anders, C. & Jinek, M. In Vitro Enzymology of Cas9. in Methods in Enzymology vol. 546 1-20 (Elsevier, 2014).
9. Rohland, N. & Reich, D. Cost-effective, high-throughput DNA sequencing libraries for multiplexed target capture. Genome Res. 22, 939-946 (2012).
10. Kleinstiver, B. P. et al. Engineered CRISPR-Cas12a variants with increased activities and improved targeting ranges for gene, epigenetic and base editing. Nat Biotechnol 37, 276-282 (2019).
11. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
12. Anzalone, A. V. et al. Search-and-replace genome editing without double-strand breaks or donor DNA. Nature 576, 149-157 (2019).
13. Tong, H. et al. High-fidelity Cas13 variants for targeted RNA degradation with minimal collateral effect. 2021.12.18.473271 https://www.biorxiv.org/content/10.1101/2021.12.18.473271v1 (2021) doi:10.1101/2021.12.18.473271.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
preparing a reaction mixture comprising:
(i) a DNA binding domain nuclease (DBDn), optionally Cas9, optionally linked or fused to a clkDNA tethering domain;
(ii) one, two, or more guide RNAs (gRNAs) that bind to the DBDn and comprise a spacer comprising a sequence complementary to a selected target sequence;
(iii) one, two, or more clkDNA oligonucleotide templates comprising a localization moiety that binds to the clkDNA tethering domain, a template encoding the complement of an adaptor-complementary overhang (ACO) sequence (cACO), and a flap binding region (FBR), and
optionally (iv) a DNA-dependent DNA polymerase, optionally wherein the polymerase is linked or fused to the DBDn in the reaction mixture;
wherein (i)-(iii) or (i)-(iv) are added to the mixture in any order, and incubating the reaction mixture under conditions for the gRNAs to bind to the DBDn and the clkDNA oligonucleotide templates to bind to the clkDNA tethering domain on the DBDn, and optionally wherein the reaction mixture further comprises:
(iv) a substrate nucleic acid comprising a region of interest.

2. A method for generating a modified nucleic acid of DNA comprising 3′ overhangs comprising an adaptor-complementary overhang (ACO) sequence, the method comprising:
(A) providing a reaction mixture prepared by the method of claim 1, comprising:
(i) a DNA binding domain nuclease (DBDn), optionally Cas9, optionally linked or fused to a clkDNA tethering domain;
(ii) one, two, or more guide RNAs (gRNAs) that bind to the DBDn and comprise a spacer comprising a sequence complementary to a selected target sequence; and
(iii) one, two, or more clkDNA oligonucleotide templates comprising a localization moiety that binds to the clkDNA tethering domain, a template encoding the complement of an adaptor-complementary overhang (ACO) sequence (cACO), and a flap binding region (FBR),
wherein (i)-(iii) are added to the mixture in any order, and
incubating the reaction mixture under conditions for the gRNAs to bind to the DBDn and the clkDNA oligonucleotide templates to bind to the clkDNA tethering domain on the DBDn;
(B) providing a sample comprising a substrate nucleic acid of DNA, preferably genomic DNA isolated from a cell; and
contacting the sample with the reaction mixture of (A) and a DNA-dependent DNA polymerase if the polymerase is absent from the reaction mixture, incubating the sample under conditions wherein:
the nuclease induces a pair of double-stranded breaks with 3′ single stranded flaps;
the clkDNA oligonucleotide templates bind to the 3′ single stranded flaps; and
the polymerase extends the 3′ single stranded flaps using the cACO portion of the clkDNA as a template, thereby producing a modified nucleic acid of DNA comprising 3′ overhangs comprising an adaptor-complementary overhang (ACO) sequence.

3. A method of generating a product nucleic acid of DNA having ends comprised of defined adaptor or overhang sequences, the method comprising:
providing a sample comprising a modified nucleic acid of DNA comprising 3′ overhangs comprising an ACO sequence produced by the method of claim 2;
optionally treating the sample to inactivate any active enzymes, optionally by heating the sample to above 60° C., optionally to about 72° C.;
contacting the modified nucleic acid of DNA with a partially double stranded DNA oligonucleotide comprising defined adaptor sequences and a 3′ overhang comprising a sequence complementary to the ACO sequence (dsDNA adapter with ssDNA cACO region) and a ligase, under conditions sufficient for the ligase to ligate the adapter to the modified nucleic acid, thereby producing a product nucleic acid of DNA having ends comprised of defined adaptor sequences.

4. A method of generating a product nucleic acid of DNA having ends comprised of defined adaptor sequences, the method comprising:
providing a modified nucleic acid of DNA comprising 3′ overhangs at comprising ACO sequence produced by the method of claim 2;
contacting the modified nucleic acid of DNA with a ligase and a single stranded DNA oligonucleotide modified with a click-compatible moiety at the 5′ end (ssDNA click oligo) comprising a sequence complementary to the ACO sequence under conditions sufficient for the ssDNA click oligo to anneal to the 3′ overhangs and be ligated to the modified nucleic acid of DNA, thereby producing a further modified nucleic acid of DNA having 5′ ends comprising click-compatible moieties; and
contacting the further modified nucleic acid of DNA having 5′ ends comprising click-compatible moieties with dsDNA click oligos comprising defined adaptor sequences and a 3′ click compatible moiety that reacts with the click-compatible moieties on the further modified nucleic acid of DNA to attach the dsDNA click oligos to the ends of the further modified nucleic acid of DNA, thereby producing a product nucleic acid of DNA having ends comprised of defined adaptor sequences.

5. A method for generating a product nucleic acid of DNA having ends comprised of defined adaptor sequences, the method comprising:
(A) providing a reaction mixture prepared by the method of claim 1, comprising:
(i) a DNA binding domain nuclease (DBDn), optionally Cas9, optionally linked or fused to a clkDNA tethering domain;
(ii) one, two, or more guide RNAs (gRNAs) that bind to the DBDn and comprise a spacer comprising a sequence complementary to a selected target sequence; and
(iii) one, two, or more clkDNA oligonucleotide templates comprising a localization moiety that binds to the clkDNA tethering domain, a template encoding the complement of an adaptor-complementary overhang (ACO) sequence (cACO), and a flap binding region (FBR),
wherein (i)-(iii) are added to the mixture in any order, and
incubating the reaction mixture under conditions for the gRNAs to bind to the DBDn and the clkDNA oligonucleotide templates to bind to the clkDNA tethering domain on the DBDn;
(B) providing a sample comprising a substrate nucleic acid of DNA, preferably genomic DNA isolated from a cell; and
contacting the sample with:
the reaction mixture of (A),
a DNA-dependent DNA polymerase if the polymerase is absent from the reaction mixture,
a double stranded DNA oligonucleotide comprising defined adaptor sequences and a 3′ overhang comprising a sequence complementary to the ACO sequence (dsDNA cACO oligo), and
a ligase, optionally a thermostable ligase,
incubating the sample under conditions wherein:
the nuclease induces a pair of double-stranded breaks with 3′ single stranded flaps;
the clkDNA oligonucleotide templates bind to the 3′ single stranded flaps;
the polymerase extends the 3′ single stranded flaps using the ACO complementary portion of the clkDNA as a template, thereby producing a modified nucleic acid of DNA comprising 3′ overhangs comprising an adaptor-complementary overhang (ACO) sequence, and
incubating the sample under conditions wherein the thermostable ligase ligates the dsDNA oligo to the modified nucleic acid of DNA, thereby producing a product nucleic acid of DNA having ends comprised of defined adaptor sequences.

6. A method for generating a product nucleic acid of DNA having ends comprised of defined adaptor sequences, the method comprising:
(A) providing a reaction mixture prepared by the method of claim 1, comprising:
(i) a DNA binding domain nuclease (DBDn), optionally cas9, optionally linked or fused to a clkDNA tethering domain;
(ii) one, two, or more guide RNAs (gRNAs) that bind to the DBDn and comprise a spacer comprising a sequence complementary to a selected target sequence; and
(iii) one, two, or more clkDNA oligonucleotide templates comprising a localization moiety that binds to the clkDNA tethering domain, a template encoding the complement of an adaptor-complementary overhang (ACO) sequence (cACO), and a flap binding region (FBR),
wherein (i)-(iii) are added to the mixture in any order, and
incubating the reaction mixture under conditions for the gRNAs to bind to the DBDn and the clkDNA oligonucleotide templates to bind to the clkDNA tethering domain on the DBDn;
(B) providing a sample comprising a substrate nucleic acid of DNA, preferably genomic DNA isolated from a cell; and
contacting the sample with:
the reaction mixture of (A),
a DNA-dependent DNA polymerase if the polymerase is absent from the reaction mixture,
a single stranded DNA oligonucleotide modified with a click-compatible moiety at the 5′ end (ssDNA click oligo) comprising a sequence complementary to the ACO sequence, and
a thermostable ligase,
incubating the sample under conditions wherein:
the nuclease induces a pair of double-stranded breaks with 3′ single stranded flaps;
the clkDNA oligonucleotide templates bind to the 3′ single stranded flaps;
the polymerase extends the 3′ single stranded flaps using the ACO complementary portion of the clkDNA as a template, thereby producing a modified fragment of DNA comprising 3′ overhangs comprising an adaptor-complementary overhang (ACO) sequence, and
incubating the sample under conditions wherein the thermostable ligase is active, for the ssDNA click oligo to anneal to the 3′ overhangs and be ligated to the modified nucleic acid of DNA, thereby producing a further modified nucleic acid of DNA having 5′ ends comprising click-compatible moieties; and
contacting the further modified nucleic acid of DNA having 5′ ends comprising click-compatible moieties with dsDNA click oligos comprising defined adaptor sequences and a 3′ click compatible moiety that reacts with the click-compatible moieties on the further modified nucleic acid of DNA to attach the dsDNA click oligos to the ends of the further modified nucleic acid of DNA, thereby producing a product nucleic acid of DNA having ends comprised of defined adaptor sequences.

7. A method comprising:
preparing a reaction mixture comprising:
(i) a DNA binding domain nuclease (DBDn), optionally Cas9;
(ii) one, two, or more pegRNAs that bind to the DBDn and comprise a 3′ extension including a primer binding site (PBS) a reverse transcriptase template (RTT) encoding the complement of the ACO, a scaffold and spacer comprising a sequence complementary to a selected target sequence; and
optionally (iii) an RNA-dependent polymerase, optionally wherein the polymerase is linked or fused to the DBDn in the reaction mixture;
wherein (i)-(ii) or (i)-(iii) are added to the mixture in any order, and incubating the reaction mixture under conditions for the gRNAs to bind to the DBDn.

8. A method for generating a modified nucleic acid of DNA comprising 3′ overhangs comprising an adaptor-complementary overhang (ACO) sequence, the method comprising:
(A) providing a reaction mixture prepared according to claim 7, comprising (i) a DNA binding domain nuclease (DBDn), optionally Cas9;
(ii) one, two, or more pegRNAs that bind to the DBDn and comprise a 3′ extension including a primer binding site (PBS) a reverse transcriptase template (RTT) encoding the complement of the ACO, a scaffold and spacer comprising a sequence complementary to a selected target sequence; and
optionally (iii) an RNA-dependent polymerase, optionally wherein the polymerase is linked or fused to the DBDn in the reaction mixture;
wherein (i)-(ii) or (i)-(iii) are added to the mixture in any order, and incubating the reaction mixture under conditions for the gRNAs to bind to the DBDn;
(B) providing a sample comprising a substrate nucleic acid of DNA, preferably genomic DNA isolated from a cell; and
contacting the sample with the reaction mixture of (A) and an RNA-dependent DNA polymerase if the polymerase is absent from the reaction mixture, incubating the sample under conditions wherein:
the nuclease induces a pair of double-stranded breaks with 3′ single stranded flaps;
the clkDNA oligonucleotide templates bind to the 3′ single stranded flaps; and
the polymerase extends the 3′ single stranded flaps using the ACO complementary portion of the clkDNA as a template, thereby producing a modified nucleic acid of DNA comprising 3′ overhangs comprising an adaptor-complementary overhang (ACO) sequence.

9. A method of generating a product nucleic acid of DNA having ends comprised of defined adaptor sequences, the method comprising:
providing a sample comprising a modified nucleic acid of DNA comprising 3′ overhangs at comprising ACO sequence produced by the method of claim 8;
optionally treating the sample to inactivate any active enzymes, optionally by heating the sample to about 72° C.;
contacting the modified nucleic acid of DNA with a double stranded DNA oligonucleotide comprising defined adaptor sequences and a 3′ overhang comprising a sequence complementary to the ACO sequence (dsDNA cACO oligo) and a ligase, under conditions sufficient for the ligase to ligate the dsDNA oligo to the modified nucleic acid of DNA, thereby producing a product nucleic acid of DNA having ends comprised of defined adaptor sequences.

10. A method of generating a product nucleic acid of DNA having ends comprised of defined adaptor sequences, the method comprising:
providing a modified nucleic acid of DNA comprising 3′ overhangs at comprising ACO sequence produced by the method of claim 8;
contacting the modified nucleic acid of DNA with a ligase and a single stranded DNA oligonucleotide modified with a click-compatible moiety at the 5′ end (ssDNA click oligo) comprising a sequence complementary to the ACO sequence under conditions sufficient for the ssDNA click oligo to anneal to the 3′ overhangs and be ligated to the modified nucleic acid of DNA, thereby producing a further modified nucleic acid of DNA having 5′ ends comprising click-compatible moieties; and
contacting the further modified nucleic acid of DNA having 5′ ends comprising click-compatible moieties with dsDNA click oligos comprising defined adaptor sequences and a 3′ click compatible moiety that reacts with the click-compatible moieties on the further modified nucleic acid of DNA to attach the dsDNA click oligos to the ends of the further modified nucleic acid of DNA, thereby producing a product nucleic acid of DNA having ends comprised of defined adaptor sequences.

11. A method for generating a product nucleic acid of DNA having ends comprised of defined adaptor sequences, the method comprising:
(A) providing a reaction mixture prepared by the method of claim 7, comprising
(i) a DNA binding domain nuclease (DBDn), optionally Cas9;
(ii) one, two, or more pegRNAs that bind to the DBDn and comprise a 3′ extension including a primer binding site (PBS) a reverse transcriptase template (RTT) encoding the complement of the ACO, a scaffold and spacer comprising a sequence complementary to a selected target sequence; and
optionally (iii) an RNA-dependent polymerase, optionally wherein the polymerase is linked or fused to the DBDn in the reaction mixture;
wherein (i)-(ii) or (i)-(iii) are added to the mixture in any order, and incubating the reaction mixture under conditions for the gRNAs to bind to the DBDn;
(B) providing a sample comprising a substrate nucleic acid of DNA, preferably genomic DNA isolated from a cell; and
contacting the sample with:
the reaction mixture of (A),
a RNA-dependent DNA polymerase if the polymerase is absent from the reaction mixture,
a double stranded DNA oligonucleotide comprising defined adaptor sequences and a 3′ overhang comprising a sequence complementary to the ACO sequence (dsDNA cACO oligo), and
a ligase, optionally a thermostable ligase,
incubating the sample under conditions wherein:
the nuclease induces a pair of double-stranded breaks with 3′ single stranded flaps;
the clkDNA oligonucleotide templates bind to the 3′ single stranded flaps;
the polymerase extends the 3′ single stranded flaps using the ACO complementary portion of the clkDNA as a template, thereby producing a modified nucleic acid of DNA comprising 3′ overhangs comprising an adaptor-complementary overhang (ACO) sequence, and
incubating the sample under conditions wherein the thermostable ligase ligates the dsDNA oligo to the modified nucleic acid of DNA, thereby producing product nucleic acid of DNA having ends comprised of defined adaptor sequences.

12. A method for generating a product nucleic acid of DNA having ends comprised of defined adaptor sequences, the method comprising:
(A) providing a reaction mixture prepared by the method of claim 7, comprising
(i) a DNA binding domain nuclease (DBDn), optionally Cas9;
(ii) one, two, or more pegRNAs that bind to the DBDn and comprise a 3′ extension including a primer binding site (PBS) a reverse transcriptase template (RTT) encoding the complement of the ACO, a scaffold and spacer comprising a sequence complementary to a selected target sequence; and
optionally (iii) an RNA-dependent polymerase, optionally wherein the polymerase is linked or fused to the DBDn in the reaction mixture;
wherein (i)-(ii) or (i)-(iii) are added to the mixture in any order, and incubating the reaction mixture under conditions for the gRNAs to bind to the DBDn;
(B) providing a sample comprising a substrate nucleic acid of DNA, preferably genomic DNA isolated from a cell; and
contacting the sample with:
the reaction mixture of (A),
a RNA-dependent DNA polymerase if the polymerase is absent from the reaction mixture,
a single stranded DNA oligonucleotide modified with a click-compatible moiety at the 5′ end (ssDNA click oligo) comprising a sequence complementary to the ACO sequence, and
a thermostable ligase,
incubating the sample under conditions wherein:
the nuclease induces a pair of double-stranded breaks with 3′ single stranded flaps;
the clkDNA oligonucleotide templates bind to the 3′ single stranded flaps;
the polymerase extends the 3′ single stranded flaps using the ACO complementary portion of the clkDNA as a template, thereby producing a modified nucleic acid of DNA comprising 3′ overhangs comprising an adaptor-complementary overhang (ACO) sequence, and
incubating the sample under conditions wherein the thermostable ligase is active, for the ssDNA click oligo to anneal to the 3′ overhangs and be ligated to the modified nucleic acid of DNA, thereby producing a further modified nucleic acid of DNA having 5′ ends comprising click-compatible moieties; and
contacting the further modified nucleic acid of DNA having 5′ ends comprising click-compatible moieties with dsDNA click oligos comprising defined adaptor sequences and a 3′ click compatible moiety that reacts with the click-compatible moieties on the further modified nucleic acid of DNA to attach the dsDNA click oligos to the ends of the further modified nucleic acid of DNA, thereby producing a product nucleic acid of DNA having ends comprised of defined adaptor sequences.

13. A method comprising:
preparing a reaction mixture comprising:
(i) a RNA binding domain nuclease (RBDn), optionally Cas13, optionally linked or fused to a clkDNA tethering domain;
(ii) at least one guide RNA (gRNA) that binds to the RBDn and comprises a spacer comprising a sequence complementary to a selected target sequence;
(iii) at least one clkDNA oligonucleotide template comprising a localization moiety that binds to the clkDNA tethering domain, a template encoding the complement of the adaptor-complementary overhang (ACO) sequence (cACO), and a flap binding region (FBR), and
optionally (iv) a DNA-dependent DNA polymerase, optionally wherein the polymerase is linked or fused to the RBDn in the reaction mixture;
wherein (i)-(iii) or (i)-(iv) are added to the mixture in any order, and incubating the reaction mixture under conditions for the gRNAs to bind to the RBDn and the clkDNA oligonucleotide templates to bind to the clkDNA tethering domain on the RBDn.

14. A method for generating a modified nucleic acid of RNA comprising a 3′ end comprising an adaptor-complementary overhang (ACO) sequence, the method comprising:
(A) providing a reaction mixture prepared by the method of claim 13, comprising:
(i) a RNA binding domain nuclease (RBDn), optionally Cas13, optionally linked or fused to a clkDNA tethering domain;
(ii) at least one guide RNA (gRNA) that binds to the RBDn and comprises a spacer comprising a sequence complementary to a selected target sequence;
(iii) at least one clkDNA oligonucleotide template comprising a localization moiety that binds to the clkDNA tethering domain, a template encoding the complement of the adaptor-complementary overhang (ACO) sequence, and a flap binding region (FBR), and
optionally (iv) a DNA-dependent DNA polymerase, optionally wherein the polymerase is linked or fused to the RBDn in the reaction mixture;
wherein (i)-(iii) or (i)-(iv) are added to the mixture in any order, and incubating the reaction mixture under conditions for the gRNAs to bind to the RBDn and the clkDNA oligonucleotide templates to bind to the clkDNA tethering domain on the RBDn;
(B) providing a sample comprising a substrate nucleic acid of RNA, preferably RNA isolated from a cell or sample from an animal; and
contacting the sample with the reaction mixture of (A) and a DNA-dependent DNA polymerase if the polymerase is absent from the reaction mixture, incubating the sample under conditions wherein:
the clkDNA oligonucleotide template binds to the RNA; and
the polymerase extends the 3′ end of the RNA molecule using the ACO complementary region of the clkDNA as a template, thereby producing a modified nucleic acid comprising a 3′ end comprising an adaptor-complementary overhang (ACO) sequence.

15. A method of generating a product nucleic acid of RNA having an end comprised of a defined adaptor sequence, the method comprising:
providing a sample comprising a modified nucleic acid of RNA comprising a 3′ end comprising an ACO sequence produced by the method of claim 14;
optionally treating the sample to inactivate any active enzymes, optionally by heating the sample to about 72° C.;
contacting the modified nucleic acid with a double stranded DNA oligonucleotide comprising defined adaptor sequences and a 3′ overhang comprising a sequence complementary to the ACO sequence (dsDNA cACO oligo) and a ligase, under conditions sufficient for the ligase to ligate the dsDNA oligo to the modified nucleic acid, thereby producing a product nucleic acid having an end comprised of a defined adaptor sequence.

16. A method of generating a product nucleic acid of RNA having ends comprised of defined adaptor sequences, the method comprising:
providing a modified nucleic acid of RNA comprising a 3′ end comprising an ACO sequence produced by the method of claim 14;
contacting the modified nucleic acid of RNA with a ligase and a single stranded DNA oligonucleotide modified with a click-compatible moiety at the 5′ end (ssDNA click oligo) comprising a sequence complementary to the ACO sequence under conditions sufficient for the ssDNA click oligo to anneal to the 3′ end and be ligated to the modified nucleic acid of RNA, thereby producing a further modified nucleic acid of RNA having a 5′ end comprising a click-compatible moiety; and
contacting the further modified nucleic acid RNA having a 5′ end comprising a click-compatible moiety with dsDNA click oligos comprising defined adaptor sequences and a 3′ click compatible moiety that reacts with the click-compatible moieties on the further modified nucleic acid RNA to attach the dsDNA click oligos to the ends of the further modified nucleic acid of RNA, thereby producing an product nucleic acid of RNA having ends comprised of defined adaptor sequences.

17. A method for generating a product nucleic acid of RNA having ends comprised of defined adaptor sequences, the method comprising:
(A) providing a reaction mixture prepared by the method of claim 13, comprising:
(i) a RNA binding domain nuclease (RBDn), optionally Cas13, optionally linked or fused to a clkDNA tethering domain;
(ii) at least one guide RNA (gRNA) that binds to the RBDn and comprises a spacer comprising a sequence complementary to a selected target sequence;
(iii) at least one clkDNA oligonucleotide template comprising a localization moiety that binds to the clkDNA tethering domain, a template encoding the complement of the adaptor-complementary overhang (ACO) sequence (cACO), and a flap binding region (FBR), and
optionally (iv) a DNA-dependent DNA polymerase, optionally wherein the polymerase is linked or fused to the RBDn in the reaction mixture;
wherein (i)-(iii) or (i)-(iv) are added to the mixture in any order, and incubating the reaction mixture under conditions for the gRNAs to bind to the RBDn and the clkDNA oligonucleotide templates to bind to the clkDNA tethering domain on the RBDn;
(B) providing a sample comprising a substrate nucleic acid of RNA; and
contacting the sample with:
the reaction mixture of (A),
a RNA-dependent DNA polymerase if the polymerase is absent from the reaction mixture,
a double stranded DNA oligonucleotide comprising defined adaptor sequences and a 3′ overhang comprising a sequence complementary to the ACO sequence (dsDNA cACO oligo), and
a ligase, optionally a thermostable ligase,
incubating the sample under conditions wherein:
the clkDNA oligonucleotide templates bind to the RNA;
the polymerase extends the 3′ end using the ACO complementary portion of the clkDNA as a template, thereby producing a modified nucleic acid comprising a ds 3′ end comprising an adaptor-complementary overhang (ACO) sequence, and incubating the sample under conditions wherein the thermostable ligase ligates the dsDNA oligo to the modified nucleic acid, thereby producing an product nucleic acid having ends comprised of defined adaptor sequences.

18. A method for generating a product nucleic acid, optionally RNA/DNA, having ends comprised of defined adaptor sequences, the method comprising:
(A) providing a reaction mixture prepared by the method of claim 13, comprising:
(i) a RNA binding domain nuclease (RBDn), optionally Cas13, optionally linked or fused to a clkDNA tethering domain;
(ii) at least one guide RNA (gRNA) that binds to the RBDn and comprises a spacer comprising a sequence complementary to a selected target sequence;
(iii) at least one clkDNA oligonucleotide template comprising a localization moiety that binds to the clkDNA tethering domain, a template encoding the complement of the adaptor-complementary overhang (ACO) sequence (cACO), and a flap binding region (FBR), and
optionally (iv) a DNA-dependent DNA polymerase, optionally wherein the polymerase is linked or fused to the RBDn in the reaction mixture;
wherein (i)-(iii) or (i)-(iv) are added to the mixture in any order, and incubating the reaction mixture under conditions for the gRNAs to bind to the RBDn and the clkDNA oligonucleotide templates to bind to the clkDNA tethering domain on the RBDn;
(B) providing a sample comprising a substrate nucleic acid of RNA; and
contacting the sample with:
the reaction mixture of (A),
a RNA-dependent DNA polymerase if the polymerase is absent from the reaction mixture,
a single stranded DNA oligonucleotide modified with a click-compatible moiety at the 5′ end (ssDNA click oligo) comprising a sequence complementary to the ACO sequence, and
a thermostable ligase,
incubating the sample under conditions wherein:
the clkDNA oligonucleotide templates bind to the RNA;
the polymerase extends the 3′ end using the ACO complementary portion of the clkDNA as a template, thereby producing a modified nucleic acid comprising a 3′ end comprising an adaptor-complementary overhang (ACO) sequence, and incubating the sample under conditions wherein the thermostable ligase is active, for the ssDNA click oligo to anneal to the 3′ end and be ligated to the modified nucleic acid, thereby producing a further modified nucleic acid comprising an RNA with a region of dsDNA having a 5′ end comprising a click-compatible moiety; and
contacting the region of dsDNA having a 5′ end comprising a click-compatible moiety with dsDNA click oligos comprising defined adaptor sequences and a 3′ click compatible moiety that reacts with the click-compatible moieties on the further modified nucleic acid to attach the dsDNA click oligos to the ends of the further modified nucleic acid, thereby producing a product nucleic acid having ends comprised of defined adaptor sequences.

19. A method comprising:
preparing a reaction mixture comprising:
(i) a RNA binding domain nuclease (RBDn), optionally Cas13;
(ii) at least one pegRNA that binds to the RBDn and comprise a 3′ extension including a primer binding site (PBS) a reverse transcriptase template (RTT) encoding the complement of an ACO, a scaffold and spacer comprising a sequence complementary to a selected target sequence; and
optionally (iii) an RNA-dependent polymerase, optionally wherein the polymerase is linked or fused to the RBDn in the reaction mixture;
wherein (i)-(ii) or (i)-(iii) are added to the mixture in any order, and incubating the reaction mixture under conditions for the gRNAs to bind to the RBDn.

20. A method for generating a modified nucleic acid comprising a 3′ end comprising an adaptor-complementary overhang (ACO) sequence, the method comprising:
(A) providing a reaction mixture prepared according to claim 19, comprising (i) a RNA binding domain nuclease (RBDn), optionally Cas13;
(ii) at least one pegRNA that binds to the RBDn and comprise a 3′ extension including a primer binding site (PBS) a reverse transcriptase template (RTT) encoding the complement of an ACO, a scaffold and spacer comprising a sequence complementary to a selected target sequence; and
optionally (iii) an RNA-dependent polymerase, optionally wherein the polymerase is linked or fused to the RBDn in the reaction mixture;
wherein (i)-(ii) or (i)-(iii) are added to the mixture in any order, and incubating the reaction mixture under conditions for the gRNAs to bind to the RBDn;
(B) providing a sample comprising a substrate nucleic acid of RNA; and
contacting the sample with the reaction mixture of (A) and an RNA-dependent DNA polymerase if the polymerase is absent from the reaction mixture, incubating the sample under conditions wherein:
the clkDNA oligonucleotide templates bind to the RNA; and
the polymerase extends the 3′ end using the ACO complementary portion of the clkDNA as a template, thereby producing a modified nucleic acid comprising a 3′ end comprising an adaptor-complementary overhang (ACO) sequence.

21. A method of generating a product nucleic acid having ends comprised of defined adaptor sequences, the method comprising:
providing a sample comprising a modified nucleic acid comprising a 3′ end comprising ACO sequence produced by the method of claim 20;
optionally treating the sample to inactivate any active enzymes, optionally by heating the sample to about 72° C.;
contacting the modified nucleic acid with a double stranded DNA oligonucleotide comprising defined adaptor sequences and a 3′ overhang comprising a sequence complementary to the ACO sequence (dsDNA cACO oligo) and a ligase, under conditions sufficient for the ligase to ligate the dsDNA oligo to the modified nucleic acid, thereby producing a product nucleic acid having ends comprised of defined adaptor sequences.

22. A method of generating a product nucleic acid having ends comprised of defined adaptor sequences, the method comprising:
providing a modified nucleic acid comprising 3′ overhangs at comprising ACO sequence produced by the method of claim 20;
contacting the modified nucleic acid with a ligase and a single stranded DNA oligonucleotide modified with a click-compatible moiety at the 5′ end (ssDNA click oligo) comprising a sequence complementary to the ACO sequence under conditions sufficient for the ssDNA click oligo to anneal to the 3′ overhangs and be ligated to the modified nucleic acid, thereby producing a further modified nucleic acid having 5′ ends comprising click-compatible moieties; and
contacting the further modified nucleic acid having 5′ ends comprising click-compatible moieties with dsDNA click oligos comprising defined adaptor sequences and a 3′ click compatible moiety that reacts with the click-compatible moieties on the further modified nucleic acid to attach the dsDNA click oligos to the ends of the further modified nucleic acid, thereby producing a product nucleic acid having ends comprised of defined adaptor sequences.

23. A method for generating a product nucleic acid having ends comprised of defined adaptor sequences, the method comprising:
(A) providing a reaction mixture prepared by the method of claim 19, comprising
(i) a RNA binding domain nuclease (RBDn), optionally Cas13;
(ii) one, two, or more pegRNAs that bind to the RBDn and comprise a 3′ extension including a primer binding site (PBS) a reverse transcriptase template (RTT) encoding the complement of an ACO, a scaffold and spacer comprising a sequence complementary to a selected target sequence; and
optionally (iii) an RNA-dependent polymerase, optionally wherein the polymerase is linked or fused to the RBDn in the reaction mixture;
wherein (i)-(ii) or (i)-(iii) are added to the mixture in any order, and incubating the reaction mixture under conditions for the gRNAs to bind to the RBDn;
(B) providing a sample comprising a substrate nucleic acid of RNA; and
contacting the sample with:
the reaction mixture of (A),
a RNA-dependent DNA polymerase if the polymerase is absent from the reaction mixture,
a double stranded DNA oligonucleotide comprising defined adaptor sequences and a 3′ overhang comprising a sequence complementary to the ACO sequence (dsDNA cACO oligo), and
a ligase, optionally a thermostable ligase,
incubating the sample under conditions wherein:
the clkDNA oligonucleotide templates bind to the RNA;
the polymerase extends the 3′ end using the ACO complementary portion of the clkDNA as a template, thereby producing a modified nucleic acid comprising a 3′ end comprising an adaptor-complementary overhang (ACO) sequence, and incubating the sample under conditions wherein the thermostable ligase ligates the dsDNA oligo to the modified nucleic acid, thereby producing a product nucleic acid having an end comprised of a defined adaptor sequence.

24. A method for generating a product nucleic acid having ends comprised of defined adaptor sequences, the method comprising:
(A) providing a reaction mixture prepared by the method of claim 19, comprising
(i) a RNA binding domain nuclease (RBDn), optionally Cas13;
(ii) one, two, or more pegRNAs that bind to the RBDn and comprise a 3′ extension including a primer binding site (PBS) a reverse transcriptase template (RTT) encoding the complement of an ACO, a scaffold and spacer comprising a sequence complementary to a selected target sequence; and
optionally (iii) an RNA-dependent polymerase, optionally wherein the polymerase is linked or fused to the RBDn in the reaction mixture;
wherein (i)-(ii) or (i)-(iii) are added to the mixture in any order, and incubating the reaction mixture under conditions for the gRNAs to bind to the RBDn;
(B) providing a sample comprising a substrate nucleic acid of RNA; and
contacting the sample with:
the reaction mixture of (A),
a RNA-dependent DNA polymerase if the polymerase is absent from the reaction mixture,
a single stranded DNA click oligonucleotide modified with a click-compatible moiety at the 5′ end (ssDNA click oligo) comprising a sequence complementary to the ACO sequence, and
a thermostable ligase,
incubating the sample under conditions wherein:
the clkDNA oligonucleotide templates bind to the RNA;
the polymerase extends the 3′ single stranded flaps using the ACO complementary portion of the clkDNA as a template, thereby producing a modified nucleic acid comprising a 3′ end comprising an adaptor-complementary overhang (ACO) sequence, and
incubating the sample under conditions wherein the thermostable ligase is active, for the ssDNA click oligo to anneal to the 3′ end and be ligated to the modified nucleic acid, thereby producing a further modified nucleic acid having 5′ ends comprising click-compatible moieties; and
contacting the further modified nucleic acid having 5′ ends comprising click-compatible moieties with dsDNA oligos comprising defined adaptor sequences and a 3′ click compatible moiety that reacts with the click-compatible moieties on the further modified nucleic acid (dsDNA click oligo) to attach the dsDNA click oligos to the ends of the further modified nucleic acid, thereby producing a product nucleic acid having ends comprised of defined adaptor sequences.

25. A method for labeling DNA or RNA as described herein.

26. A method comprising:
cleaving a nucleic acid using a sequence-specific nuclease (optionally via a nuclease-active PCE construct comprising a CRISPR-Cas nuclease directed by a gRNA);
writing adaptor-complementary overhang (ACO) sequences onto a 3′ end of a cleaved nucleic acid (optionally via a DNA-dependent DNA polymerase of the PCE using the clkDNA as a template); and
ligating an oligonucleotide, optionally a sequencing adaptor, onto the 3′ ACO.

27. The method of claim 26, wherein the nucleic acid is DNA, and the nuclease is a Class II type II CRISPR, optionally CRISPR Cas9.

28. The method of claim 26, wherein the nucleic acid is RNA, and the nuclease is a class II type VI CRISPR, optionally CRISPR Cas13.

29. The method of claims 26-28, further comprising sequencing the nucleic acid using the sequencing adaptor.

30. Any of the previous claims, wherein the DBDn is a Cas-family enzyme (optionally Cas9 or Cas12), a TnpB-family enzyme, or a IscB-family enzyme.


SEQUENCE LISTING (96 sequences)

SEQ ID NO: 1 - protein, 28 aa, synthetic construct
EIQALEEENAQLEQENAALEEEIAQLEY

SEQ ID NO: 2 - protein, 28 aa, synthetic construct
KIAQLKEKNAALKEKNQQLKEKIQALKY

SEQ ID NO: 3 - protein, 28 aa, synthetic construct
EIQQLEEEIAQLEQKNAALKEKNQALKY

SEQ ID NO: 4 - protein, 28 aa, synthetic construct
KIAQLKQKIQALKQENQQLEEENAALEY

SEQ ID NO: 5 - protein, 28 aa, synthetic construct
EIQQLEEEISQLEQKNSQLKEKNQQLKY

SEQ ID NO: 6 - protein, 28 aa, synthetic construct
KISQLKQKIQQLKQENQQLEEENSQLEY

SEQ ID NO: 7 - protein, 28 aa, synthetic construct
ENAALEEKIAQLKQKNAALKEEIQALEY

SEQ ID NO: 8 - protein, 28 aa, synthetic construct
KNAALKEEIQALEEENQALEEKIAQLKY

SEQ ID NO: 9 - protein, 28 aa, synthetic construct
EIQALEEKNAQLKQEIAALEEKNQALKY

SEQ ID NO: 10 - protein, 28 aa, synthetic construct
KIAQLKEENQQLEQKIQALKEENAALEY

SEQ ID NO: 11 - protein, 28 aa, synthetic construct
ENQALEQKNAQLKQEIAALEQEIAQLEY

SEQ ID NO: 12 - protein, 28 aa, synthetic construct
KNAQLKEENAALEEKIQQLKEKIQALKY

SEQ ID NO: 13 - protein, 28 aa, synthetic construct
ENQALEQEIAQLEQEIAALEQKNAQLKY

SEQ ID NO: 14 - protein, 28 aa, synthetic construct
KNAQLKEKIAALKEKIQQLKEENQALEY

SEQ ID NO: 15 - protein, 28 aa, synthetic construct
EIAALEAKIAALKAKNAALKAEIAALEA

SEQ ID NO: 16 - protein, 28 aa, synthetic construct
KIAALKAEIAALEAENAALEAKIAALKA

SEQ ID NO: 17 - protein, 28 aa, synthetic construct
ELAANEEELQQNEQKLAQIKQKLQAIKY

SEQ ID NO: 18 - protein, 22 aa, synthetic construct
LLPKNYHLENEVARLKKLVGER

SEQ ID NO: 19 - protein, 19 aa, synthetic construct
EELLSKNYHLENEVARLKK

SEQ ID NO: 20 - protein, 246 aa, synthetic construct
GPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSS

SEQ ID NO: 21 - protein, 117 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQR

SEQ ID NO: 22 - protein, 120 aa, synthetic construct
MAKSGNYSYKRWVFTINNPTFEDYVHVLEFCTLDNCKFAIVGEEKGANGTPHLQGFLNLRSNARAAALEESLGGRAWLSRARGSDEDNEEYCAKESTYLRVGEPVSKGRSSDLAEATSAV

SEQ ID NO: 23 - protein, 98 aa, synthetic construct
MARQVICWCFTLNNPLSPLSLHDSMKYLVYQTEQGEAGNIHFQGYIEMKKRTSLAGMKKLIPGAHFEKRRGTQGEARAYSMKEDTRLEGPWEYGEFVP

SEQ ID NO: 24 - protein, 130 aa, synthetic construct
MSEKKEIVKGRDWTFLVYPESAPENWRTILDETFMRWVESPLHDKDVNADGEIKKPHWHILLSSDGPITQTAVQKIIGPLNAPNAQKVGSAKGLVRYMVHLDNPEKYQYSLDEIVGHNGADVASYFELTA

SEQ ID NO: 25 - protein, 330 aa, synthetic construct
MMSIAQVRSAGSAGNYYTDKDNYYVLGSMGERWAGRGAEQLGLQGSVDKDVFTRLLEGRLPDGADLSRMQDGSNRHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQTIREAVGEDASLKSRDVAALDTRKSKQHVDPEIKMAEWMQTLKETGFDIRAYRDAADQRADLRTLTPGPASQDGPDVQQAVTQAIAGLSER

SEQ ID NO: 26 - protein, 117 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEFCSKEGNLLMECGAPRSQGQR

SEQ ID NO: 27 - protein, 131 aa, synthetic construct
MAKEKARYFTFLLYPESIPSDWELKLETLGVPMAISPLHDKDKSSIKGQKYKKAHYHVLYIAKNPVTADSVRKKIKLLLGEKSLAMVQVVLNVENMYLYLTHESKDAIAKKKHVYDKADIKLINNFDIDRY

SEQ ID NO: 28 - protein, 286 aa, synthetic construct
MARYVVCWMFTINNPTTLPVMRDEIKYMVYQVERGQEGTRHVQGYVEMKRRSSLKQMRVFFPGAHLEKRKGSQEEARSYCMKEDTRIEGPFEFGAFKLSCNDNLFDVIQDMRETHKRPLEYLYDCPNTFDRSKDTLYRVQAELNKTRAMNSWRTSFSAWTSEVENIMAQPCHRRIIRVYGPNGGEGKTTYAKHLMKTKNAFYSPGGKSLDICRLYNYEDIVIFDIPRCKEDYLNYGLLEEFKNGIIQSGKYEPVLKVVEYVEVIVMANFLPKEGIFSEDRIKLVSC

SEQ ID NO: 29 - protein, 351 aa, synthetic construct
MASSSAPRFRVYSKYLFLTYPQCTLEPQYALDSLRTLLNKYEPLYIAAVRELHEDGSPHLHVLVQNKLRASITNPNALNLRMDTSPFSIFHPNIQAAKDCNQVRDYITKEVDSDANTAEWGTFVAVSTPGRKDRDADMKQIIESSSSREEFLSMVCNRFPFEWSIRLKDFEYTARHLFPDPVATYTPEFPTESLICHETIESWKNEHLYSESPGRHKSIYICGPTRTGKTSWARSLGTHNYYNSLVDFTTYDVNAKYNIIDDIPFKFTPNWKCFVGAQRDFTVNPKYGKRKVIRGGIPCIILVNPDEDWLKDMTPEQSDYMYSNAVVHYMYEGESFINYSFASGEDVTASQ

SEQ ID NO: 30 - protein, 357 aa, synthetic construct
MPRLFKIYAKNYFLTYPNCSLSKEEALSQLKNLETPTNKKYIKVCRELHENGEPHLHVLIQFEGKYQCKNQRFFDLVSPNRSAHFHPNIQAAKSSTDVKTYVEKDGDFIDFGVFQIDGRSARGGQQSANDAYAEALNSGNKSEALNILKEKAPKDYILQFHNLSSNLDRIFSPPLEVYVSPFLSSSFNQVPDELEEWVAENVVSSAARPWRPNSIVIEGDSRTGKTMWARSLGPHNYLCGHLDLSPKVYSNDAWYNVIDDVDPHYLKHFKEFMGAQRDWQSNTKYGKPIQIKGGIPTIFLCNPGPTSSYREYLDEEKNISLKNWALKNATFVTLYEPLFASINQGPTQDSQEETNKA

SEQ ID NO: 31 - protein, 334 aa, synthetic construct
MPSANKNFRFQSKYVFLTYPKCSSQRDDLFEFLWEKLTPFLIFFLGVATELHQDGTTHYHALIQLDKKPWIRDPSFFDFEGNHPNIQPARNSKQVLEYISKDGDIKTRGDFRDHKVSPRKSDARWRTIIQTATSKEEYLDMIKEEFPHEWATKLQWLEYSANKLFPPQPEAYVSPFTESDLRCHEDLAQWRDTHLYQEPRRTGARVPSLYICGPTRTGKTTWARSLGRHNYWNGTIDFTTYDEHATYNVIDDIPFKFVPLWKQLIGCQFDFTVNPKYGKKKKIKGGVPSIILTNRDEDWIPAMSEHQKEYFTDNCEIHYMDDGETFFARESSSH

SEQ ID NO: 32 - protein, 343 aa, synthetic construct
MSHTSFRFRAKNVFLTYPRCPIGPEFLCDHLWNLVTPYDPLYVHVAQENHKDGGLHSHVLIQTRIEISTFDPTYFDYTGTSIPGAVVFHPNIQACRNVRDCLAYIRKNTINEVSKGAFKCSGAGRPKKQDSAPSRDAKMCQIMSSATSRSDYLSMVRGAFPFEWATKLAQFEYSASKLFPDAPTTNAIPSNIDLTCHENIMPWLRDDLYTERGQGRRPRSLYICGRTRTGKTTWARSLGPHNYFQHQVDYFAWSEEATYVVIDDIPFKFCPNWKAIVGSQHDYTVNPKYGKKRKLKGGIPAIILVNEDEDWLMSMTTSQIDWFLENCVVYHMTPGESFFNYAE

SEQ ID NO: 33 - protein, 349 aa, synthetic construct
MPRNPKSFRLAARNIFLTYPQCDIPKDEALQMLQTLSWSVVKPTYIRVAREEHSDGSPHLHCLIQLSGKSNIKDARLFDITHPRRSANFHPNIQAAKDTNAVKNYITKDGDYCESGQYKVSGGTKANKDDVYHNAVNAGCVEEALAIIKAGDPKTFIVSYHNVRANIERLFTKAPEPWAPPFQLSSFTNVPDEMRSWADDYFGRSAAARPERPISIIVEGDSRTGKTMWARALGPHNYLSGHLDFNSKVFSNNVEYNVIDDIAPHYLKLKHWKELIGAQRDWQSNCKYGRPVQIKGGIPSIVLCNPGEGSSYISFLNKEENASLRAWTIKNAKFITLEAPLYQSTAQDC

SEQ ID NO: 34 - protein, 352 aa, synthetic construct
MPSHPKRFQINAKNYFLTYPQCSLSKEESLSQLQALNTPINKKFIKICRELHEDGQPHLHVLIQFEGKYCCQNQRFFDLVSPTRSAHFHPNIQRAKSSSDVKTYIDKDGDTLVWGEFQVDGRSARGGCQTSNDAAAEALNASSKEEALQIIREKIPEKYLFQFHNLNSNLDRIFDKTPEPWLPPFHVSSFTNVPDEMRQWAENYFGKSSAARPERPISIIIEGDSRTGKTMWARSLGPHNYLSGHLDLNSRVYSNKVEYNVIDDVTPQYLKLKHWKELIGAQRDWQTNCKYGKPVQIKGGIPSIVLCNPGEGASYKVFLDKEENTPLKNWTFHNAKFVFLNSPLYQSSTQSS

SEQ ID NO: 35 - protein, 298 aa, synthetic construct
MLSHMVLTRQDIGRAASYYEDGADDYYAKDGDASEWQGKGAEELGLSGEVDSKRFRELLAGNIGEGHRIMRSATRQDSKERIGLDLTFSAPKSVSLQALVAGDAEIIKAHDRAVARTLEQAEARAQARQKIQGKTRIETTGNLVIGKFRHETSRERDPQLHTHAVILNMTKRSDGQWRALKNDEIVKRTRYLGAVYNAELAHELQKLGYQLRYGKDGNFDLAHIDRQQIEGFSKRTEQIAEWYAARGLDPNSVSLEQKQAAKVLSRAKKTSVDREALRAEWQATAKELGIDFSRREWS

SEQ ID NO: 36 - protein, 220 aa, synthetic construct
MAMYHFQNKFVSKANGQSATAKSAYNSASRIKDFKENEFKDYSNKQCDYSEILLPNNADDKFKDREYLWNKVHDVENRKNSQVAREIIIGLPNEFDPNSNIELAKEFAESLSNEGMIVDLNIHKINEENPHAHLLCTLRGLDKNNEFEPKRKGNDYIRDWNTKEKHNEWRKRWENVQNKHLEKNGFSVRVSADSYKNQNIDLEPTKKEGWKARKFEDETG

SEQ ID NO: 37 - protein, 186 aa, synthetic construct
MAIYHLTAKTGSRSGGQSARAKADYIQREGKYARDMDEVLHAESGHMPEFVERPADYWDAADLYERANGRLFKEVEFALPVELTLDQQKALASEFAQHLTGAERLPYTLAIHAGGGENPHCHLMISERINDGIERPAAQWFKRYNGKTPEKGGAQKTEALKPKAWLEQTREAWADHANRALERAGH

SEQ ID NO: 38 - protein, 271 aa, synthetic construct
MHLFTDVVSVDIMTTMQRTHTNAELIRWFKIFRDLDIHKWVIGLEEGKGGYGHWQVRCNVRVEVVNDWTAYLRAVFGWLGPISIWTEECSDKYTYETKEGKYWASWDTMGARQQRFGKMRWNQEGAVQALQRTNDREIVVWYDEQGNMGKSWLCGHLFETGQAYYIPPYMTSIQSMIQTVASLVLQDRDSGYPPRPLIVIDIPRSWKWSTELYTAIEAIKDGLIMDPRYGARPVNIHGIKVIVLTNTKPKLDKLSEDRWVLYDPMDYLMML

SEQ ID NO: 39 - protein, 271 aa, synthetic construct
MHLFTEGISVDIMTTMQRTHTNAELIRWFKIFRDLDIHKWVIGLEEGKGEYGHWQVRCNVRVEVVNDWTAYLRAVFGWLGPISIWTEECSDKYTYETKEGKYWASWDTMGARQQRFGKMRWNQEGAVQALQRTNDREIVVWYDEGGNMGKSWLCGHLFETGQAYYIPPYMTSIQSMIQTVASLVLQDRETGYPPRPLIVIDIPRSWKWSTELYTAIEAIKGGLIMDPRYGARPVNIHGTKVIVLTNTKPKLDKLSEDRWVLYDPMDYLMML

SEQ ID NO: 40 - protein, 163 aa, synthetic construct
MGARQQRFGKMRWNQEGALQALQRTNDREIVVWYDQDGNMGKSWLCGHLYETGQAYYIPPYMTSIQSMIQTVASLVLQDRESGYPPRPLIVIDIPRSRKWSTELYTAIEAIKDGLIMDPRYGARPVNIHGTKVIVLTNTKPKLDKLSEDRWVLYDPLLYMMGH

SEQ ID NO: 41 - DNA, 17 bp, synthetic construct
  source 1..17: mol_type = other DNA
ctgtaagtattaccagc

SEQ ID NO: 42 - DNA, 17 bp, synthetic construct
  source 1..17: mol_type = other DNA
gttatattattaccggc

SEQ ID NO: 43 - DNA, 17 bp, synthetic construct
  source 1..17: mol_type = other DNA
ggcttagtattaccccc

SEQ ID NO: 44 - DNA, 23 bp, synthetic construct
  source 1..23: mol_type = other DNA
tgcttccgtactacgacccccca

SEQ ID NO: 45 - DNA, 20 bp, synthetic construct
  source 1..20: mol_type = other DNA
tttgcgtggggtgtggtgct

SEQ ID NO: 46 - DNA, 17 bp, synthetic construct
  source 1..17: mol_type = other DNA
ggcttattattaccccc

SEQ ID NO: 47 - DNA, 17 bp, synthetic construct
  source 1..17: mol_type = other DNA
gttataatattaccccc

SEQ ID NO: 48 - DNA, 17 bp, synthetic construct
  source 1..17: mol_type = other DNA
cgtataatattaccgga

SEQ ID NO: 49 - DNA, 17 bp, synthetic construct
  source 1..17: mol_type = other DNA
aaattaatattaccggc

SEQ ID NO: 50 - DNA, 17 bp, synthetic construct
  source 1..17: mol_type = other DNA
ataataatattacgcgc

SEQ ID NO: 51 - DNA, 17 bp, synthetic construct
  source 1..17: mol_type = other DNA
gcaataatattaccgga

SEQ ID NO: 52 - DNA, 17 bp, synthetic construct
  source 1..17: mol_type = other DNA
cgtttaatattaccgga

SEQ ID NO: 53 - DNA, 27 bp, synthetic construct
  source 1..27: mol_type = other DNA
gcgcaccgaaaggtgcgtattgtctat

SEQ ID NO: 54 - DNA, 45 bp, synthetic construct
  source 1..45: mol_type = other DNA
acgcgaacggaacgttcgcataagtgcgcccttacgggatttaac

SEQ ID NO: 55 - DNA, 38 bp, synthetic construct
  source 1..38: mol_type = other DNA
ccagtttctcgaagagaaaccggtaagtgcaccctccc

SEQ ID NO: 56 - DNA, 14 bp, synthetic construct
  source 1..14: mol_type = other DNA
aatagttacgaaaa

SEQ ID NO: 57 - protein, 182 aa, synthetic construct
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 58 - protein, 182 aa, synthetic construct
MDKDCEMKRTTLDSPLGKLELSGCEQGLHRIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLG

SEQ ID NO: 59 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 60 - protein, 120 aa, synthetic construct
MAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAAS

SEQ ID NO: 61 - protein, 129 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTE

SEQ ID NO: 62 - protein, 495 aa, synthetic construct
MAVVYVISKSGKPLMPTTRCGHVRILLKEGKARVVERKPFTIQLTYESAEETQPLVLGIDPGRTNIGMSVVTESGESVFNAQIETRNKDVPKLMKDRKQYRMAHRRLKRRCKRRRRAKAAGTAFEEGEKQRLLPGCFKPITCKSIRNKEARFNNRKRPVGWLTPTANHLLVTHLNVVKKVQKILPVAKVVLELNRFSFMAMNNPKVQRWQYQRGPLYGKGSVEEAVSMQQDGHCLFCKHGIDHYHHVVPRRKNGSETLENRVGLCEEHHRLVHTDKEWEANLASKKSGMNKKYHALSVLNQIIPYLADQLADMFPGNFCVTSGQDTYLFREEHGIPKDHYLDAYCIACSALTDAKKVSSPKGRPYMVHQFRRHDRQACHKANLNRSYYMGGKLVATNRHKAMDQKTDSLEEYRAAHSAADVSKLTVKHPSAQYKDMSRIMPGSILVSGEGKLFTLSRSEGRNKGQVNYFVSTEGIKYWARKCQYLRNNGGLQIYV

SEQ ID NO: 63 - protein, 427 aa, Eubacterium rectale
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 64 - protein, 597 aa, synthetic construct
MKSRKRRNRVSFLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHA

SEQ ID NO: 65 - protein, 420 aa, Geobacillus stearothermophilus
MALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 66 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 67 - protein, 1630 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSGTRLPKKKRKVGGGSHHHHHH

SEQ ID NO: 68 - protein, 1508 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 69 - protein, 605 aa, synthetic construct
VISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 70 - protein, 1368 aa, Streptococcus pyogenes [sequence omitted]

SEQ ID NO: 71 - protein, 691 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFE

SEQ ID NO: 72 - protein, 1261 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSMWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 73 - protein, 2147 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 74 - protein, 24 aa, synthetic construct
SGGSSGSETPGTSESATPESSGGS

SEQ ID NO: 75 - RNA, 20 nt, synthetic construct
  source 1..20: mol_type = other RNA
tagtataaacgaacaggatc

SEQ ID NO: 76 - RNA, 20 nt, synthetic construct
  source 1..20: mol_type = other RNA
ggcccagactgagcacgtga

SEQ ID NO: 77 - RNA, 34 nt, synthetic construct
  source 1..34: mol_type = other RNA
agacgtgtgctcttccgatctcgtgctcagtctg

SEQ ID NO: 78 - DNA, 45 bp, synthetic construct
  source 1..45: mol_type = other DNA
tgagtggccacactccggagtaactagtcattgccgaaggtctag

SEQ ID NO: 79 - DNA, 65 bp, synthetic construct
  source 1..65: mol_type = other DNA
ctagaccttcggcaatgactagttactccggagtgtggccactcagcaacacctcggctagcatc

SEQ ID NO: 80 - DNA, 19 bp, synthetic construct
  source 1..19: mol_type = other DNA
atgggacttcagcatggcg

SEQ ID NO: 81 - DNA, 20 bp, synthetic construct
  source 1..20: mol_type = other DNA
atcgctgagataggtgcctc

SEQ ID NO: 82 - DNA, 21 bp, synthetic construct
  source 1..21: mol_type = other DNA
tagttactccggagtgtggcc

SEQ ID NO: 83 - DNA, 20 bp, synthetic construct
  source 1..20: mol_type = other DNA
tccccgaaaagtgccacctg

SEQ ID NO: 84 - DNA, 22 bp, synthetic construct
  source 1..22: mol_type = other DNA
agggaaacgcccatgcaattag

SEQ ID NO: 85 - DNA, 59 bp, synthetic construct
  source 1..59: mol_type = other DNA
acactctttccctacacgacgctcttccgatctcctagaaaggcatggatgagagaagc

SEQ ID NO: 86 - DNA, 50 bp, synthetic construct
  source 1..50: mol_type = other DNA
gactggagttcagacgtgtgctcttccgatctcccccccccccccccccc

SEQ ID NO: 87 - DNA, 22 bp, synthetic construct
  source 1..22: mol_type = other DNA
tctgacctgaaggctctgcgcg

SEQ ID NO: 88 - DNA, 33 bp, synthetic construct
  source 1..33: mol_type = other DNA
acgttcgcgtttgccgtgcgtgtaatgtagtac

SEQ ID NO: 89 - DNA, 29 bp, synthetic construct
  source 1..29: mol_type = other DNA
caagcctacaaatgcatgcttacttgcag

SEQ ID NO: 90 - DNA, 55 bp, synthetic construct
  source 1..55: mol_type = other DNA
  modified_base 53..55: mod_base = OTHER; note = Phosphorothioate linkage
ctgtaagtattaccagcgcaacacctcggctagcatccctgttcgtttatactag

SEQ ID NO: 91 - DNA, 54 bp, synthetic construct
  source 1..54: mol_type = other DNA
  modified_base 52..54: mod_base = OTHER; note = Phosphorothioate linkage
ctgtaagtattaccagcgcaacacctcggctagcatccgtgctcagtctgggcc

SEQ ID NO: 92 - DNA, 1168 bp, synthetic construct
  source 1..1168: mol_type = other DNA
catgggacttcagcatggcggtgtttgcagattacgcgagcgggttctgacctgaaggctctgcgcggatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcggacgttcgcgtttgccgtgcgtgtaatgtagtactagtataaacgaacaggatcaggccaaattcctgaata

SEQ ID NO: 93 - protein, 6 aa, synthetic construct
HHHHHH

SEQ ID NO: 94 - DNA, 63 bp, synthetic construct
  source 1..63: mol_type = other DNA
ctagtataaacgaacagggatgctagccgaggtgttgctgagtggccacactccggagtaact

SEQ ID NO: 95 - DNA, 75 bp, synthetic construct
  source 1..75: mol_type = other DNA
atcctctgggagcccagactgagcacggatgctagccgaggtgttgctgagtggccacactactcgggagtaact

SEQ ID NO: 96 - DNA, 65 bp, synthetic construct
  source 1..65: mol_type = other DNA
gggtcaatccttggggcccagactgagcacgagatcggaagagcacacgtctcnacaaatgcatg